(19) United States
(12) Patent Application Publication
(10) Pub. No.: US 2026/0232425 A1
(43) Pub. Date: Aug. 13, 2026

(54) Transcatheter Devices And Methods For Pulmonary Flow Reduction In Patients With Congenital Heart Disease

(71) Applicant: Renata Medical, Inc., Newport Beach, CA (US)

(72) Inventors: Eason Abbott, Santa Monica, CA (US); Dustin Armer, Costa Mesa, CA (US); Corey Marshall, Newport Beach, CA (US); Jason Provol, San Diego, CA (US); Evan Zahn, Los Angeles, CA (US); Tim Walters, Lake Forest, CA (US)

(21) Appl. No.: 19/450,462

(22) Filed: Jan. 15, 2026

Related U.S. Application Data
(60) Provisional application No. 63/745,801, filed on Jan. 16, 2025.

(51) Int. Cl. A61F 2/06; A61B 17/12

(52) U.S. Cl. CPC A61F 2/06; A61B 17/12036; A61B 17/12109; A61B 17/12172; A61F 2002/068; A61F 2210/0014; A61F 2230/0078; A61F 2250/0039

(57) ABSTRACT

A transcatheter pulmonary flow reduction device for treating patients with congenital cardiac conditions and methods for making and using same. The transcatheter pulmonary flow reduction device comprises an hourglass-shaped device frame with a proximal end region, an intermediate waist region and a distal end region that cooperate to define an internal channel.

The device frame is deployed within a pulmonary artery of a patient and expanded such that the proximal and distal end regions engage the pulmonary artery and the internal channel of the waist region has a first expanded cross-section for restricting blood flow. Later, the waist region of the deployed device frame is further expanded for increasing the internal channel of the waist region from the first expanded cross-section to a second expanded cross-section that is greater than the first cross-section. The transcatheter pulmonary flow reduction device thereby adjusts the restricted blood flow through the pulmonary artery.

### CROSS-REFERENCE TO RELATED APPLICATIONS

This application claims the benefit of, and priority to, U.S. Provisional Application Ser. No. 63/745,801, filed on Jan. 16, 2025, the disclosure of which is hereby incorporated herein by reference in its entirety and for all purposes.

### FIELD

The disclosed embodiments relate generally to the field of medical devices and more particularly, but not exclusively, to medical stents, reducers, and other transcatheter or surgical devices for reducing blood flow in pulmonary arteries of neonatal, infant, and other pediatric patients.

### BACKGROUND

Historically, diameters of pulmonary artery and branch pulmonary arteries have been surgically reduced to treat patients with congenital heart disease (CHD) by restricting pulmonary blood flow. Pulmonary artery flow reduction is a palliative measure intended to prevent pulmonary overcirculation and pulmonary hypertension, thereby protecting the pulmonary vasculature and preparing the patient for subsequent staged reconstructive surgeries. For many years, the definitive treatment for excessive pulmonary blood flow was pulmonary artery banding (PAB), an open-heart surgical technique in which a constrictive band is placed around the main or branch pulmonary artery to mechanically reduce vessel diameter and flow.

Although widely practiced, PAB remains an invasive procedure, requiring cardiopulmonary bypass in some cases and thoracotomy in all cases. Such open surgical procedures are associated with prolonged recovery time, risk of infection, and negative developmental effects, particularly in neonatal and other pediatric patients. Furthermore, PAB is non-adjustable after implantation, making it difficult to fine-tune the degree of flow restriction postoperatively. The initial calibration of the band is highly dependent on the surgeon's experience and intraoperative hemodynamics, which may not reflect the patient's evolving physiology over time. As a result, patients may experience either inadequate restriction (leading to pulmonary overcirculation) or excessive restriction (causing hypoxemia or ventricular dysfunction).

Pulmonary artery banding is often employed as a temporary palliative step in patients with congenital lesions such as large ventricular septal defects (VSD), complete atrioventricular canal defects, or single-ventricle physiology (e.g., hypoplastic left heart syndrome). In such cases, controlled pulmonary flow reduction allows the patient to grow and stabilize before undergoing definitive reparative or staged surgical procedures, such as the Norwood, Glenn, or Fontan operations. However, the inability to adjust or remove the surgical band noninvasively remains a significant limitation in clinical management.

With the advancement of transcatheter therapies, less invasive alternatives to traditional surgery have emerged, including balloon dilation, covered stents, and occlusion devices. These techniques have demonstrated the potential to reduce complications, shorten recovery times, and improve outcomes in pediatric CHD patients. However, to date, no commercially available transcatheter devices are specifically designed to perform adjustable pulmonary flow restriction in neonates or infants.

In view of the foregoing, an unmet need exists for minimally invasive, adjustable, and/or retrievable transcatheter pulmonary flow reduction systems (or devices) that overcome the aforementioned obstacles and deficiencies of currently-available devices and therapies for controlled reduction of pulmonary blood flow. Such transcatheter pulmonary flow reduction systems advantageously can achieve controlled pulmonary artery flow reduction without the risks and recovery associated with open-heart surgery. By permitting flow resistance to be fine-tuned or otherwise adjusted, for example, the transcatheter pulmonary flow reduction systems can allow for percutaneous adjustment or removal and/or can be compatible with small delivery systems suitable for use on neonatal, infant, toddlers, young children and other pediatric patients.

### SUMMARY

The present disclosure relates to transcatheter pulmonary flow reduction devices (or means) for treating patients with congenital cardiac conditions and methods for making and using the same. The transcatheter pulmonary flow reduction device can comprise an hourglass-shaped device frame with a proximal frame end region, an intermediate waist region and a distal frame end region that cooperate to define an internal channel. The device frame is deployed within a pulmonary artery of a patient and radially expanded such that the proximal and distal frame end regions engage the pulmonary artery and the internal channel of the waist region has a first expanded cross-section for restricting the blood flow. Later, the waist region can be further expanded for increasing the internal channel of the waist region from the first expanded cross-section to a second expanded cross-section that is greater than the first cross-section. The transcatheter pulmonary flow reduction device thereby can adjust the restricted blood flow through the pulmonary artery.

In accordance with a first aspect disclosed herein, there is set forth a transcatheter pulmonary flow reduction device for adjusting blood flow through a pulmonary artery of a patient, wherein the transcatheter pulmonary flow reduction device can comprise:
a flexible device frame having an annular waist region in axial alignment between an annular proximal frame end region and an annular distal frame end region and being in an implantation state for facilitating insertion into the pulmonary artery of the patient, the annular proximal frame end region, the annular waist region and the annular distal frame end region each defining an external periphery and cooperating to define an internal channel extending from the annular proximal frame end region to the annular distal frame end region,
wherein the device frame can be configured to be deployed within the pulmonary artery and radially expanded from the implantation state to a first stable expanded state with the external periphery of the annular proximal frame end region and the external periphery of the annular distal frame end region each being adapted to engage the pulmonary artery and with the internal channel of the annular waist region having an internal cross-section being adapted to radially expand to a predetermined first cross-section that is less than a cross-section of the pulmonary artery for restricting the blood flow through the pulmonary artery via the device frame, and/or
wherein the deployed device frame can be configured to be subsequently radially re-expanded from the first stable expanded state to a second stable expanded state with the internal cross-section of the annular waist region being further expanded to a predetermined second cross-section that is less than the cross-section of the pulmonary artery and that is greater than the predetermined first cross-section for adjusting the restricted blood flow through the pulmonary artery via the device frame. The device frame, in other words, can be configured to be radially expanded from the first stable expanded state to a second stable expanded state after being deployed within the pulmonary artery and radially expanded from the implantation state to the first stable expanded state.

In selected embodiments, the transcatheter pulmonary flow reduction device of the first aspect can be configured for adjusting blood flow in a pediatric patient. The transcatheter pulmonary flow reduction device, for example, can be configured for adjusting blood flow in neonatal, infant, toddlers, young children and other pediatric patients. Additionally and/or alternatively, the transcatheter pulmonary flow reduction device can be configured for adjusting blood flow in an adult patient.

In selected embodiments of the transcatheter pulmonary flow reduction device of the first aspect, the external periphery of the annular proximal frame end region and the external periphery of the annular distal frame end region each can be adapted to engage an internal surface (or vessel wall) of the pulmonary artery. The external periphery of the annular proximal frame end region and the external periphery of the annular distal frame end region optionally can be adapted to maintain the engagement with the pulmonary artery when the device frame is in the second stable expanded state. Additionally and/or alternatively, the external peripheries of the annular proximal frame end region, the annular waist region and the annular distal frame end region can define an hourglass shape in the first stable expanded state and optionally can maintain the hourglass shape in the second stable expanded state.

In selected embodiments, the transcatheter pulmonary flow reduction device of the first aspect can be configured for implantation and/or deployment within the pulmonary artery of the patient via a surgical procedure. Additionally and/or alternatively, the transcatheter pulmonary flow reduction device of the first aspect can be configured for implantation and/or deployment within the pulmonary artery of the patient via a delivery catheter system (or means). The device frame of the transcatheter pulmonary flow reduction device of the first aspect optionally can be configured for expansion from the implantation state to the first stable expanded state via the delivery catheter system. Additionally and/or alternatively, the device frame of the transcatheter pulmonary flow reduction device of the first aspect optionally can be configured for expansion from the implantation state to the first stable expanded state via a balloon catheter system (or means) or other expansion catheter system (or means). The expansion catheter system optionally can be integrated with, or separate from, the delivery catheter system.

In selected embodiments of the transcatheter pulmonary flow reduction device of the first aspect, the device frame can comprise a self-expanding device frame. The device frame, for example, can be formed from a shape-memory alloy. Additionally and/or alternatively, the device frame can be formed from stainless steel, a cobalt-chromium alloy or any combination thereof.

In selected embodiments of the transcatheter pulmonary flow reduction device of the first aspect, the device frame can be configured for re-expansion from the first stable expanded state to the second stable expanded state via an expansion catheter system (or means).

In selected embodiments of the transcatheter pulmonary flow reduction device of the first aspect, the device frame can be configured for re-expansion from the first stable expanded state to the second stable expanded state via a second medical procedure that is subsequent to a first medical procedure during which the device frame can be expanded from the implantation state to the first stable expanded state.

In selected embodiments of the transcatheter pulmonary flow reduction device of the first aspect, the annular proximal frame end region of the device frame can comprise a first annular arrangement of device frame struts and/or the annular distal frame end region of the device frame can comprise a second annular arrangement of device frame struts. The first and second annular arrangements of device frame struts, for example, can include at least one elongated frame strut that extends from the annular proximal frame end region to the annular distal frame end region of the device frame. The first and second annular arrangements of device frame struts optionally can comprise a plurality of elongated frame struts that extend from the annular proximal frame end region to the annular distal frame end region of the device frame. Additionally and/or alternatively, the first and second annular arrangements of device frame struts can comprise a plurality of meandering device frame struts that defines one or more circumferential rows of frame cells disposed around the external periphery of the device frame.

The frame cells, for example, can be defined between respective pairs of adjacent device frame struts. In selected embodiments, the circumferential rows of frame cells can include at least one circumferential row of growth frame cells. Additionally and/or alternatively, the at least one circumferential row of growth frame cells can be associated with the annular waist region of the device frame. Each of the growth frame cells associated with the annular waist region of the device frame optionally can have a first dimension when the device frame is in the first stable expanded state and a second dimension that is greater than the first dimension when the device frame is in the second stable expanded state.

In selected embodiments, the transcatheter pulmonary flow reduction device of the first aspect can define a predetermined pattern of frame cells. The device frame of the transcatheter pulmonary flow reduction device, for example, can define the predetermined pattern of frame cells. The predetermined pattern of frame cells optionally can include a plurality of circumferential rows of frame cells disposed around the external periphery of the device frame, each of the circumferential rows having a predetermined number of frame cells, wherein at least one of the circumferential rows of frame cells comprises a predetermined number of re-expandable growth frame cells.

The plurality of circumferential rows of frame cells, additionally and/or alternatively, can include at least one proximal circumferential row of frame cells being associated with the proximal frame end region of the device frame, at least one distal circumferential row of frame cells being associated with the distal frame end region of the device frame and at least one central circumferential row of frame cells being disposed between the at least one proximal circumferential row of frame cells and the at least one distal circumferential row of frame cells and being associated with the annular waist region of the device frame. The at least one central circumferential row of frame cells optionally can comprise a predetermined number of re-expandable growth frame cells. The predetermined pattern of frame cells, for example, can include three circumferential rows of frame cells disposed around the external periphery of the device frame with each of the circumferential rows having six frame cells, three circumferential rows of frame cells disposed around the external periphery of the device frame with each of the circumferential rows having eight frame cells or three circumferential rows of frame cells disposed around the external periphery of the device frame with each of the circumferential rows having ten frame cells.

In selected embodiments, the transcatheter pulmonary flow reduction device of the first aspect can further comprise a device retrieval system (or means) being coupled (or otherwise integrated) with the device frame and configured for facilitating at least one of re-expansion, recapture, repositioning, retrieval and removal of the device frame after deployment of the device frame after deployment. The device retrieval system, for example, can comprise one or more device retrieval members each having a proximal retrieval member end region being coupled with the annular proximal frame end region and a distal retrieval member end region extending proximally from the device frame. In selected embodiments, the device retrieval system optionally can comprise one or more device retrieval members each having a proximal retrieval member end region being coupled with the annular distal frame end region and a distal retrieval member end region extending distally from the device frame. Additionally and/or alternatively, the distal retrieval member end region of at least one device retrieval member can include a device engagement system (or means) for enhancing an engagement between the at least one device retrieval member and an implant retrieval system (or means) for retrieving the device frame after deployment. The device engagement system alternatively can comprise a first device engagement system (or means) with a square profile, a second device engagement system (or means) with a T-shaped profile and/or a third device engagement system (or means) with a round profile.

In selected embodiments of the first aspect, the distal retrieval member end region of the one or more device retrieval members extend proximally and radially inwardly toward a longitudinal axis of the internal channel defined by the device frame. The distal retrieval member end region of the one or more device retrieval members, for example, can converge. The converging distal retrieval member end region of the one or more device retrieval members optionally can form or otherwise be integrated with a capture member for engaging an implant retrieval system (or means) and/or can converge at a coupling device (or means) for engaging an implant retrieval system (or means). In selected embodiments, the coupling means can comprise a coupler housing system (or means) for defining one or more retention openings for enhancing an engagement with the implant retrieval system. The coupler housing means can enhance the engagement with the implant retrieval system, for example, by receiving a coupler paddle system (or means) of the implant retrieval system within the one or more retention openings defined by the coupler housing means.

In selected embodiments, the transcatheter pulmonary flow reduction device of the first aspect can further comprise a device retrieval system (or means) being integrated with the device frame and being configured for facilitating at least one of re-expansion, recapture, repositioning, retrieval and removal of the device frame after deployment. The device retrieval means, for example, can comprise one or more device retrieval members each having a proximal retrieval member end region being integrated with the annular proximal frame end region and a distal retrieval member end region extending proximally from the device frame. The distal retrieval member end region of at least one device retrieval member optionally can include a device engagement system (or means) for enhancing an engagement between the at least one device retrieval member and an implant retrieval system (or means) for retrieving the device frame after deployment. The device engagement system alternatively can comprise a first device engagement system (or means) with a square profile, a second device engagement system (or means) with a T-shaped profile and/or a third device engagement system (or means) with a round profile.

In selected embodiments of the first aspect, the distal retrieval member end region of the one or more device retrieval members extend proximally and radially inwardly toward a longitudinal axis of the internal channel defined by the device frame. The distal retrieval member end region of the one or more device retrieval members, for example, can converge. The converging distal retrieval member end region of the one or more device retrieval members optionally can form or otherwise be integrated with a capture member for engaging an implant retrieval system (or means) and/or can converge at a coupling device (or means) for engaging an implant retrieval system (or means). In selected embodiments, the coupling means can comprise a coupler housing system (or means) for defining one or more retention openings for enhancing an engagement with the implant retrieval system. The coupler housing means can enhance the engagement with the implant retrieval system, for example, by receiving a coupler paddle system (or means) of the implant retrieval system within the one or more retention openings defined by the coupler housing means.

In selected embodiments, the transcatheter pulmonary flow reduction device of the first aspect can further comprise a first annular cover member being disposed circumferentially about the external periphery of the device frame. The first annular cover member can provide a seal around the external periphery of the device frame. The first annular cover member optionally can extend from the annular proximal frame end region of the device frame to the annular waist region of the device frame. The first annular cover member for example, can provide a seal at one or more distal strut end regions of the device frame or may not provide a seal at one or more distal strut end regions of the device frame.

The first annular cover member, in selected embodiments, can include a central cover region being disposed around the first annular cover member at the annular waist region of the device frame. The central cover region of the first annular cover member can constrict the annular waist region of the device frame to define the predetermined first cross-section of the internal channel at the annular waist region of the device frame in the first stable expanded state. The central cover region of the first annular cover member optionally can be configured to expand during re-expansion of the device frame from the first stable expanded state to the second stable expanded state, the central cover region of the first annular cover member constricting the annular waist region of the device frame to define the predetermined second cross-section of the internal channel at the annular waist region of the device frame.

In selected embodiments, the transcatheter pulmonary flow reduction device of the first aspect can further comprise a first cover loop member that can be disposed around the first annular cover member at the annular waist region of the device frame. The first cover loop member, for example, can be configured to constrict the annular waist region of the device frame to define the predetermined first cross-section of the internal channel at the annular waist region in the first stable expanded state and/or to break open upon re-expansion of the device frame from the first stable expanded state to the second stable expanded state. Additionally and/or alternatively, the transcatheter pulmonary flow reduction device of the first aspect can further comprise a second cover loop member that can be disposed around the first annular cover member at the annular waist region of the device frame. The second cover loop member, for example, can be configured to constrict the annular waist region of the device frame to define the predetermined second cross-section of the internal channel at the annular waist region of the device frame in the second stable expanded state.

In selected embodiments, the transcatheter pulmonary flow reduction device of the first aspect can further comprise at least one expandable band member that can be disposed around the first annular cover member at the annular waist region of the device frame. The cover loop member, for example, can be configured to constrict the annular waist region of the device frame to define the predetermined first cross-section of the internal channel at the annular waist region of the device frame in the first stable expanded state. The cover loop member optionally can be configured to expand during re-expansion of the device frame from the first stable expanded state to the second stable expanded state, the expanded cover loop member constricting the annular waist region of the device frame to define the predetermined second cross-section of the internal channel at the annular waist region of the device frame.

In selected embodiments of the transcatheter pulmonary flow reduction device of the first aspect, the first annular cover member can extend from the annular proximal frame end region of the device frame to the annular distal frame end region of the device frame. The first annular cover member can define one or more optional cover member openings at the annular distal frame end region of the device frame.

In selected embodiments, the transcatheter pulmonary flow reduction device of the first aspect can further comprise a second annular cover member that can be disposed circumferentially about the external periphery of the annular distal frame end region of the device frame. The second annular cover member, for example, can provide a seal at one or more distal strut end regions of the annular distal frame end region of the device frame.

In accordance with a second aspect disclosed herein, there is set forth a method for manufacturing a transcatheter pulmonary flow reduction device for adjusting blood flow through a pulmonary artery of a patient. The method of the second aspect, for example, can comprise a method for manufacturing the transcatheter pulmonary flow reduction device of the first aspect. Additionally and/or alternatively, the method of the second aspect can comprise:
forming a flexible device frame having an annular waist region in axial alignment between an annular proximal frame end region and an annular distal frame end region and being in an implantation state for facilitating insertion into the pulmonary artery of the patient, the annular proximal frame end region, the annular waist region and the annular distal frame end region each defining an external periphery and cooperating to define an internal channel extending from the annular proximal frame end region to the annular distal frame end region,
wherein the device frame can be configured to be deployed within the pulmonary artery and radially expanded from the implantation state to a first stable expanded state with the external periphery of the annular proximal frame end region and the external periphery of the annular distal frame end region each being adapted to engage the pulmonary artery and with the internal channel of the annular waist region having an internal cross-section being adapted to radially expand to a predetermined first cross-section that is less than a cross-section of the pulmonary artery for restricting the blood flow through the pulmonary artery via the device frame, and/or
wherein the deployed device frame can be configured to be subsequently radially re-expanded from the first stable expanded state to a second stable expanded state with the internal cross-section of the annular waist region being further expanded to a predetermined second cross-section that is less than the cross-section of the pulmonary artery and that is greater than the predetermined first cross-section for adjusting the restricted blood flow through the pulmonary artery via the device frame.

In selected embodiments of the method of the second aspect, the forming of the flexible device frame can include forming the flexible device frame from a predetermined biocompatible frame material. The forming of the flexible device frame, for example, can comprise forming the flexible device frame from a biocompatible metal. Exemplary biocompatible metals can include a nickel-titanium alloy, a cobalt-chromium alloy and/or stainless steel, without limitation. Additionally and/or alternatively, the forming of the flexible device frame, for example, can comprise forming the flexible device frame from a plastic material.

The flexible device frame optionally can be formed from a plurality of layers of the predetermined biocompatible frame material. The forming of the flexible device frame, for example, can comprise forming the flexible device frame from a plurality of laminated layers of the predetermined biocompatible frame material and/or forming the flexible device frame from a plurality of bonded layers of the predetermined biocompatible frame material. The forming of the flexible device frame optionally can comprise forming the flexible device frame from a predetermined shape-memory frame material.

In selected embodiments of the method of the second aspect, the forming of the flexible device frame can comprise laser-cutting the flexible device frame from a tubular stock of device frame material. The forming of the flexible device frame, for example can comprise rolling sheet stock of device frame material into a tube of device frame material and/or laser-cutting the flexible device frame from the tube of device frame material. Additionally and/or alternatively, the method of the second aspect can include forming the annular proximal frame end region of the device frame as a first annular arrangement of device frame struts and/or forming the annular distal frame end region of the device frame as a second annular arrangement of device frame struts.

In selected embodiments, the method of the second aspect can include forming the device frame with a predetermined pattern of frame cells. The device frame, for example, can be formed with a plurality of circumferential rows of frame cells disposed around the external periphery of the device frame, each of the circumferential rows having a predetermined number of frame cells. The forming of the flexible device frame optionally can include forming the device frame with at least one of the circumferential rows of frame cells comprising a predetermined number of re-expandable growth frame cells. In selected embodiments, the forming of the device frame with the at least one of the circumferential rows of frame cells can include forming at least one proximal circumferential row of frame cells being associated with the proximal frame end region of the device frame, at least one distal circumferential row of frame cells being associated with the distal frame end region of the device frame and/or at least one central circumferential row of frame cells being disposed between the at least one proximal circumferential row of frame cells and the at least one distal circumferential row of frame cells and being associated with the annular waist region of the device frame. The forming of the flexible device frame, for example, can include forming the at least one central circumferential row of frame cells can comprise a predetermined number of re-expandable growth frame cells.

In selected embodiments of the method of the second aspect, the forming of the flexible device frame can include forming a device retrieval system (or means) at the proximal frame end region of the device frame, the device retrieval system being configured for facilitating at least one of re-expansion, recapture, repositioning, retrieval and removal of the device frame after deployment.

In selected embodiments, the method of the second aspect can further comprise at least one of electropolishing the formed device frame, shape-setting the formed device frame, performing surface passivation on the formed device frame and/or disposing at least one radiopaque coating on the formed device frame.

In selected embodiments, the method of the second aspect can further comprise disposing an annular cover member circumferentially about the external periphery of the formed device frame. The disposing of the annular cover member, for example, can include, but is not limited to, suturing the annular cover member to the external periphery of the formed device frame, laser bonding the annular cover member to the external periphery of the formed device frame, heat bonding the annular cover member to the external periphery of the formed device frame, adhesive bonding the annular cover member to the external periphery of the formed device frame, welding the annular cover member to the external periphery of the formed device frame, friction-fitting the annular cover member to the external periphery of the formed device frame, and/or encapsulation processing the annular cover member on the external periphery of the formed device frame, without limitation. Additionally and/or alternatively, the disposing of the annular cover member can include disposing a single-layer annular cover member circumferentially about the external periphery of the formed device frame and/or disposing a multiple-layer annular cover member circumferentially about the external periphery of the formed device frame, without limitation.

In accordance with a third aspect disclosed herein, there is set forth a catheter system (or means) for deploying a transcatheter pulmonary flow reduction device for adjusting blood flow through a pulmonary artery of a patient. The catheter system of the third aspect, for example, can comprise a catheter system (or means) for deploying the transcatheter pulmonary flow reduction device of the first aspect. Additionally and/or alternatively, the catheter system of the third aspect can comprise a catheter system for delivering a transcatheter pulmonary flow reduction device defining an internal channel with a controllable internal cross-section for adjusting blood flow through a pulmonary artery of a patient and can comprise:
a flexible delivery shaft member having a distal shaft end region for engaging the transcatheter pulmonary flow reduction device,
wherein the delivery shaft member can be configured to deliver the transcatheter pulmonary flow reduction device to the pulmonary artery of the patient for deployment in a first stable expanded state in which the internal cross-section can comprise a predetermined first cross-section that is less than a cross-section of the pulmonary artery for restricting the blood flow through the pulmonary artery via the transcatheter pulmonary flow reduction device, and/or
wherein the deployed transcatheter pulmonary flow reduction device can be configured to subsequently transition from the first stable expanded state to a second stable expanded state in which the internal cross-section can comprise a predetermined second cross-section that is less than the cross-section of the pulmonary artery and that is greater than the predetermined first cross-section for adjusting the restricted blood flow through the pulmonary artery via the transcatheter pulmonary flow reduction device.

In selected embodiments of the catheter system of the third aspect, the transcatheter pulmonary flow reduction device can comprise a flexible device frame having an annular waist region in axial alignment between an annular proximal frame end region and an annular distal frame end region and being in an implantation state for facilitating insertion into the pulmonary artery of the patient, the annular proximal frame end region, the annular waist region and the annular distal frame end region each defining an external periphery and cooperating to define the internal channel extending from the annular proximal frame end region to the annular distal frame end region. The distal shaft end region of the delivery shaft member, for example, can be configured to engage the annular proximal frame end region of the device frame. The device frame optionally can be configured to be deployed within the pulmonary artery and radially expanded from the implantation state to the first stable expanded state with the external periphery of the annular proximal frame end region and the external periphery of the annular distal frame end region each being adapted to engage the pulmonary artery and with the internal channel of the annular waist region being adapted to radially expand to the predetermined first cross-section. Additionally and/or alternatively, the device frame of the deployed transcatheter pulmonary flow reduction device can be configured to be subsequently radially re-expanded from the first stable expanded state to the second stable expanded state with the internal cross-section of the annular waist region being further expanded to the predetermined second cross-section.

In selected embodiments of the catheter system of the third aspect, the distal shaft end region of the delivery shaft member can be configured to engage a device retrieval system of the transcatheter pulmonary flow reduction device. The device retrieval system, for example, can extend proximally from the transcatheter pulmonary flow reduction device. The catheter system of the third aspect optionally can further comprise an implant interface member being disposed at the distal shaft end region of the delivery shaft member and being configured to engage the device retrieval system of the transcatheter pulmonary flow reduction device. Additionally and/or alternatively, the implant interface member can include a flexible coupler member extending distally from the distal shaft end region of the delivery shaft member and can include a raised member being configured to engage a retention opening defined by the device retrieval system of the transcatheter pulmonary flow reduction device. The raised member, for instance, can be configured to be disposed within the retention opening defined by the device retrieval system for coupling the transcatheter pulmonary flow reduction device with the distal shaft end region of the delivery shaft member. In selected embodiments, the delivery shaft member can comprise an elongated annular collar body with proximal and distal collar end regions and defining an axial central collar channel, the flexible coupler member can be biased toward a longitudinal axis of the central collar channel defined by the annular collar body and/or a guide wire can be configured to be advanced within the central collar channel for engaging the flexible coupler member and disposing the raised member within the retention opening defined by the device retrieval system.

The guide wire, in selected embodiments, can be configured to be retracted within the central collar channel for disengaging the flexible coupler member and retracting the raised member from the retention opening defined by the device retrieval system. The delivery shaft member, for example, can comprise a micro-threaded deployment rod system, and/or the flexible coupler member can comprise a micro-threaded deployment rod system for cooperating with the micro-threaded deployment rod system. The micro-threaded deployment rod system can define one or more external threads, and/or the micro-threaded deployment rod system can define one or more internal threads for cooperating with the one or more internal threads of the micro-threaded deployment rod system.

In selected embodiments of the catheter system of the third aspect, the delivery shaft member can be configured for deploying the transcatheter pulmonary flow reduction device in the first stable expanded state. The distal shaft end region of the delivery shaft member, for example, can be configured to be disposed within the internal channel defined by the transcatheter pulmonary flow reduction device. In selected embodiments, the catheter system of the third aspect can further comprise an implant expansion system being expandable from an unexpanded state to an expanded state, wherein the implant expansion system can be configured to be disposed within the internal channel defined by the transcatheter pulmonary flow reduction device and to radially expand the transcatheter pulmonary flow reduction device to the first stable expanded state. The transcatheter pulmonary flow reduction device optionally can be configured to self-expand to the first stable expanded state.

In selected embodiments of the catheter system of the third aspect, the delivery shaft member can comprise a hypotube system and/or a braided microcatheter system.

In accordance with a fourth aspect disclosed herein, there is set forth a catheter system (or means) for re-expanding a deployed transcatheter pulmonary flow reduction device for adjusting blood flow through a pulmonary artery of a patient. The catheter system of the fourth aspect, for example, can comprise a catheter system (or means) for re-expanding the transcatheter pulmonary flow reduction device of the first aspect. Additionally and/or alternatively, the catheter system of the third aspect can comprise a catheter system for re-expanding a transcatheter pulmonary flow reduction device being deployed in pulmonary artery of a patient, being in a first stable expanded state and defining an internal channel with an internal cross-section that can comprise a predetermined first cross-section being less than a cross-section of the pulmonary artery for restricting blood flow through the pulmonary artery and can comprise:
a flexible re-expansion shaft member having a distal shaft end region; and
an implant expansion system being disposed at the distal shaft end region of the re-expansion shaft member and being configured to be disposed within the internal channel defined by the transcatheter pulmonary flow reduction device and radially expand from an unexpanded state to an expanded state,
wherein the implant expansion system can be configured to transition the transcatheter pulmonary flow reduction device from the first stable expanded state to a second stable expanded state in which the internal cross-section can comprise a predetermined second cross-section that is less than the cross-section of the pulmonary artery and that is greater than the predetermined first cross-section for adjusting the restricted blood flow through the pulmonary artery via the transcatheter pulmonary flow reduction device.

In selected embodiments of the catheter system of the fourth aspect, the transcatheter pulmonary flow reduction device can comprise a flexible device frame having an annular waist region in axial alignment between an annular proximal frame end region and an annular distal frame end region and being in an implantation state for facilitating insertion into the pulmonary artery of the patient, the annular proximal frame end region, the annular waist region and the annular distal frame end region each defining an external periphery and cooperating to define the internal channel extending from the annular proximal frame end region to the annular distal frame end region. The distal shaft end region of the re-expansion shaft member, for instance, can be configured to engage the annular proximal frame end region of the device frame. The device frame of the deployed transcatheter pulmonary flow reduction device optionally can be configured to be radially re-expanded from the first stable expanded state to the second stable expanded state with the internal cross-section of the annular waist region being further expanded to the predetermined second cross-section.

In selected embodiments of the catheter system of the fourth aspect, the distal shaft end region of the re-expansion shaft member can be configured to engage a device retrieval system of the transcatheter pulmonary flow reduction device. The device retrieval system, for example, can extend proximally from the transcatheter pulmonary flow reduction device. In selected embodiments, the catheter system of the fourth aspect can further comprise an implant interface member being disposed at the distal shaft end region of the re-expansion shaft member and being configured to engage the device retrieval system of the transcatheter pulmonary flow reduction device. The implant interface member, for example, can include a flexible coupler member extending distally from the distal shaft end region of the re-expansion shaft member and can include a raised member being configured to engage a retention opening defined by the device retrieval system of the transcatheter pulmonary flow reduction device.

The raised member optionally can be configured to be disposed within the retention opening defined by the device retrieval system for coupling the transcatheter pulmonary flow reduction device with the distal shaft end region of the re-expansion shaft member. Additionally and/or alternatively, the re-expansion shaft member can comprise an elongated annular collar body with proximal and distal collar end regions and defining an axial central collar channel, the flexible coupler member can be biased toward a longitudinal axis of the central collar channel defined by the annular collar body and/or a guide wire can be configured to be advanced within the central collar channel for engaging the flexible coupler member and disposing the raised member within the retention opening defined by the device retrieval system. The guide wire, for example, can be configured to be retracted within the central collar channel for disengaging the flexible coupler member and retracting the raised member from the retention opening defined by the device retrieval system. In selected embodiments, the re-expansion shaft member can comprise a micro-threaded deployment rod system, and/or the flexible coupler member can comprise a micro-threaded deployment rod system for cooperating with the micro-threaded deployment rod system. The micro-threaded deployment rod system optionally can define one or more external threads, and/or the micro-threaded deployment rod system optionally can define one or more internal threads for cooperating with the one or more internal threads of the micro-threaded deployment rod system.

In selected embodiments of the catheter system of the fourth aspect, the re-expansion shaft member can comprise a hypotube system and/or a braided microcatheter system.

In accordance with a fifth aspect disclosed herein, there is set forth a catheter system (or means) for recapturing a deployed transcatheter pulmonary flow reduction device for adjusting blood flow through a pulmonary artery of a patient. The catheter system of the fifth aspect, for example, can comprise a catheter system (or means) for recapturing the transcatheter pulmonary flow reduction device of the first aspect. Additionally and/or alternatively, the catheter system of the fifth aspect can comprise a catheter system for recapturing a transcatheter pulmonary flow reduction device being deployed in pulmonary artery of a patient and defining an internal channel with an internal cross-section with a predetermined cross-section being less than a cross-section of the pulmonary artery for restricting blood flow through the pulmonary artery and can comprise:
a flexible recapturing shaft member having a distal shaft end region; and/or
an implant interface member being disposed at the distal shaft end region of the recapturing shaft member and being configured to engage the transcatheter pulmonary flow reduction device,
wherein the catheter system can be configured to recapture the transcatheter pulmonary flow reduction device deployed in the pulmonary artery of the patient.

In selected embodiments of the catheter system of the fifth aspect, the transcatheter pulmonary flow reduction device can comprise a flexible device frame having an annular waist region in axial alignment between an annular proximal frame end region and an annular distal frame end region and being in an implantation state for facilitating insertion into the pulmonary artery of the patient, the annular proximal frame end region, the annular waist region and the annular distal frame end region each defining an external periphery and cooperating to define the internal channel extending from the annular proximal frame end region to the annular distal frame end region. The implant interface member, for example, can be configured to engage the annular proximal frame end region of the device frame. The device frame optionally can be configured to be disposed in a first stable expanded state with the external periphery of the annular proximal frame end region and the external periphery of the annular distal frame end region each being adapted to engage the pulmonary artery and with the internal channel of the annular waist region being adapted to radially expand to the predetermined first cross-section. Additionally and/or alternatively, the device frame can be configured to be disposed in a second stable expanded state with the internal cross-section of the annular waist region being further expanded to the predetermined second cross-section.

In selected embodiments of the catheter system of the fifth aspect, the implant interface member can be configured to engage a device retrieval system of the transcatheter pulmonary flow reduction device. The device retrieval system, for example, can extend proximally from the transcatheter pulmonary flow reduction device. The implant interface member optionally can include a flexible coupler member extending distally from the distal shaft end region of the recapturing shaft member and can include a raised member being configured to engage a retention opening defined by the device retrieval system of the transcatheter pulmonary flow reduction device. Additionally and/or alternatively, the raised member can be configured to be disposed within the retention opening defined by the device retrieval system for coupling the transcatheter pulmonary flow reduction device with the distal shaft end region of the recapturing shaft member.

In selected embodiments, the recapturing shaft member can comprise an elongated annular collar body with proximal and distal collar end regions and defining an axial central collar channel, the flexible coupler member can be biased toward a longitudinal axis of the central collar channel defined by the annular collar body and/or a guide wire can be configured to be advanced within the central collar channel for engaging the flexible coupler member and disposing the raised member within the retention opening defined by the device retrieval system. The guide wire, for example, can be configured to be retracted within the central collar channel for disengaging the flexible coupler member and retracting the raised member from the retention opening defined by the device retrieval system. Additionally and/or alternatively, the recapturing shaft member can comprise a micro-threaded deployment rod system, and/or the flexible coupler member can comprise a micro-threaded deployment rod system for cooperating with the micro-threaded deployment rod system. The micro-threaded deployment rod system, for example, can define one or more external threads, and/or the micro-threaded deployment rod system can define one or more internal threads for cooperating with the one or more internal threads of the micro-threaded deployment rod system.

In selected embodiments of the catheter system of the fifth aspect, the recapturing shaft member can comprise a hypotube system and/or a braided microcatheter system.

In accordance with a sixth aspect disclosed herein, there is set forth a catheter system (or means) for repositioning a deployed transcatheter pulmonary flow reduction device for adjusting blood flow through a pulmonary artery of a patient. The catheter system of the sixth aspect, for example, can comprise a catheter system (or means) for repositioning the transcatheter pulmonary flow reduction device of the first aspect. Additionally and/or alternatively, the catheter system of the sixth aspect can comprise a catheter system for repositioning a transcatheter pulmonary flow reduction device being deployed in pulmonary artery of a patient and defining an internal channel with an internal cross-section with a predetermined cross-section being less than a cross-section of the pulmonary artery for restricting blood flow through the pulmonary artery and can comprise:
a flexible repositioning shaft member having a distal shaft end region; and/or
an implant interface member being disposed at the distal shaft end region of the repositioning shaft member and being configured to engage the transcatheter pulmonary flow reduction device,
wherein the catheter system can be configured to reposition the deployed transcatheter pulmonary flow reduction device within the pulmonary artery of the patient.

In selected embodiments of the catheter system of the sixth aspect, the transcatheter pulmonary flow reduction device can comprise a flexible device frame having an annular waist region in axial alignment between an annular proximal frame end region and an annular distal frame end region and being in an implantation state for facilitating insertion into the pulmonary artery of the patient, the annular proximal frame end region, the annular waist region and the annular distal frame end region each defining an external periphery and cooperating to define the internal channel extending from the annular proximal frame end region to the annular distal frame end region. The implant interface member, for example, can be configured to engage the annular proximal frame end region of the device frame. The device frame optionally can be configured to be disposed in a first stable expanded state with the external periphery of the annular proximal frame end region and the external periphery of the annular distal frame end region each being adapted to engage the pulmonary artery and with the internal channel of the annular waist region being adapted to radially expand to the predetermined first cross-section. Additionally and/or alternatively, the device frame can be configured to be disposed in a second stable expanded state with the internal cross-section of the annular waist region being further expanded to the predetermined second cross-section.

In selected embodiments of the catheter system of the sixth aspect, the implant interface member can be configured to engage a device retrieval system of the transcatheter pulmonary flow reduction device. The device retrieval system, for example, can extend proximally from the transcatheter pulmonary flow reduction device. The implant interface member can include an optional flexible coupler member extending distally from the distal shaft end region of the repositioning shaft member and/or can include a raised member being configured to engage a retention opening defined by the device retrieval system of the transcatheter pulmonary flow reduction device. The raised member, for example, can be configured to be disposed within the retention opening defined by the device retrieval system for coupling the transcatheter pulmonary flow reduction device with the distal shaft end region of the repositioning shaft member. Additionally and/or alternatively, the repositioning shaft member can comprise an elongated annular collar body with proximal and distal collar end regions and defining an axial central collar channel, the flexible coupler member can be biased toward a longitudinal axis of the central collar channel defined by the annular collar body, and/or a guide wire can be configured to be advanced within the central collar channel for engaging the flexible coupler member and disposing the raised member within the retention opening defined by the device retrieval system.

The guide wire, in selected embodiments, can be configured to be retracted within the central collar channel for disengaging the flexible coupler member and retracting the raised member from the retention opening defined by the device retrieval system. The repositioning shaft member optionally can comprise a micro-threaded deployment rod system, and/or the flexible coupler member can comprise a micro-threaded deployment rod system for cooperating with the micro-threaded deployment rod system. Additionally and/or alternatively, the micro-threaded deployment rod system can define one or more external threads, and the micro-threaded deployment rod system can define one or more internal threads for cooperating with the one or more internal threads of the micro-threaded deployment rod system.

In selected embodiments of the catheter system of the sixth aspect, the repositioning shaft member can comprise a hypotube system and/or a braided microcatheter system.

In accordance with a seventh aspect disclosed herein, there is set forth a catheter system (or means) for retrieving a deployed transcatheter pulmonary flow reduction device for adjusting blood flow through a pulmonary artery of a patient. The catheter system of the seventh aspect, for example, can comprise a catheter system (or means) for retrieving the transcatheter pulmonary flow reduction device of the first aspect. Additionally and/or alternatively, the catheter system of the seventh aspect can comprise a catheter system for retrieving a transcatheter pulmonary flow reduction device being deployed in pulmonary artery of a patient and defining an internal channel with an internal cross-section with a predetermined cross-section being less than a cross-section of the pulmonary artery for restricting blood flow through the pulmonary artery and can comprise:
a flexible retrieving shaft member having a distal shaft end region; and/or
an implant interface member being disposed at the distal shaft end region of the retrieving shaft member and being configured to engage the transcatheter pulmonary flow reduction device,
wherein the catheter system can be configured to retrieve the deployed transcatheter pulmonary flow reduction device from the pulmonary artery of the patient.

In selected embodiments of the catheter system of the seventh aspect, the transcatheter pulmonary flow reduction device can comprise a flexible device frame having an annular waist region in axial alignment between an annular proximal frame end region and an annular distal frame end region and being in an implantation state for facilitating insertion into the pulmonary artery of the patient, the annular proximal frame end region, the annular waist region and the annular distal frame end region each defining an external periphery and cooperating to define the internal channel extending from the annular proximal frame end region to the annular distal frame end region. The implant interface member, for example, can be configured to engage the annular proximal frame end region of the device frame. The device frame optionally can be configured to be disposed in a first stable expanded state with the external periphery of the annular proximal frame end region and the external periphery of the annular distal frame end region each being adapted to engage the pulmonary artery and with the internal channel of the annular waist region being adapted to radially expand to the predetermined first cross-section. Additionally and/or alternatively, the device frame can be configured to be disposed in a second stable expanded state with the internal cross-section of the annular waist region being further expanded to the predetermined second cross-section.

In selected embodiments of the catheter system of the seventh aspect, the implant interface member can be configured to engage a device retrieval system of the transcatheter pulmonary flow reduction device. The device retrieval system, for example, can extend proximally from the transcatheter pulmonary flow reduction device. The implant interface member optionally can include a flexible coupler member extending distally from the distal shaft end region of the retrieving shaft member and can include a raised member being configured to engage a retention opening defined by the device retrieval system of the transcatheter pulmonary flow reduction device. Additionally and/or alternatively, the raised member can be configured to be disposed within the retention opening defined by the device retrieval system for coupling the transcatheter pulmonary flow reduction device with the distal shaft end region of the retrieving shaft member.

The retrieving shaft member, in selected embodiment, can comprise an elongated annular collar body with proximal and distal collar end regions and defining an axial central collar channel, the flexible coupler member can be biased toward a longitudinal axis of the central collar channel defined by the annular collar body, and/or a guide wire can be configured to be advanced within the central collar channel for engaging the flexible coupler member and disposing the raised member within the retention opening defined by the device retrieval system. The guide wire, for example, can be configured to be retracted within the central collar channel for disengaging the flexible coupler member and retracting the raised member from the retention opening defined by the device retrieval system. The retrieving shaft member optionally can comprise a micro-threaded deployment rod system, and the flexible coupler member optionally can comprise a micro-threaded deployment rod system for cooperating with the micro-threaded deployment rod system. Additionally and/or alternatively, the micro-threaded deployment rod system can define one or more external threads, and/or the micro-threaded deployment rod system can define one or more internal threads for cooperating with the one or more internal threads of the micro-threaded deployment rod system.

In selected embodiments of the catheter system of the seventh aspect, the retrieving shaft member can comprise a hypotube system and/or a braided microcatheter system.

In accordance with an eighth aspect disclosed herein, there is set forth a catheter system (or means) for recovering a deployed transcatheter pulmonary flow reduction device for adjusting blood flow through a pulmonary artery of a patient. The catheter system of the eighth aspect, for example, can comprise a catheter system (or means) for recovering the transcatheter pulmonary flow reduction device of the first aspect. Additionally and/or alternatively, the catheter system of the eighth aspect can comprise a catheter system for recovering a transcatheter pulmonary flow reduction device being deployed in pulmonary artery of a patient and defining an internal channel with an internal cross-section with a predetermined cross-section being less than a cross-section of the pulmonary artery for restricting blood flow through the pulmonary artery and can comprise:
a flexible recovery shaft member having a distal shaft end region; and
an implant interface member being disposed at the distal shaft end region of the recovery shaft member and being configured to engage the transcatheter pulmonary flow reduction device,
wherein the catheter system can be configured to recover the deployed transcatheter pulmonary flow reduction device within the pulmonary artery for removal from the patient.

In selected embodiments of the catheter system of the eighth aspect, the transcatheter pulmonary flow reduction device can comprise a flexible device frame having an annular waist region in axial alignment between an annular proximal frame end region and an annular distal frame end region and being in an implantation state for facilitating insertion into the pulmonary artery of the patient, the annular proximal frame end region, the annular waist region and the annular distal frame end region each defining an external periphery and cooperating to define the internal channel extending from the annular proximal frame end region to the annular distal frame end region. The implant interface member, for instance, can be configured to engage the annular proximal frame end region of the device frame. The device frame optionally can be configured to be disposed in a first stable expanded state with the external periphery of the annular proximal frame end region and the external periphery of the annular distal frame end region each being adapted to engage the pulmonary artery and with the internal channel of the annular waist region being adapted to radially expand to the predetermined first cross-section. Additionally and/or alternatively, the device frame can be configured to be disposed in a second stable expanded state with the internal cross-section of the annular waist region being further expanded to the predetermined second cross-section.

In selected embodiments of the catheter system of the eighth aspect, the implant interface member can be configured to engage a device recovery system of the transcatheter pulmonary flow reduction device. The device recovery system, for example, can extend proximally from the transcatheter pulmonary flow reduction device. The implant interface member optionally can include a flexible coupler member extending distally from the distal shaft end region of the recovery shaft member and can include a raised member being configured to engage a retention opening defined by the device recovery system of the transcatheter pulmonary flow reduction device. Additionally and/or alternatively, the raised member can be configured to be disposed within the retention opening defined by the device recovery system for coupling the transcatheter pulmonary flow reduction device with the distal shaft end region of the recovery shaft member.

The recovery shaft member, in selected embodiments, can comprise an elongated annular collar body with proximal and distal collar end regions and defining an axial central collar channel, the flexible coupler member can be biased toward a longitudinal axis of the central collar channel defined by the annular collar body, and/or a guide wire can be configured to be advanced within the central collar channel for engaging the flexible coupler member and disposing the raised member within the retention opening defined by the device recovery system. The guide wire, for example, can be configured to be retracted within the central collar channel for disengaging the flexible coupler member and retracting the raised member from the retention opening defined by the device recovery system.

Additionally and/or alternatively, the recovery shaft member can comprise a micro-threaded deployment rod system, and/or the flexible coupler member can comprise a micro-threaded deployment rod system for cooperating with the micro-threaded deployment rod system. The micro-threaded deployment rod system, for example, can define one or more external threads, and/or the micro-threaded deployment rod system can define one or more internal threads for cooperating with the one or more internal threads of the micro-threaded deployment rod system.

In selected embodiments of the catheter system of the eighth aspect, the recovery shaft member can comprise a hypotube system and/or a braided microcatheter system.

In accordance with a ninth aspect disclosed herein, there is set forth a method for deploying a transcatheter pulmonary flow reduction device for adjusting blood flow through a pulmonary artery of a patient. The method of the ninth aspect, for example, can comprise a method for deploying the transcatheter pulmonary flow reduction device of the first aspect.

Additionally and/or alternatively, the method of the ninth aspect can comprise deploying the transcatheter pulmonary flow reduction device via a surgical procedure and/or via the catheter system of the third aspect, without limitation. In selected embodiments, the method of the ninth aspect can comprise:
introducing a flexible device frame into the pulmonary artery of the patient, the device frame having an annular waist region in axial alignment between an annular proximal frame end region and an annular distal frame end region and being in an implantation state, the annular proximal frame end region, the annular waist region and the annular distal frame end region each defining an external periphery and cooperating to define an internal channel extending from the annular proximal frame end region to the annular distal frame end region; and
radially expanding the device frame within the pulmonary artery from the implantation state to a first stable expanded state with the external periphery of the annular proximal frame end region and the external periphery of the annular distal frame end region each being adapted to engage the pulmonary artery and with the internal channel of the annular waist region having an internal cross-section being adapted to radially expand to a predetermined first cross-section that is less than a cross-section of the pulmonary artery for restricting the blood flow through the pulmonary artery via the device frame,
wherein the deployed device frame can be configured to be subsequently radially re-expanded from the first stable expanded state to a second stable expanded state with the internal cross-section of the annular waist region being further expanded to a predetermined second cross-section that is less than the cross-section of the pulmonary artery and that is greater than the predetermined first cross-section for adjusting the restricted blood flow through the pulmonary artery via the device frame.

In accordance with a tenth aspect disclosed herein, there is set forth a method for delivering a transcatheter pulmonary flow reduction device for adjusting blood flow through a pulmonary artery of a patient. The method of the tenth aspect, for example, can comprise a method for delivering the transcatheter pulmonary flow reduction device of the first aspect. Additionally and/or alternatively, the method of the tenth aspect can comprise delivering the transcatheter pulmonary flow reduction device via a surgical procedure and/or via the catheter system of the third aspect, without limitation. In selected embodiments, the method of the tenth aspect can comprise a method for delivering a transcatheter pulmonary flow reduction device defining an internal channel with a controllable internal cross-section for adjusting blood flow through a pulmonary artery of a patient and can comprise:
introducing a flexible delivery shaft member into the pulmonary artery of the patient, the delivery shaft member having a distal shaft end region for engaging the transcatheter pulmonary flow reduction device,
delivering the transcatheter pulmonary flow reduction device to the pulmonary artery of the patient for deployment in a first stable expanded state in which the internal cross-section can comprise a predetermined first cross-section that is less than a cross-section of the pulmonary artery for restricting the blood flow through the pulmonary artery via the transcatheter pulmonary flow reduction device, and
wherein the deployed transcatheter pulmonary flow reduction device can be configured to subsequently transition from the first stable expanded state to a second stable expanded state in which the internal cross-section can comprise a predetermined second cross-section that is less than the cross-section of the pulmonary artery and that is greater than the predetermined first cross-section for adjusting the restricted blood flow through the pulmonary artery via the transcatheter pulmonary flow reduction device.

In accordance with an eleventh aspect disclosed herein, there is set forth a method for re-expanding a deployed transcatheter pulmonary flow reduction device for adjusting blood flow through a pulmonary artery of a patient. The method of the eleventh aspect, for example, can comprise a method for re-expanding the transcatheter pulmonary flow reduction device of the first aspect. Additionally and/or alternatively, the method of the eleventh aspect can comprise re-expanding the transcatheter pulmonary flow reduction device via a surgical procedure and/or via the catheter system of the fourth aspect, without limitation. In selected embodiments, the method of the eleventh aspect can include a method for re-expanding a transcatheter pulmonary flow reduction device being deployed in pulmonary artery of a patient, being in a first stable expanded state and defining an internal channel with an internal cross-section that comprises a predetermined first cross-section being less than a cross-section of the pulmonary artery for restricting blood flow through the pulmonary artery and can comprise:
introducing a flexible re-expansion shaft member having a distal shaft end region into the pulmonary artery of the patient, an implant expansion system being disposed at the distal shaft end region of the re-expansion shaft member;
disposing the implant expansion system within the internal channel defined by the transcatheter pulmonary flow reduction device; and
radially expanding the implant expansion system from an unexpanded state to an expanded state,
wherein the implant expansion system can be configured to transition the transcatheter pulmonary flow reduction device from the first stable expanded state to a second stable expanded state in which the internal cross-section can comprise a predetermined second cross-section that is less than the cross-section of the pulmonary artery and that is greater than the predetermined first cross-section for adjusting the restricted blood flow through the pulmonary artery via the transcatheter pulmonary flow reduction device.

In accordance with a twelfth aspect disclosed herein, there is set forth a method for recapturing a deployed transcatheter pulmonary flow reduction device for adjusting blood flow through a pulmonary artery of a patient. The method of the twelfth aspect, for example, can comprise a method for recapturing the transcatheter pulmonary flow reduction device of the first aspect. Additionally and/or alternatively, the method of the twelfth aspect can comprise recapturing the transcatheter pulmonary flow reduction device via a surgical procedure and/or via the catheter system of the fifth aspect, without limitation. In selected embodiments, the method of the twelfth aspect can include a method for recapturing a transcatheter pulmonary flow reduction device being deployed in pulmonary artery of a patient and defining an internal channel with an internal cross-section with a predetermined cross-section being less than a cross-section of the pulmonary artery for restricting blood flow through the pulmonary artery and can comprise:
introducing a flexible recapturing shaft member having a distal shaft end region into the pulmonary artery of the patient, an implant interface member being disposed at the distal shaft end region of the recapturing shaft member; and
engaging the transcatheter pulmonary flow reduction device via the implant interface member,
wherein the implant interface member can be configured to recapture the transcatheter pulmonary flow reduction device deployed in the pulmonary artery of the patient.

In accordance with a thirteenth aspect disclosed herein, there is set forth a method for repositioning a deployed transcatheter pulmonary flow reduction device for adjusting blood flow through a pulmonary artery of a patient. The method of the thirteenth aspect, for example, can comprise a method for repositioning the transcatheter pulmonary flow reduction device of the first aspect. Additionally and/or alternatively, the method of the thirteenth aspect can comprise repositioning the transcatheter pulmonary flow reduction device via a surgical procedure and/or via the catheter system of the sixth aspect, without limitation. In selected embodiments, the method of the thirteenth aspect can include a method for repositioning a transcatheter pulmonary flow reduction device being deployed in pulmonary artery of a patient and defining an internal channel with an internal cross-section with a predetermined cross-section being less than a cross-section of the pulmonary artery for restricting blood flow through the pulmonary artery and can comprise:
introducing a flexible repositioning shaft member having a distal shaft end region into the pulmonary artery of the patient, an implant interface member being disposed at the distal shaft end region of the repositioning shaft member; and
engaging the transcatheter pulmonary flow reduction device via the implant interface member,
wherein the implant interface member can be configured to reposition the deployed transcatheter pulmonary flow reduction device within the pulmonary artery of the patient.

In accordance with a fourteenth aspect disclosed herein, there is set forth a method for retrieving a deployed transcatheter pulmonary flow reduction device for adjusting blood flow through a pulmonary artery of a patient. The method of the fourteenth aspect, for example, can comprise a method for retrieving the transcatheter pulmonary flow reduction device of the first aspect. Additionally and/or alternatively, the method of the fourteenth aspect can comprise retrieving the transcatheter pulmonary flow reduction device via a surgical procedure and/or via the catheter system of the seventh aspect, without limitation. In selected embodiments, the method of the fourteenth aspect can include a method for retrieving a transcatheter pulmonary flow reduction device being deployed in pulmonary artery of a patient and defining an internal channel with an internal cross-section with a predetermined cross-section being less than a cross-section of the pulmonary artery for restricting blood flow through the pulmonary artery and can comprise:
introducing a flexible retrieving shaft member having a distal shaft end region into the pulmonary artery of the patient, an implant interface member being disposed at the distal shaft end region of the retrieving shaft member; and
engaging the transcatheter pulmonary flow reduction device via the implant interface member,
wherein the implant interface member can be configured to retrieve the deployed transcatheter pulmonary flow reduction device from the pulmonary artery of the patient.

In accordance with a fifteenth aspect disclosed herein, there is set forth a method for recovering a deployed transcatheter pulmonary flow reduction device for adjusting blood flow through a pulmonary artery of a patient. The method of the fifteenth aspect, for example, can comprise a method for recovering the transcatheter pulmonary flow reduction device of the first aspect. Additionally and/or alternatively, the method of the fourteenth aspect can comprise recovering the transcatheter pulmonary flow reduction device via a surgical procedure and/or via the catheter system of the eighth aspect, without limitation. In selected embodiments, the method of the fifteenth aspect can include a method for recovering a transcatheter pulmonary flow reduction device being deployed in pulmonary artery of a patient and defining an internal channel with an internal cross-section with a predetermined cross-section being less than a cross-section of the pulmonary artery for restricting blood flow through the pulmonary artery and can comprise:
introducing a flexible recovery shaft member having a distal shaft end region into the pulmonary artery of the patient, an implant interface member being disposed at the distal shaft end region of the recovery shaft member; and
engaging the transcatheter pulmonary flow reduction device via the recovery shaft member,
wherein the implant interface member can be configured to recover the deployed transcatheter pulmonary flow reduction device within the pulmonary artery for removal from the patient.

### BRIEF DESCRIPTION OF THE DRAWINGS

FIG. 1 is a top-level diagram illustrating an exemplary embodiment of a transcatheter pulmonary flow reduction device.

FIG. 2 is a detail diagram illustrating an exemplary embodiment of the transcatheter pulmonary flow reduction device of FIG. 1, wherein the transcatheter pulmonary flow reduction device is disposed in a deployed configuration.

FIG. 3A is a detail diagram illustrating an exemplary embodiment of the transcatheter pulmonary flow reduction device of FIG. 2, wherein the transcatheter pulmonary flow reduction device comprises a proximal device frame lobe with a first annular arrangement of device frame struts and a distal device frame lobe with a second annular arrangement of device frame struts.

FIG. 3B is a detail diagram illustrating an exemplary embodiment of strut patterns and cell configurations of the transcatheter pulmonary flow reduction device of FIG. 3A.

FIGS. 4A-D are top-level diagrams illustrating an exemplary embodiment of a method for implanting the transcatheter pulmonary flow reduction device of FIG. 2 in a pulmonary artery of a patient via a delivery catheter system.

FIGS. 5A-E are top-level diagrams illustrating an exemplary embodiment of a method for subsequently expanding an internal channel defined by the implanted transcatheter pulmonary flow reduction device of FIGS. 4A-D for increasing blood flow within the pulmonary artery.

FIG. 6A is a detail diagram illustrating an exemplary alternative embodiment of the transcatheter pulmonary flow reduction device of FIG. 2, wherein the transcatheter pulmonary flow reduction device includes a device retrieval system.

FIG. 6B is a detail diagram illustrating an exemplary embodiment of strut patterns and cell configurations of the transcatheter pulmonary flow reduction device of FIG. 6A.

FIG. 7A is a detail diagram illustrating an exemplary embodiment of a device retrieval member of the device retrieval system of FIGS. 6A-B.

FIG. 7B is a detail diagram illustrating an exemplary alternative embodiment of the device retrieval member of FIG. 7A, wherein the device retrieval member includes a first device engagement system with a square profile.

FIG. 7C is a detail diagram illustrating another exemplary alternative embodiment of the device retrieval member of FIG. 7A, wherein the device retrieval member includes a second device engagement system with a T-shaped profile.

FIG. 7D is a detail diagram illustrating yet another exemplary alternative embodiment of the device retrieval member of FIG. 7A, wherein the device retrieval member includes a third device engagement system with a circular profile.

FIG. 8A is a detail diagram illustrating an exemplary alternative embodiment of the transcatheter pulmonary flow reduction device of FIGS. 2, 3A-B and 6A-B, wherein the device retrieval system comprises a plurality of device retrieval members with distal retrieval member end regions that converge to form a common capture point.

FIG. 8B is a detail diagram illustrating an exemplary embodiment of strut patterns and cell configurations of the transcatheter pulmonary flow reduction device of FIG. 8A.

FIG. 9A is a detail diagram illustrating another exemplary alternative embodiment of the transcatheter pulmonary flow reduction device of FIGS. 2, 3A-B and 6A-B, wherein the device retrieval system comprises a plurality of device retrieval members with distal retrieval member end regions that converge at a coupling device for engaging an implant retrieval system.

FIG. 9B is a detail diagram illustrating an exemplary embodiment of strut patterns and cell configurations of the transcatheter pulmonary flow reduction device of FIG. 9A.

FIG. 10A is a detail diagram illustrating yet another exemplary alternative embodiment of the transcatheter pulmonary flow reduction device of FIGS. 2, 3A-B and 6A-B, wherein the device retrieval system defines at least one enlarged retrieval system cell.

FIG. 10B is a detail diagram illustrating an exemplary embodiment of strut patterns and cell configurations of the transcatheter pulmonary flow reduction device of FIG. 10A.

FIG. 11A is a detail diagram illustrating still another exemplary alternative embodiment of the transcatheter pulmonary flow reduction device of FIGS. 2, 3A-B and 6A-B, wherein the device retrieval system comprises a reduced number of device retrieval members with distal retrieval member end regions that converge at a coupling device for engaging the implant retrieval system.

FIG. 11B is a detail diagram illustrating an exemplary embodiment of strut patterns and cell configurations of the transcatheter pulmonary flow reduction device of FIG. 11A.

FIG. 12A is a detail diagram illustrating a further exemplary alternative embodiment of the transcatheter pulmonary flow reduction device of FIGS. 2, 3A-B and 6A-B, wherein pairs of adjacent distal strut end regions of the transcatheter pulmonary flow reduction device converge to form respective device retrieval members of the device retrieval system with distal retrieval member end regions that extend radially inwardly toward a longitudinal axis of the internal channel defined by the transcatheter pulmonary flow reduction device.

FIG. 12B is a detail diagram illustrating an exemplary embodiment of strut patterns and cell configurations of the transcatheter pulmonary flow reduction device of FIG. 12A.

FIG. 13A is a detail diagram illustrating another further exemplary alternative embodiment of the transcatheter pulmonary flow reduction device of FIGS. 2, 3A-B and 6A-B, wherein pairs of adjacent distal strut end regions of the transcatheter pulmonary flow reduction device converge to form respective device retrieval members of the device retrieval system with distal retrieval member end regions that converge at a coupling device for engaging the implant retrieval system.

FIG. 13B is a detail diagram illustrating an exemplary embodiment of strut patterns and cell configurations of the transcatheter pulmonary flow reduction device of FIG. 13A.

FIG. 14A is a detail diagram illustrating still another further exemplary alternative embodiment of the transcatheter pulmonary flow reduction device of FIGS. 2, 3A-B and 6A-B, wherein pairs of adjacent distal strut end regions of the transcatheter pulmonary flow reduction device converge to form respective shape-angled device retrieval members of the device retrieval system with distal retrieval member end regions that converge at a coupling device for engaging the implant retrieval system.

FIG. 14B is a detail diagram illustrating an exemplary embodiment of strut patterns and cell configurations of the transcatheter pulmonary flow reduction device of FIG. 14A.

FIGS. 15A-B are detail diagrams illustrating an exemplary alternative embodiment of the transcatheter pulmonary flow reduction device of FIGS. 2 and 3A-B, wherein the transcatheter pulmonary flow reduction device includes a cover member for restricting blood flow through the internal channel defined by the transcatheter pulmonary flow reduction device.

FIGS. 16A-B are detail diagrams illustrating an exemplary alternative embodiment of the transcatheter pulmonary flow reduction device of FIGS. 6A-B, wherein the transcatheter pulmonary flow reduction device includes a cover member for restricting blood flow through the internal channel defined by the transcatheter pulmonary flow reduction device.

FIGS. 17A-B are detail diagrams illustrating an exemplary alternative embodiment of the transcatheter pulmonary flow reduction device of FIGS. 11A-B, wherein the transcatheter pulmonary flow reduction device includes a cover member for restricting blood flow through the internal channel defined by the transcatheter pulmonary flow reduction device.

FIGS. 18A-B are detail diagrams illustrating an exemplary alternative embodiment of the transcatheter pulmonary flow reduction device of FIGS. 14A-B, wherein the transcatheter pulmonary flow reduction device includes a cover member for restricting blood flow through the internal channel defined by the transcatheter pulmonary flow reduction device.

FIG. 19 is a detail diagram illustrating an exemplary alternative embodiment of the transcatheter pulmonary flow reduction devices of FIGS. 15A-B, 16A-B, 17A-B and 18A-B, wherein the cover member is configured to restrict a central waist region of the transcatheter pulmonary flow reduction device.

FIG. 20 is a detail diagram illustrating an exemplary alternative embodiment of the transcatheter pulmonary flow reduction devices of FIG. 19, wherein the cover member includes a circumferential cover loop member for constricting the central waist region of the transcatheter pulmonary flow reduction device.

FIG. 21 is a detail diagram illustrating an exemplary alternative embodiment of the transcatheter pulmonary flow reduction devices of FIG. 19, wherein the cover member includes a circumferential expandable band member for constricting the central waist region of the transcatheter pulmonary flow reduction device.

FIG. 22 is a detail diagram illustrating still another exemplary alternative embodiment of the transcatheter pulmonary flow reduction devices of FIG. 19, wherein the cover member includes a circumferential septum member for constricting the central waist region of the transcatheter pulmonary flow reduction device.

FIG. 23 is a top-level diagram illustrating an exemplary embodiment of the delivery catheter system of FIGS. 4A-D, wherein the delivery catheter system comprises a delivery shaft member and an implant interface member for engaging the transcatheter pulmonary flow reduction device.

FIGS. 24A-B are detail diagrams illustrating an exemplary embodiment of the delivery shaft member of FIG. 23.

FIG. 25 is a detail diagram illustrating an exemplary embodiment of the implant interface member of FIG. 23.

FIG. 26 is detail diagram illustrating an exemplary alternative embodiment of the delivery catheter system of FIG. 23, wherein the implant interface member includes an interface engagement device for engaging the device retrieval system of the transcatheter pulmonary flow reduction device.

FIG. 27A is a detail diagram illustrating an exemplary embodiment of the interface engagement device of FIG. 26, wherein the interface engagement device is configured to removably engage at least one engagement opening of the device retrieval system of the transcatheter pulmonary flow reduction device.

FIG. 27B is a detail diagram illustrating an exemplary embodiment of the interface engagement device of FIG. 27A, wherein the interface engagement device is disposed in an expanded coupler state.

FIG. 28 is a detail diagram illustrating an exemplary alternative embodiment of the transcatheter pulmonary flow reduction device of FIGS. 16A-B, wherein transcatheter pulmonary flow reduction device is deployed in a branch pulmonary artery of the patient.

FIG. 29 is a detail diagram illustrating an exemplary embodiment of a collection of transcatheter pulmonary flow reduction devices having respective dimensions.

FIGS. 30A-B are detail diagrams illustrating yet another further exemplary alternative embodiment of the transcatheter pulmonary flow reduction device of FIGS. 2, 3A-B and 6A-B, wherein the device retrieval system comprises a plurality of device retrieval members with distal retrieval member end regions that converge at a coupling device for engaging an implant retrieval system.

FIG. 31 is a detail diagram illustrating an exemplary embodiment of a distal strut end region of the transcatheter pulmonary flow reduction device of FIGS. 2, 3A-B and 6A-B.

FIG. 32 is a detail diagram illustrating an exemplary alternative embodiment of the distal strut end region of FIG. 31, wherein the distal strut end region is configured to engage a vessel wall of the pulmonary artery of the patient.

FIG. 33 is a detail diagram illustrating another exemplary alternative embodiment of the distal strut end region of FIG. 31, wherein the distal strut end region is configured to provide an increased engagement with the vessel wall of the pulmonary artery of the patient.

FIG. 34 is a detail diagram illustrating yet another exemplary alternative embodiment of the distal strut end region of FIG. 31, wherein the distal strut end region is configured to provide a decreased engagement with the vessel wall of the pulmonary artery of the patient.

It should be noted that the figures are not drawn to scale and that elements of similar structures or functions are generally represented by like reference numerals for illustrative purposes throughout the figures. It also should be noted that the figures are only intended to facilitate the description of the preferred embodiments. The figures do not illustrate every aspect of the described embodiments and do not limit the scope of the present disclosure.

### DETAILED DESCRIPTION OF THE PREFERRED EMBODIMENTS

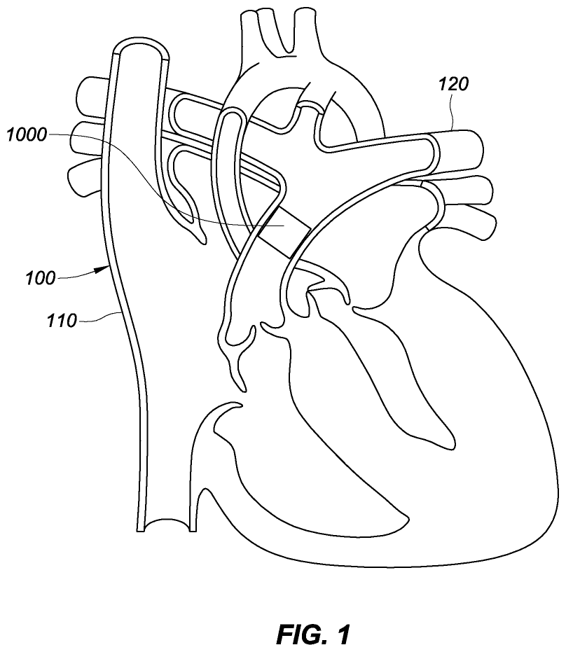

Since currently-available devices and therapies for controlled reduction of pulmonary blood flow are invasive, cannot be adjusted after implantation and are associated with prolonged recovery times, risks of infection, and negative developmental effects, a transcatheter (or transvascular) pulmonary flow reduction device and method that allow a patient with congenital heart disease to reduce an amount of blood flow from the pulmonary artery to the lungs of the patient in a controllable or otherwise adjustable manner can prove desirable and provide a basis for a wide range of applications, such as in treating neonatal, infant, toddler, young children and other pediatric patients who have been diagnosed with single ventricle physiology or other congenital heart conditions necessitating pulmonary flow restriction. This result can be achieved, according to one embodiment disclosed herein, by a transcatheter pulmonary flow reduction device (or implant) 1000 as shown in FIG. 1.

Although many of the embodiments described herein are particularly well suited for neonatal and other pediatric patients due to their low-profile delivery requirements and small vessel compatibility, the transcatheter pulmonary flow reduction device 1000 is not limited to use in infants or children. In various embodiments, the transcatheter pulmonary flow reduction device 1000 may be configured for use in adolescent and/or adult patients requiring vessel blood flow reduction, without limitation.

In adult applications, for example, the transcatheter pulmonary flow reduction device 1000 may be scaled in overall diameter, length, strut thickness and/or radial strength to accommodate larger pulmonary artery dimensions and higher hemodynamic loads, while preserving the same hourglass-shaped flow-restrictive geometry and post-implant adjustability described herein. The ability to provide controlled, adjustable, and optionally retrievable pulmonary flow reduction may be beneficial in adult patients with conditions including, but not limited to, pulmonary hypertension, heart failure with congenital or acquired shunts, postoperative flow imbalance, or other clinical scenarios in which modulation of pulmonary blood flow is desired.

Accordingly, unless expressly stated otherwise, the embodiments of the transcatheter pulmonary flow reduction device 1000 disclosed herein are applicable to neonatal, pediatric, adolescent, and adult patients, and references to neonatal or pediatric use are provided by way of example and not limitation.

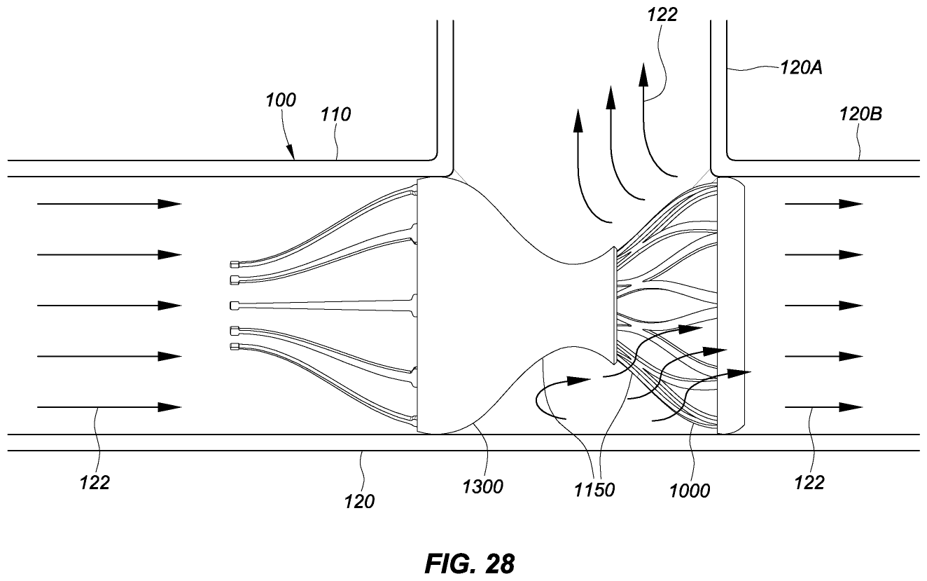

In various embodiments, the transcatheter pulmonary flow reduction device 1000 can be configured for deployment within pulmonary arteries having a range of inner diameters. For neonatal and infant patients, the transcatheter pulmonary flow reduction device 1000 may be sized for use in branch pulmonary arteries 120A, 120B (shown in FIG. 28) having inner diameters ranging from approximately four millimeters to approximately ten millimeters. The transcatheter pulmonary flow reduction device 1000 may be provided in multiple sizes and/or scaled configurations to accommodate this anatomical range while maintaining adequate anchoring, sealing, and flow-restrictive performance. In pediatric, adolescent, and adult applications, the transcatheter pulmonary flow reduction device 1000 may be proportionally scaled to accommodate larger pulmonary artery diameters, while preserving the same hourglass-shaped geometry, central waist flow-restriction, and post-implant adjustability described herein.

In some embodiments, the transcatheter pulmonary flow reduction device 1000 can be configured to be crimped into a sheath or catheter for delivery and retrieval. In certain embodiments, the transcatheter pulmonary flow reduction device 1000 can be capable of crimping to an inner diameter (ID) of less than one millimeter, corresponding approximately to a four French catheter or sheath. In other embodiments, the transcatheter pulmonary flow reduction device 1000 may be configured to crimp to an ID suitable for insertion into a five French catheter or sheath. The crimpable configuration can allow the transcatheter pulmonary flow reduction device 1000 to be temporarily reduced in diameter while maintaining its structural integrity and functional performance upon deployment.

In certain embodiments, the transcatheter pulmonary flow reduction device 1000 may be expanded to its deployed configuration using a balloon-expandable mechanism rather than a self-expanding metallic frame. In such embodiments, the transcatheter pulmonary flow reduction device 1000 may be delivered in a low-profile, unexpanded state and subsequently expanded within the target vessel by inflation of an hourglass-shaped catheter balloon. The balloon may define enlarged proximal and distal regions separated by a narrowed intermediate region such that, upon inflation, the transcatheter pulmonary flow reduction device 1000 can assume an hourglass-shaped geometry that can anchor against the vessel wall while establishing a central flow-restrictive waist. The balloon-expandable transcatheter pulmonary flow reduction device 1000 may comprise plastically deformable metallic materials, polymeric structures, or composite constructions, and may be configured to achieve controlled radial expansion, predictable final geometry, and post-deployment stability without reliance on self-expanding shape-memory behavior.

Although the transcatheter pulmonary flow reduction device 1000 is primarily described herein with reference to pulmonary artery implantation, the disclosed devices and methods are not limited to use within the pulmonary vasculature. In various embodiments, the transcatheter pulmonary flow reduction device 1000 may be deployed within other vessels within the body of the patient 100 where controlled reduction of blood flow is desired. Such locations may include, but are not limited to, systemic arteries, veins, surgically created shunts, grafts, conduits and/or other native or synthetic lumens. The transcatheter pulmonary flow reduction device 1000 may be adapted in size, geometry, material selection, or radial strength to suit the anatomical and hemodynamic requirements of these alternative locations while preserving the same flow-restrictive, adjustable, and optionally retrievable features described herein.

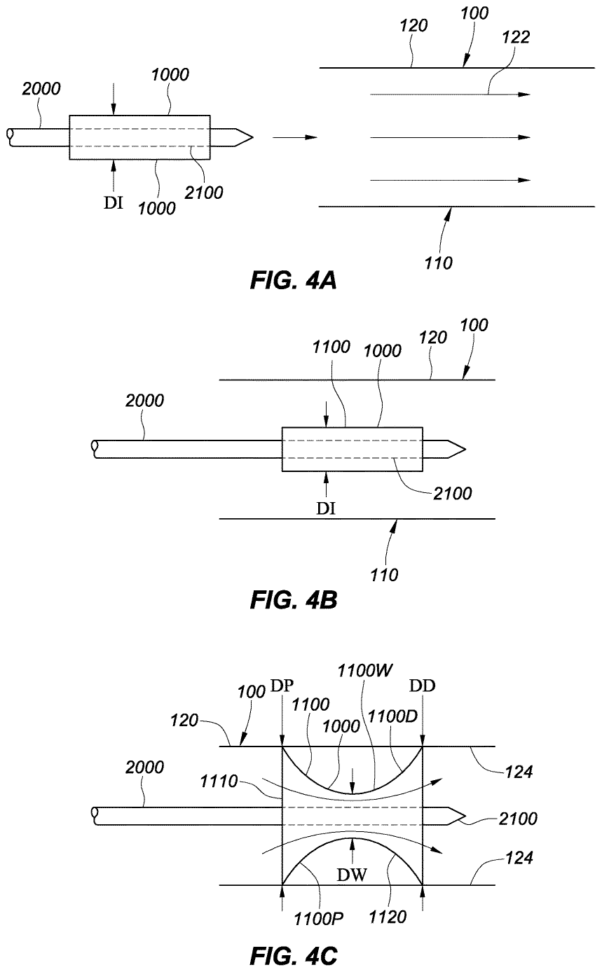

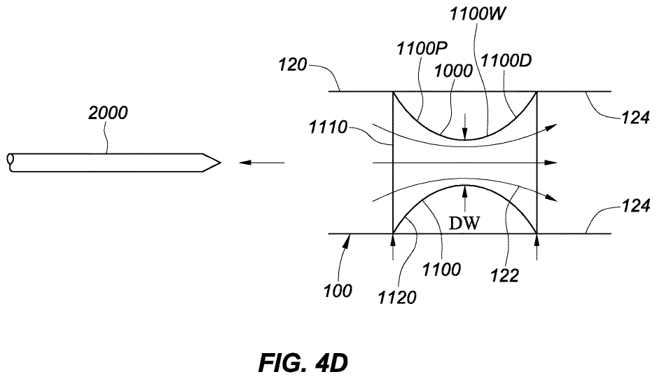

Turning to FIG. 1, the transcatheter pulmonary flow reduction device 1000 is shown as being configured for implantation and deployment within a pulmonary artery 120, such as a selected branch pulmonary artery 120A, 120B (shown in FIG. 28) or other lumen, of a patient 100 with congenital heart disease in a minimally invasive manner. The transcatheter pulmonary flow reduction device 1000, when implanted, can percutaneously reduce (or partially restrict) pulmonary blood flow 122 (shown in FIGS. 4A-D and 5A-E) from a heart 110 to lungs (not shown) of the patient 100 via the pulmonary artery 120. The transcatheter pulmonary flow reduction device 1000 advantageously can comprise an adjustable transcatheter pulmonary flow reduction device for reducing the amount of the pulmonary blood flow 122 in a controllable or otherwise adjustable manner. An amount of flow restriction provided by the transcatheter pulmonary flow reduction device 1000, for example, can be adjusted after implantation of the transcatheter pulmonary flow reduction device 1000 based up on one or more hemodynamic needs of the patient 100. Additionally and/or alternatively, the transcatheter pulmonary flow reduction device 1000 can comprise a removable transcatheter pulmonary flow reduction device that can be later retrieved from the pulmonary artery 120 after deployment.

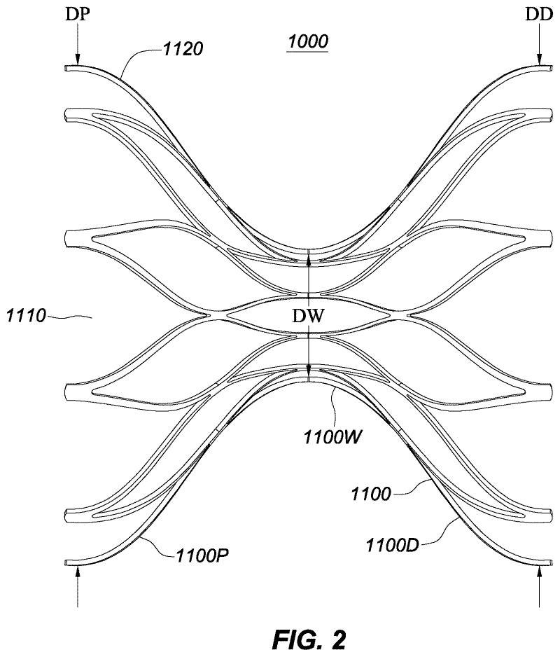

The transcatheter pulmonary flow reduction device 1000, in selected embodiments, can be provided as a part of a pulmonary flow reducer (PFR) system (or means) (not shown). Stated somewhat differently, the pulmonary flow reducer system can comprise a family (or plurality) of the transcatheter pulmonary flow reduction devices 1000 each having respective device sizes, shapes, diameters, cross-sections or other dimensions. The transcatheter pulmonary flow reduction devices 1000 of the pulmonary flow reducer system, for example, can have external peripheries 1120 (shown in FIG. 2) with respective outer diameters. The respective dimensions preferably are different among the transcatheter pulmonary flow reduction devices 1000 for accommodating an anatomical range of neonatal and other pediatric pulmonary arteries. The transcatheter pulmonary flow reduction devices 1000 optionally can be crimped or otherwise compressed to respective initial (or implantation) dimensions DI (shown in FIGS. 4A-D) that are compatible for disposal within a delivery catheter system (or means) 2000 (shown in FIGS. 4A-D) for introduction and implantation in the patient 100. Stated somewhat differently, the transcatheter pulmonary flow reduction devices 1000 can be disposed in an initial (or implantation) state with the implantation dimensions DI such that the transcatheter pulmonary flow reduction device 1000 is configured for disposal on the delivery catheter system 2000 to traverse the patient vasculature and reach the pulmonary artery 120 of the patient 100 for deployment.

As set forth above, the transcatheter pulmonary flow reduction device 1000 can allow a patient 100 with a congenital heart disease to reduce the amount of blood flow 122 (shown in FIGS. 4A-D and 5A-E) from the pulmonary artery 120 to the lungs in an adjustable and otherwise controllable manner. In selected embodiments, the transcatheter pulmonary flow reduction device 1000 can allow the patient 100 with a congenital heart disease, which can lead to increased pulmonary blood flow 122, to achieve a controlled reduction in pulmonary perfusion without a need for surgical pulmonary artery banding. The transcatheter pulmonary flow reduction devices 1000, for example, can be beneficial for neonate and infant patients who present with single ventricle physiology, such as Hypoplastic Left Heart Syndrome, Hypoplastic Right Heart Syndrome, tricuspid atresia or other congenital cardiac anomalies associated with excessive pulmonary blood flow 122.

The transcatheter pulmonary flow reduction device 1000 advantageously can replicate, and/or improve upon, the function of surgical pulmonary artery banding but through a minimally invasive, adjustable and/or removable transcatheter approach. By combining controlled flow restriction, post-implant adjustability, and retrievability in a single device platform, the transcatheter pulmonary flow reduction devices 1000 can address long-standing limitations of existing surgical and transcatheter therapies. These features enable individualized hemodynamic control and significantly reduce procedural risk, recovery time, and morbidity in patients with congenital heart disease requiring pulmonary flow modulation. Although shown and described herein with reference to implantation and deployment in neonates, infants, toddlers, young children and other pediatric patients for purposes of illustration only, the transcatheter pulmonary flow reduction device 1000 can be implanted and deployed in patients of any age or size who have congenital heart disease.

The transcatheter pulmonary flow reduction device 1000 can be provided in any suitable structural arrangement (or configuration). Turning to FIG. 2, for example, the transcatheter pulmonary flow reduction device 1000 is shown as being disposed in a deployed (or expanded) configuration. The transcatheter pulmonary flow reduction device 1000 is illustrated as comprising an hourglass-shaped device frame 1100 with proximal and distal frame end regions 1100P, 1100D. Preferably comprising a flexible device frame, the device frame 1100 can define a central (or internal) channel 1110 and/or an external periphery 1120 of the transcatheter pulmonary flow reduction device 1000.

The external periphery 1120 at the proximal frame end region 1100P of the device frame 1100 can have a predetermined size, shape, diameter, cross-section or other dimension DP; whereas, the external periphery 1120 at the distal frame end region 1100D can have a predetermined size, shape, diameter, cross-section or other dimension DD. The predetermined dimension DP of the proximal frame end region 1100P can be greater than, equal to or less than the predetermined dimension DD of the distal frame end region 1100D. As shown in FIG. 2, the predetermined dimension DP of the proximal frame end region 1100P and the predetermined dimension DD of the distal frame end region 1100D each can be greater than an external size, shape, diameter, cross-section or other dimension of the external periphery 1120 at a central (or intermediate) waist region 1100W of the device frame 1100 being disposed between the proximal and distal frame end regions 1100P, 1100D. The internal channel 1110 of the central waist region 1100W can comprise a flow-restrictive region of the device frame 1100 and can be defined as having an internal size, shape, diameter, cross-section or other dimension DW that can be controllable or otherwise adjustable.

The hourglass configuration of the transcatheter pulmonary flow reduction device 1000 of FIG. 2 can define a first angled taper between the proximal frame end region 1100P and the central waist region 1100W and a second angled taper between the distal frame end region 1100D and the central waist region 1100W. Stated somewhat differently, the external periphery 1120 of the proximal frame end region 1100P and the external periphery 1120 of the central waist region 1100W can define a first frame taper angle; whereas, the external periphery 1120 of the distal frame end region 1100F and the external periphery 1120 of the central waist region 1100W can define a second frame taper angle. The first and second frame taper angles can comprise any suitable angle, such as any predetermined angle within a taper angle range between twenty degrees and eighty degrees, or within any angle subrange of the taper angle range, without limitation. Although preferably comprising uniform angles, the first and second frame taper angles can be different in selected embodiments and/or in selected applications.

The predetermined taper angle, for example, can be selected to improve axial stability of the transcatheter pulmonary flow reduction device 1000 when disposed within the pulmonary artery 120 while minimizing an overall length of the transcatheter pulmonary flow reduction device 1000. The reduced axial footprint can help to prevent jailing of adjacent arterial segments or bifurcations, particularly in the right and left pulmonary arteries of neonatal patients where vessel lengths are short and anatomy is highly constrained. The medium-angle geometry also can enable the transcatheter pulmonary flow reduction device 1000 to self-center during expansion, improving coaxial alignment with the pulmonary artery 120.

The device frame 1100 can be constructed in any appropriate manner. In selected embodiments, the device frame 1100 can be formed or otherwise manufactured from a metal material, a plastic material or any other device frame material suitable for use in cardiovascular implants. The device frame material preferably can be constructed from a biocompatible (or hemocompatible) metal that can provide sufficient radial strength, fatigue resistance and/or flexibility for delivery through small-diameter delivery catheter systems 2000 (shown in FIGS. 4A-D). Exemplary biocompatible metals can include, but are not limited to, a nickel-titanium alloy (or Nitinol), stainless steel (e.g., Alleima® 316LVM strip steel available from Alleima EMEA AB headquartered in Aktiebolag; Sweden, or equivalent medical grades), a cobalt-chromium alloy or any combination thereof. Nitinol may be selected for its superelasticity and shape-memory behavior, which facilitate self-expanding configurations, predictable radial recovery, and reliable deployment in tortuous neonatal vasculature. Stainless steel and cobalt-chromium alloys may be selected for their high modulus of elasticity, increased stiffness and enhanced radial force capability, which may be desirable in embodiments where the central waist region 1100W of the device frame 1100 must resist deformation during expansion and/or prolonged cyclic loading.

The device frame 1100, for example, can be laser-cut from a tubular stock of the device frame material. Additionally and/or alternatively, the device frame 1100, for example, can be laser-cut from rolled sheet stock subsequently formed into a tubular geometry and/or from composite structures including laminated or bonded layers of Nitinol and cobalt-chromium. Optional post-processing operations can include electropolishing, heat-setting (or shape-setting), surface passivation, and/or deposition of radiopaque (or polymeric) coatings, without limitation, to help enhance visibility, fatigue life and biocompatibility.

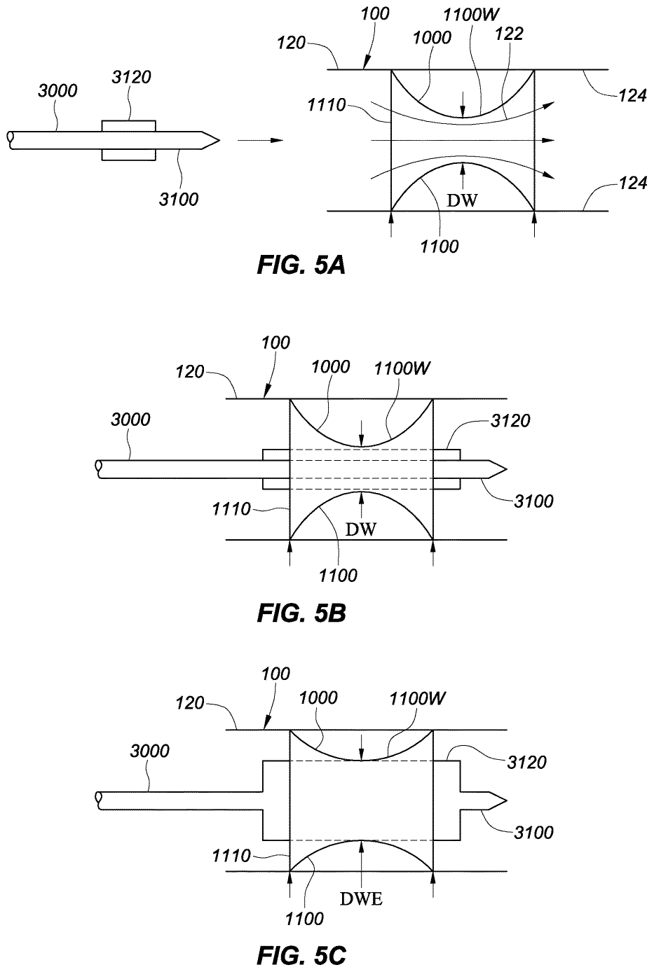

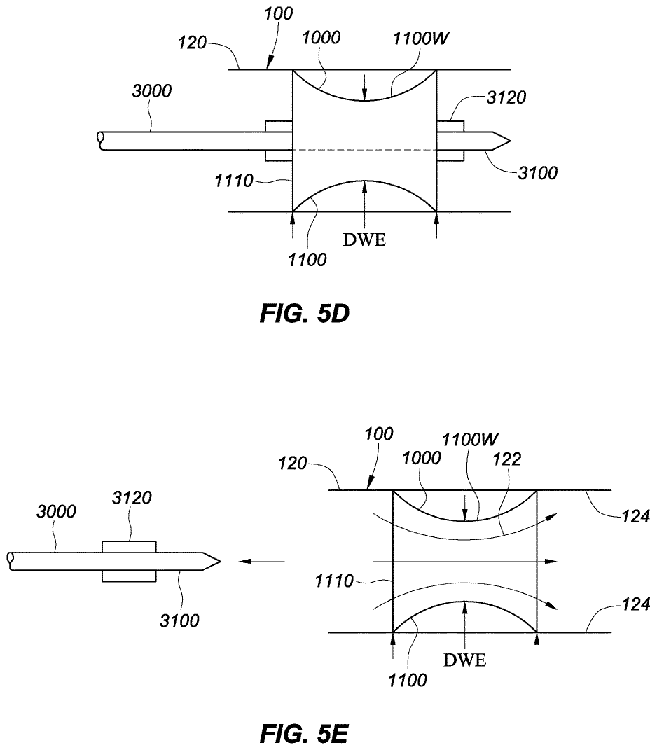

In selected embodiments, a hybrid frame construction may be used to balance self-expanding behavior with high radial strength in one or more predetermined localized regions of the device frame 1100. For example, the proximal and distal frame end regions 1100P, 1100D of the device frame 1100 can comprise one or more self-expanding Nitinol structures; whereas, the central waist region 1100W of the device frame 1100 can be manufactured from a balloon-expandable cobalt-chromium and/or stainless-steel. The proximal and distal frame end regions 1100P, 1100D thereby can be configured to self-expand for atraumatic anchoring and sealing; while, the central waist region 1100W can remain plastically deformable to permit controlled enlargement via a balloon (or other expansion) catheter system (or means) 3000 (shown in FIGS. 5A-E).

In selected embodiments, the distal strut end regions 1140D of the proximal and distal frame end regions 1100P, 1100D can help to anchor the device frame 1100 to the pulmonary artery 120. One or more of the distal strut end regions 1140D, for example, can have an end region width that is greater than a strut width of the device frame struts 1140 for enhancing the engagement between the device frame 1100 to the pulmonary artery 120. The engagement between the device frame 1100 to the pulmonary artery 120 optionally can be enhanced by providing the distal strut end regions 1140D of the proximal and distal frame end regions 1100P, 1100D as rigid distal end regions. The rigidity of the distal strut end regions 1140D can enable the distal strut end regions 1140D to serve as anchoring points without requiring additional retrieval or coupling features. Additionally and/or alternatively, the distal strut end regions 1140D can extend longitudinally from the proximal and distal frame end regions 1100P, 1100D for helping to prevent perforation of the pulmonary artery 120 and other patient vasculature and/or can extend radially outwardly from the proximal and distal frame end regions 1100P, 1100D for enhancing the engagement between the device frame 1100 to the pulmonary artery 120 when device frame 1100 is in an expanded state, such as a first stable expanded state or a second stable expanded state. The distal strut end regions 1140D thereby can provide mechanical engagement with the pulmonary artery 120, promoting axial stability and enhancing frictional retention under pulsatile blood flow 122.

The central waist region 1100W, additionally and/or alternatively, can incorporate a coil (not shown) formed from cobalt-chromium and/or stainless-steel. The coil can be positioned circumferentially around the external periphery 1120 of the central waist region 1100W and/or can function as a radially-expandable central waist region 1100W. The coil can support plastic expansion via the expansion catheter system 3000. Thereby, the coil can enable the internal dimension DW of the internal channel 1110 at the central waist region 1100W to be precisely adjusted while maintaining the self-expanding performance of the proximal and distal frame end regions 1100P, 1100D of the device frame 1100. The coil may be welded, soldered, mechanically interlocked and/or laser-bonded to the surrounding device frame 1100.

In selected embodiments, the entire device frame 1100 can be fabricated from cobalt-chromium and/or stainless steel. Such configurations may be desirable to increase radial strength at the proximal frame end region 1100P, the central waist region 1100W and/or the distal frame end region 1100D of the device frame 1100, particularly in patients 100 (shown in FIG. 1) who require a high anchoring force and/or in geometries in which robust resistance to recoil and/or deformation can be beneficial.

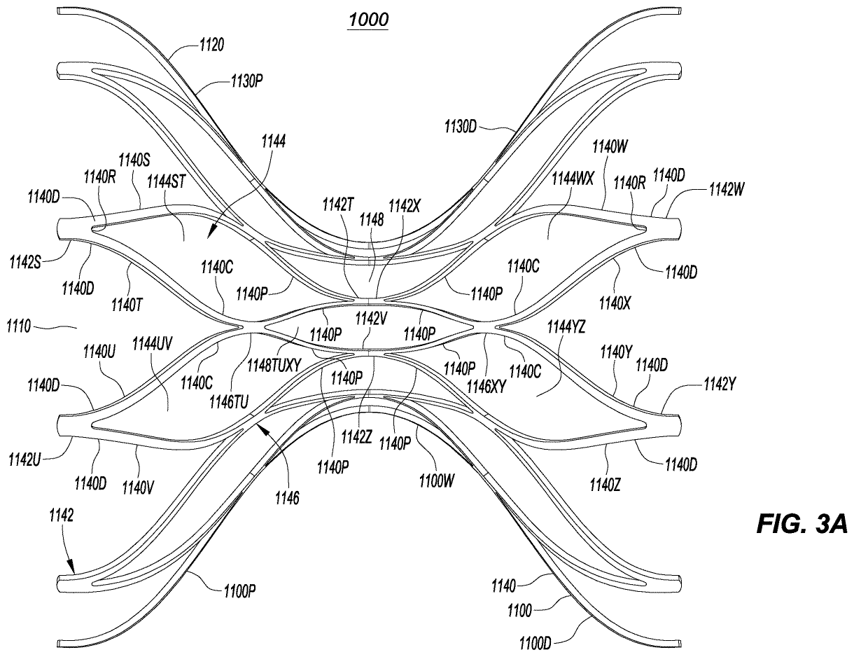

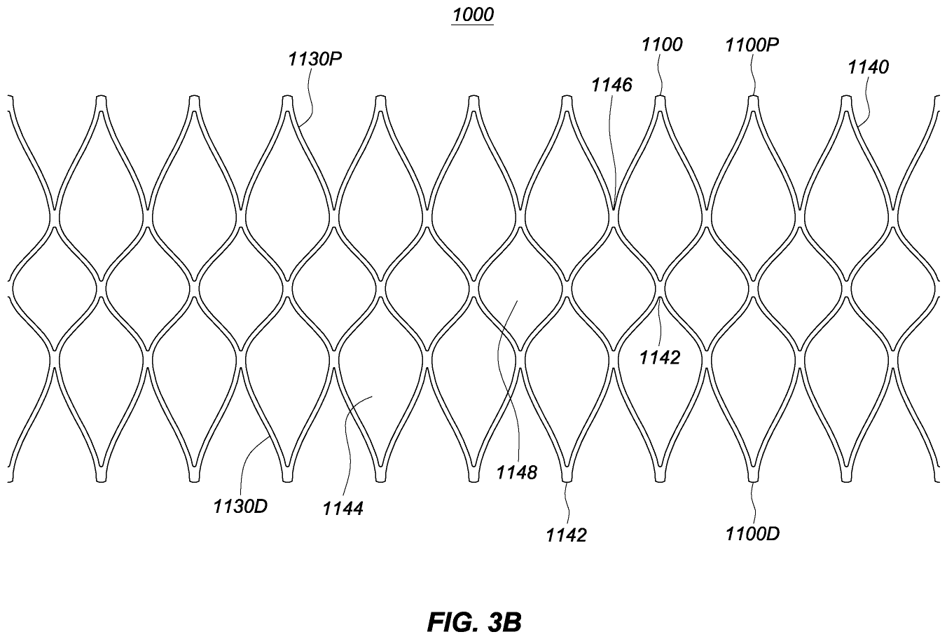

The device frame 1100 can be provided with any predetermined number of frame components being disposed in any suitable configuration. Turning to FIGS. 3A-B, for example, the device frame 1100 can include a proximal device frame lobe 1130P and a distal device frame lobe 1130D. The central waist region 1100W can be defined between the proximal and distal device frame lobes 1130P, 1130D. In selected embodiments, the proximal device frame lobe 1130P can comprise a first annular arrangement of device frame struts 1140 and can define a proximal portion of the internal channel 1110. The device frame struts 1140 of the first annular arrangement can be disposed in any suitable arrangement, such as a zigzag or other meandering arrangement.

For example, each of the device frame struts 1140 can include a proximal strut end region 1140P, a distal strut end region 1140D and a central region 1140C disposed between the proximal strut end region 1140P and the distal strut end region 1140D. The device frame struts 1140 can be grouped into pairs of adjacent device frame struts 1140, such as first paired device frame struts 1140S, 1140T and second paired device frame struts 1140U, 1140V in the manner illustrated in FIGS. 3A-B. The proximal strut end regions 1140P of each pair of adjacent device frame struts 1140 can intersect or otherwise cooperate to form a first frame end junction (or end joint) 1142, and the distal strut end regions 1140D of each pair of adjacent device frame struts 1140 can intersect or otherwise cooperate to form a second frame end junction 1142. In selected embodiments, the cooperation between two or more preselected device frame struts 1140 as set forth herein can include a coupling of the preselected device frame struts 1140 in any conventional manner.

As shown in FIG. 3A, the distal strut end region 1140D of the device frame strut 1140S can intersect or otherwise cooperate with the distal strut end region 1140D of the device frame strut 1140T to form a frame end junction 1142S; whereas, the proximal strut end region 1140P of the device frame strut 1140S can intersect or otherwise cooperate with the proximal strut end region 1140P of the device frame strut 1140T to form a frame end junction 1142T. Frame end junction 1142U can be formed at an intersection of the distal strut end regions 1140D of the device frame struts 1140U, 1140V, and/or frame end junction 1142V can be formed at an intersection of the proximal strut end regions 1140P of the device frame struts 1140U, 1140V.

Within the proximal device frame lobe 1130P, one or more growth frame cells 1144 can be defined by each pair of adjacent device frame struts 1140 in selected embodiments. The proximal device frame lobe 1130P can include any predetermined number of frame cells 1144 and is shown in FIGS. 3A-B as comprising ten frame cells 1144 for purposes of illustration only. In selected embodiments, the proximal device frame lobe 1130P can define two, four, six, eight, ten, twelve, fourteen, sixteen or more frame cells 1144, without limitation. By reducing the number of frame cells 1144 in each circumferential row defining the periphery 1120, the proximal device frame lobe 1130P can be formed or otherwise provided with device frame struts 1140 that have an increased strut thickness and/or an increased strut width while maintaining sufficient crimpability to be disposed on a low-profile or otherwise appropriately-sized delivery catheter system 2000 (shown in FIGS. 4A-D). The increased strut thickness and/or the increased strut width can help to enhance radial strength of the device frame 1100 and/or to provide improved vessel engagement upon expansion of the device frame 1100.

A selected pair of adjacent device frame struts 1140, for example, can intersect to form a frame cell 1144. Stated somewhat differently, the device frame struts 1140 can include meandering pairs of device frame struts 1140 that intersect at the frame end junctions 1142 and that define the one or more frame cells 1144 between the frame end junctions 1142. If the selected pair of adjacent device frame struts 1140 intersect twice, a closed frame cell 1144 can be formed between the adjacent device frame struts 1140. FIG. 3A, for instance, illustrates that a first frame cell 1144ST can formed between the cooperating first paired device frame struts 1140S, 1140T and that a second frame cell 1144UV can be formed between the cooperating second paired device frame struts 1140U, 1140V. Although shown in FIGS. 3A-B as having a generally diamond shape for purposes of illustration only, the frame cells 1144 of the proximal device frame lobe 1130P can have any suitable size, shape, diameter, cross-section or other dimension.

Additionally and/or alternatively, adjacent pairs of the adjacent device frame struts 1140 can intersect or otherwise cooperate at respective frame central junctions (or central joints) 1146. As shown in FIG. 3A, the device frame strut 1140T of the first paired device frame struts 1140S, 1140T can intersect or otherwise cooperate with the adjacent device frame strut 1140U of second paired device frame struts 1140U, 1140V. The central region 1140C of the device frame strut 1140T, for example, can intersect or otherwise cooperate with the central region 1140C of the adjacent device frame strut 1140U. The cooperating central regions 1140C of the device frame struts 1140T, 1140U thereby can form a frame central junction 1146TU.

The adjacent pairs of the adjacent device frame struts 1140, when cooperating, can provide the first annular arrangement of device frame struts 1140 with a first row of the frame cells 1144 disposed circumferentially around the external periphery 1120 of the proximal device frame lobe 1130P. Although shown and described as comprising one circumferential row of the frame cells 1144 for purposes of illustration only, the proximal device frame lobe 1130P can comprise any predetermined number of circumferential rows or other geometric configuration of the frame cells 1144, without limitation. A selection of the predetermined number of the frame cells 1144, the predetermined number of circumferential rows of the frame cells 1144 and the geometric arrangement (or configuration) of the frame cells 1144 advantageously can help to provide a balanced distribution of radial force, predictable crimping behavior, vessel conformity and/or uniform expansion of the proximal device frame lobe 1130P during deployment.

The distal device frame lobe 1130D can be provided in any suitable manner, including in a manner that is the same as, or different from, the manner by which the proximal device frame lobe 1130P is provided. In other words, the distal device frame lobe 1130D can comprise a second annular arrangement of device frame struts 1140 that is uniform with, or different from, the first annular arrangement of device frame struts 1140 of the proximal device frame lobe 1130P. The second annular arrangement of device frame struts 1140 of the distal device frame lobe 1130D can define a distal portion of the internal channel 1110 formed by the device frame 1100. The device frame struts 1140 of the second annular arrangement can be disposed in any suitable arrangement, such as a zigzag or other meandering arrangement.

In selected embodiments, each of the device frame struts 1140 can include a proximal strut end region 1140P, a distal strut end region 1140D and a central region 1140C disposed between the proximal strut end region 1140P and the distal strut end region 1140D in the manner discussed in more detail above. The device frame struts 1140 can be grouped into pairs of adjacent device frame struts 1140, such as first paired device frame struts 1140W, 1140X and second paired device frame struts 1140Y, 1140Z in the manner illustrated in FIG. 3A. The proximal strut end regions 1140P of each pair of adjacent device frame struts 1140 can intersect or otherwise cooperate to form a first frame end junction (or end joint) 1142, and the distal strut end regions 1140D of each pair of adjacent device frame struts 1140 can intersect or otherwise cooperate to form a second frame end junction 1142.

As shown in FIG. 3A, the distal strut end region 1140D of the device frame strut 1140W can intersect or otherwise cooperate with the distal strut end region 1140D of the device frame strut 1140X to form a frame end junction 1142W; whereas, the proximal strut end region 1140P of the device frame strut 1140W can intersect or otherwise cooperate with the proximal strut end region 1140P of the device frame strut 1140X to form a frame end junction 1142X. Frame end junction 1142Y can be formed at an intersection of the distal strut end regions 1140D of the device frame struts 1140Y, 1140Z, and/or frame end junction 1142Z can be formed at an intersection of the proximal strut end regions 1140P of the device frame struts 1140Y, 1140Z.

Within the distal device frame lobe 1130D, one or more frame cells 1144 can be defined by each pair of adjacent device frame struts 1140 in selected embodiments. The distal device frame lobe 1130D can include any predetermined number of frame cells 1144 and is shown in FIGS. 3A-B as comprising ten frame cells 1144 for purposes of illustration only. In selected embodiments, the distal device frame lobe 1130D can define four, six, eight, ten, twelve, fourteen or sixteen frame cells 1144, without limitation. By reducing the number of frame cells 1144 in each circumferential row defining the periphery 1120, the distal device frame lobe 1130D can be formed or otherwise provided with device frame struts 1140 that have an increased strut thickness and/or an increased strut width while maintaining sufficient crimpability to be disposed on a low-profile or otherwise appropriately-sized delivery catheter system 2000 (shown in FIGS. 4A-D). The increased strut thickness and/or the increased strut width can help to enhance radial strength of the device frame 1100 and/or to provide improved vessel engagement upon expansion of the device frame 1100.

A selected pair of adjacent device frame struts 1140, for example, can intersect to form a frame cell 1144. Stated somewhat differently, the device frame struts 1140 can include meandering pairs of device frame struts 1140 that intersect at the frame end junctions 1142 and that define one or more frame cells 1144 between the frame end junctions 1142. If the selected pair of adjacent device frame struts 1140 intersect twice, a closed frame cell 1144 can be formed between the adjacent device frame struts 1140. FIG. 3A, for instance, illustrates that a first frame cell 1144WX can formed between the cooperating first paired device frame struts 1140W, 1140X and that a second frame cell 1144YZ can be formed between the cooperating second paired device frame struts 1140Y, 1140Z. Although shown in FIGS. 3A-B as having a generally diamond shape for purposes of illustration only, the frame cells 1144 of the distal device frame lobe 1130D can have any suitable size, shape, diameter, cross-section or other dimension.

Additionally and/or alternatively, adjacent pairs of the adjacent device frame struts 1140 can intersect or otherwise cooperate at respective frame central junctions (or central joints) 1146. As shown in FIG. 3A, the device frame strut 1140X of the first paired device frame struts 1140W, 1140X can intersect or otherwise cooperate with the adjacent device frame strut 1140Y of second paired device frame struts 1140Y, 1140Z. The central region 1140C of the device frame strut 1140X, for example, can intersect or otherwise cooperate with the central region 1140C of the adjacent device frame strut 1140Y. The cooperating central regions 1140C of the device frame struts 1140X, 1140Y thereby can form a frame central junction 1146XY.

The adjacent pairs of the adjacent device frame struts 1140, when cooperating, can provide the second annular arrangement of device frame struts 1140 with a first row of the frame cells 1144 disposed circumferentially around the external periphery 1120 of the distal device frame lobe 1130D. Although shown and described as comprising one row of the frame cells 1144 for purposes of illustration only, the distal device frame lobe 1130D can comprise any predetermined number of circumferential rows or other geometric configuration of the frame cells 1144, without limitation. A selection of the predetermined number of the frame cells 1144, the predetermined number of circumferential rows of the frame cells 1144 and the geometric arrangement (or configuration) of the frame cells 1144 advantageously can help to provide a balanced distribution of radial force, predictable crimping behavior, vessel conformity and/or uniform expansion of the distal device frame lobe 1130D during deployment.

The proximal device frame lobe 1130P and the distal device frame lobe 1130D can cooperate to form the device frame 1100. As illustrated in FIGS. 3A-B, the proximal strut end regions 1140P of the first annular arrangement of device frame struts 1140 that comprise the proximal device frame lobe 1130P can be configured to intersect or otherwise cooperate with the proximal strut end regions 1140P of the second annular arrangement of device frame struts 1140 that comprise the distal device frame lobe 1130D. The frame end junction 1142T formed at the intersection of the proximal strut end regions 1140P of the device frame struts 1140S, 1140T of the proximal device frame lobe 1130P, for example, can be configured to cooperate with the frame end junction 1142X formed at the intersection of the proximal strut end regions 1140P of the device frame struts 1140W, 1140X of the distal device frame lobe 1130D. Additionally and/or alternatively, the frame end junction 1142V formed at the intersection of the proximal strut end regions 1140P of the device frame struts 1140U, 1140V of the proximal device frame lobe 1130P can be configured to cooperate with the frame end junction 1142Z formed at the intersection of the proximal strut end regions 1140P of the device frame struts 1140Y, 1140Z of the distal device frame lobe 1130D.

The central waist region 1100W of the device frame 1100 can be defined or otherwise provided at the intersection between the proximal device frame lobe 1130P and the distal device frame lobe 1130D. The intersection between the proximal strut end regions 1140P of the first annular arrangement of device frame struts 1140 that comprise the proximal device frame lobe 1130P and the proximal strut end regions 1140P of the second annular arrangement of device frame struts 1140 that comprise the distal device frame lobe 1130D advantageously can define a plurality of re-expandable growth frame cells 1148 of the central waist region 1100W. Frame cell 1148TUXY, for example, is shown as being formed between the cooperating proximal strut end regions 1140P of the device frame struts 1140T, 1140X and the cooperating proximal strut end regions 1140P of the device frame struts 1140U, 1140Y. The device frame 1100 can include any predetermined number of frame cells 1148 that can be disposed circumferentially around the external periphery 1120 of the central waist region 1100W of the device frame 1100 and is shown in FIGS. 3A-B as comprising ten frame cells 1148 for purposes of illustration only.

The frame cells 1148 advantageously can enable the internal dimension DW (shown in FIG. 2) of the internal channel 1110 of the central waist region 1100W to be controllable or otherwise adjustable. A size, shape, diameter, cross-section or other dimension of the frame cells 1148, for example, can be increased for increasing the internal dimension DW of the internal channel 1110 and/or can be decreased for decreasing the internal dimension DW of the internal channel 1110. Although shown in FIGS. 3A-B as having a generally diamond shape for purposes of illustration only, the frame cells 1148 can have any suitable size, shape, diameter, cross-section or other dimension. Additionally and/or alternatively, the device frame 1100 can comprise any predetermined number of circumferential rows or other geometric configuration of the frame cells 1148 disposed longitudinally along an axial length of the device frame 1100, without limitation.

Selection of the predetermined number of the frame cells 1144, the predetermined number of the frame cells 1148, the predetermined number of circumferential rows of the frame cells 1144 and frame cells 1148 and/or the geometric arrangement (or configuration) of the frame cells 1144 and frame cells 1148 advantageously can help to provide a balanced distribution of radial force, predictable crimping behavior, vessel conformity and/or uniform expansion of the device frame 1100 during implantation and/or deployment. An exemplary suitable geometric configuration of the frame cells 1144 and frame cells 1148 can include three circumferential rows disposed longitudinally along the axial length of the device frame 1100 and with each row having ten frame cells 1144 or frame cells 1148 as illustrated by the device frame 1100 of FIGS. 3A-B.

Although shown and described as comprising separate first and second annular arrangement of device frame struts 1140 for purposes of illustration only, the device frame 1100 can be formed in any suitable manner. The device frame 1100, in selected embodiments, can comprise a single annular arrangement of device frame struts 1140, wherein one or more of the frame struts 1140 extend from the proximal frame end region 1100P of the device frame 1100 to the distal frame end region 1100D of the device frame 1100. For example, the device frame strut 1140S and the device frame strut 1140W can be provided as a first composite device frame strut 1140; whereas, the device frame strut 1140T and the device frame strut 1140X can be provided as a second composite device frame strut 1140. Additionally and/or alternatively, the device frame strut 1140U and the device frame strut 1140Y can be provided as a third composite device frame strut 1140, and/or the device frame strut 1140V and the device frame strut 1140Z can be provided as a fourth composite device frame strut 1140. Each of the composite device frame struts 1140 can extend from the proximal frame end region 1100P to the distal frame end region 1100D of the device frame 1100 and/or can be provided in any suitable arrangement, such as a zigzag or other meandering arrangement. In selected embodiments, the device frame 1100 can be provided as a scaffold or other mesh-like device frame.

In use, the transcatheter pulmonary flow reduction device 1000 can be configured for implantation in the patent 100 as illustrated in FIGS. 4A-D. The transcatheter pulmonary flow reduction device 1000, for example, can be provided for implantation within the pulmonary artery 120, such as a selected branch pulmonary artery 120A, 120B (shown in FIG. 28) or other lumen, of the patient 100 for percutaneously reducing (or partially restricting) the pulmonary blood flow 122 within the pulmonary artery 120. Turning FIG. 4A, the device frame 1100 can be crimped or otherwise provided in the implantation state with the implantation dimension DI and coupled with, or otherwise disposed on, a catheter distal end region 2100 of a delivery catheter system 2000. The transcatheter pulmonary flow reduction device 1000, for example, can be tethered to the delivery catheter system 2000 via suture (not shown). In selected embodiments, the delivery catheter system 2000 can have an inner diameter of at least one millimeter, and/or the device frame 1100 can be configured to be disposed within the delivery catheter system 2000. The catheter distal end region 2100 of the delivery catheter system 2000 can be advanced through the vasculature (not shown) of the patent 100 and into the heart 110 until the device frame 1100 is positioned at a predetermined location within the pulmonary artery 120 as shown in FIG. 4B.

Once positioned at the predetermined location within the pulmonary artery 120, the device frame 1100 can be implanted and otherwise deployed in the patient 100. The device frame 1100, for example, can be radially expanded from the implantation state to a first stable expanded state. In the first stable expanded state, the external periphery 1120 of the device frame 1100 can engage the pulmonary artery 120 for maintaining the transcatheter pulmonary flow reduction device 1000 at the predetermined location. As illustrated in FIG. 4C, for example, the proximal frame end region 1100P of the device frame 1100 can be expanded to the predetermined dimension DP for engaging a first region of an internal lumen surface 124 within the pulmonary artery 120, and/or the distal frame end region 1100D of the device frame 1100 can be expanded to the predetermined dimension DD for engaging a second region of the internal lumen surface 124. In selected embodiments, the external periphery 1120 of the device frame 1100 can have an hourglass shape in the first stable expanded state.

The engagement with the internal lumen surface 124 of the pulmonary artery 120 can help maintain the device frame 1100 at the predetermined location within the pulmonary artery 120. Stated somewhat differently, the proximal and distal frame end regions 1100P, 1100D can help to anchor the device frame 1100 within the pulmonary artery 120. The internal channel 1110 of the device frame 1100 likewise can be radially expanded for permitting the pulmonary blood flow 122 (shown in FIG. 4A) within the pulmonary artery 120 and can be defined with the internal dimension DW at the waist region 1100W of the device frame 1100 for reducing the pulmonary blood flow 122 within the pulmonary artery 120.

The transcatheter pulmonary flow reduction device 1000 can be expanded to first stable expanded state in any suitable manner. In selected embodiments, the delivery catheter system 2000 can include an implant expansion system (or means) (not shown), such as a balloon, for expanding the device frame 1100 from the implantation state to the first stable expanded state. The implant expansion system, for example, can be disposed at the catheter distal end region 2100 of the delivery catheter system 2000 and can be received within the internal channel 1110 of the device frame 1100 when the transcatheter pulmonary flow reduction device 1000 is coupled with the delivery catheter system 2000. Once the device frame 1100 is positioned at the predetermined location within the pulmonary artery 120, the implant expansion system can expand to expand the device frame 1100 from the implantation state to the first stable expanded state. The proximal and distal frame end regions 1100P, 1100D of the device frame 1100 in the first stable expanded state can engage the internal lumen surface 124 within the pulmonary artery 120, and/or the internal channel 1110 of the device frame 1100 can be radially expanded to define the internal dimension DW at the waist region 1100W for reducing the pulmonary blood flow 122 within the pulmonary artery 120 in the manner set forth in more detail above.

Additionally and/or alternatively, the transcatheter pulmonary flow reduction device 1000 can comprise a self-expanding device. The device frame 1100, for example, can be formed or otherwise constructed from a self-expanded material in the manner discussed in more detail above. Once positioned at the predetermined location within the pulmonary artery 120, the device frame 1100 can self-expand from the implantation state to the first stable expanded state. The proximal and distal frame end regions 1100P, 1100D of the device frame 1100 in the first stable expanded state can engage the internal lumen surface 124 within the pulmonary artery 120, and/or the internal channel 1110 of the device frame 1100 can be radially expanded to define the internal dimension DW at the waist region 1100W for reducing the pulmonary blood flow 122 within the pulmonary artery 120 in the manner set forth in more detail above.

After implantation and deployment of the transcatheter pulmonary flow reduction device 1000 is complete, the delivery catheter system 2000 can be withdrawn from the patient 100 as shown in FIG. 4D. The proximal and distal frame end regions 1100P, 1100D of the device frame 1100 in the first stable expanded state can continue to engage the internal lumen surface 124 within the pulmonary artery 120 for maintaining the transcatheter pulmonary flow reduction device 1000 at the predetermined location within the pulmonary artery 120. The pulmonary blood flow 122 likewise can pass through the pulmonary artery 120 via the internal channel 1110 of the device frame 1100. The internal dimension DW of the internal channel 1110 at the waist region 1100W thereby can continue to reduce the pulmonary blood flow 122 within the pulmonary artery 120. Although shown and described with reference to FIGS. 4A-D as being implanted and deployed via a delivery catheter system 2000 for purposes of illustration only, the transcatheter pulmonary flow reduction device 1000 can implanted and/or deployed via any suitable medical procedure, including surgical implantation and/or deployment, without limitation.

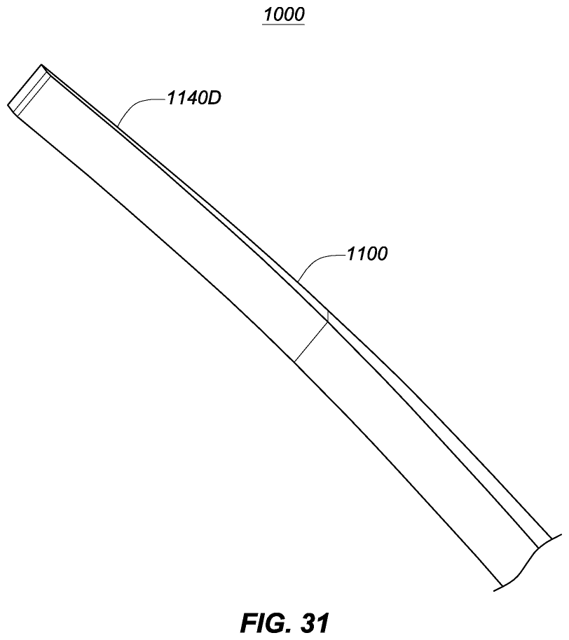

In various embodiments, the distal strut end regions 1140D of the proximal frame end region 1100P and/or the distal frame end region 1100D of the transcatheter pulmonary flow reduction device 1000 may be shape-set in predetermined geometric configurations to tailor a degree and mechanism of engagement and retention when deployed within the pulmonary artery 120 (shown in FIG. 1). The predetermined geometric configurations can be uniform and/or different among the distal strut end regions 1140D of the transcatheter pulmonary flow reduction device 1000. Turning to FIG. 31, for example, the distal strut end regions 1140D can be shape-set substantially parallel to the vessel wall of the pulmonary artery 120 (shown in FIG. 1) when the transcatheter pulmonary flow reduction device 1000 is disposed in one of the stable expanded states. This configuration of the transcatheter pulmonary flow reduction device 1000 advantageously can help to limit aggressive penetration or focal contact with the vessel wall while still providing sufficient frictional engagement to promote axial stability and retention.

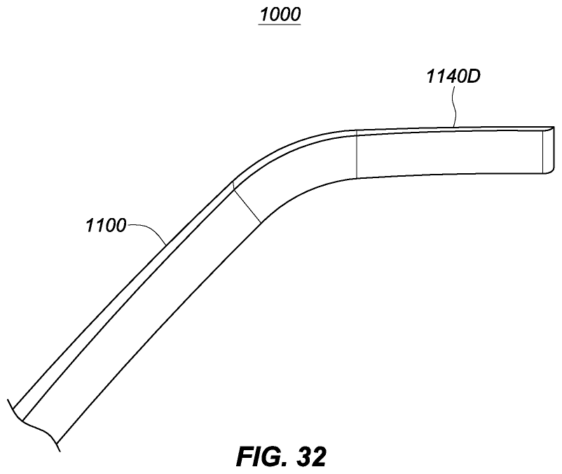

One or more of the distal strut end regions 1140D of the transcatheter pulmonary flow reduction device 1000 optionally can be shape-set to flare radially outwardly in a substantially straight orientation relative to the longitudinal axis of the transcatheter pulmonary flow reduction device 1000 in the manner shown in FIG. 32. This outwardly-flaring geometry can increase direct engagement between the distal strut end regions 1140D of the transcatheter pulmonary flow reduction device 1000 and the vessel wall of the pulmonary artery 120 (shown in FIG. 1), thereby enhancing anchoring and resistance to migration under pulsatile flow conditions.

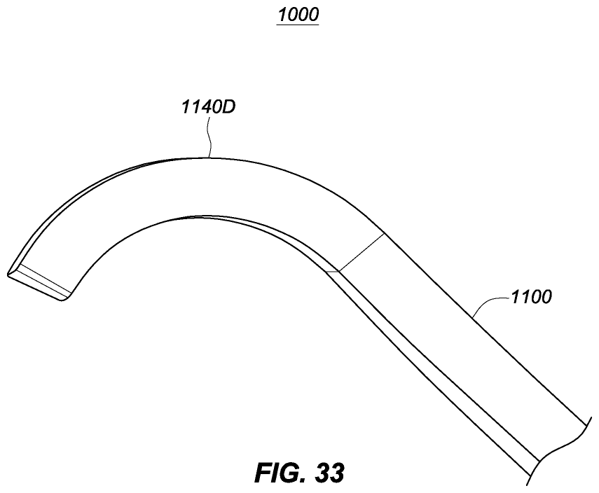

Additionally and/or alternatively, the distal strut end regions 1140D of the transcatheter pulmonary flow reduction device 1000 can be shape-set to curve radially outwardly toward the vessel wall as illustrated in FIG. 33. The radially outwardly geometry of the distal strut end regions 1140D advantageously can effectively increase the dimension DP of the proximal frame end region 1100P and/or the dimension DD of the distal frame end region 1100D. This curvature of the distal strut end regions 1140D, in other words, can produce broad, direct contact with the vessel wall of the pulmonary artery 120 (shown in FIG. 1) and can provide enhanced mechanical retention through increased surface contact and radial engagement.

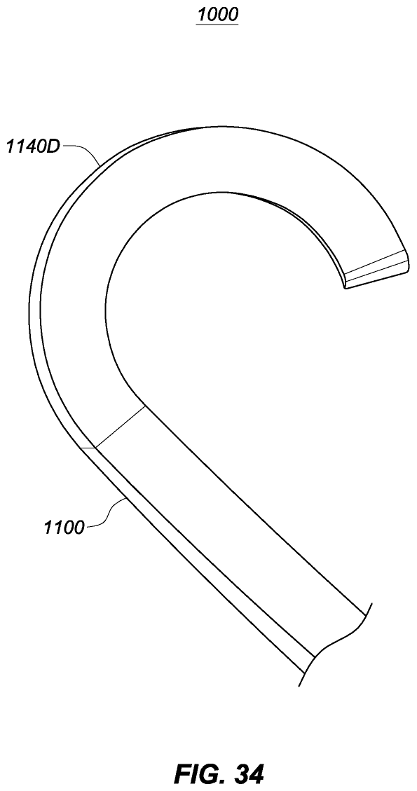

FIG. 34 shows yet another exemplary geometric configuration of the distal strut end regions 1140D, wherein the distal strut end regions 1140D of the transcatheter pulmonary flow reduction device 1000 can be shape-set to curve away from the vessel wall of the pulmonary artery 120 (shown in FIG. 1). This configuration advantageously can help to substantially reduce and/or eliminate direct contact between the distal strut end regions 1140D of the transcatheter pulmonary flow reduction device 1000 and the vessel wall, causing retention to rely primarily on a radial strength and outward expansion of the proximal frame end region 1100P and/or the distal frame end region 1100D rather than focal apical engagement. Such embodiments may be advantageous in reducing vessel irritation, minimizing tissue ingrowth, and improving chronic biocompatibility while maintaining adequate positional stability

The transcatheter pulmonary flow reduction device 1000 advantageously can be configured for reducing the amount of the pulmonary blood flow 122 within the pulmonary artery 120 in a controllable or otherwise adjustable manner. An amount of flow restriction provided by the transcatheter pulmonary flow reduction device 1000, for example, can be adjusted after implantation (or deployment) of the transcatheter pulmonary flow reduction device 1000 based up on one or more hemodynamic needs of the patient 100. Turning to FIGS. 5A-E, a balloon (or other expansion) catheter system 3000 can be introduced into the patient 100 for adjusting the internal dimension DW of the internal channel 1110 of the device frame 1100. The expansion catheter system 3000, for example, can be introduced for adjusting the internal dimension DW of the internal channel 1110 during the implantation of the transcatheter pulmonary flow reduction device 1000 and/or at a time in a separate medical procedure after the implantation of the transcatheter pulmonary flow reduction device 1000.

Turning to FIG. 5A, the implanted (or deployed) device frame 1100 is shown as being disposed the first stable expanded state. The implanted (or deployed) device frame 1100 can be engaged with the internal lumen surface 124 within the pulmonary artery 120, and the internal channel 1110 of the device frame 1100 can be defined with the internal dimension DW at the waist region 1100W in the manner discussed in more detail above with reference to the transcatheter pulmonary flow reduction device 1000 of FIGS. 4A-D. The internal dimension DW of the internal channel 1110 can reduce the pulmonary blood flow 122 within the pulmonary artery 120.

The expansion catheter system 3000 can include a distal end region 3100 with an implant expansion system 3120, such as a balloon. The implant expansion system 3120 can be provided with an initial unexpanded state and can be activated to expand to an expanded state. With the implant expansion system 3120 in the unexpanded state, the distal end region 3100 of the expansion catheter system 3000 can be advanced through the vasculature (not shown) of the patent 100 and into the heart 110 until the implant expansion system 3120 is at least partially disposed within the internal channel 1110 of the deployed device frame 1100 as shown in FIG. 5B.

Once disposed within the internal channel 1110 of the device frame 1100, the implant expansion system 3120 can be activated for expanding from the unexpanded state and to the first expanded state. The waist region 1100W of the device frame 1100, for example, can define the internal channel 1110 with internal dimension DW when the device frame 1100 is in the first stable expanded state. Advantageously, the implant expansion system 3120 advantageously can be configured for radially expanding the device frame 1100 from the first stable expanded state to a second stable expanded state. The implant expansion system 3120, in other words, can be activated for radially re-expanding the internal channel 1110 at the waist region 1100W from the internal dimension DW to a second (or enlarged) internal dimension DWE that is greater than the internal dimension DW as illustrated in FIG. 5C.

In the second stable expanded state, the external periphery 1120 of the device frame 1100 can continue to engage the pulmonary artery 120 for maintaining the transcatheter pulmonary flow reduction device 1000 at the predetermined location. The activated implant expansion system 3120 optionally can be configured to further expand the proximal and distal frame end regions 1100P, 1100D of the device frame 1100, as needed, to maintain the engagement between the external periphery 1120 of the device frame 1100 and the internal lumen surface 124 within the pulmonary artery 120. In selected embodiments, no further expansion of the proximal and distal frame end regions 1100P, 1100D of the device frame 1100 may be required for the transcatheter pulmonary flow reduction device 1000 to reduce the amount of blood flow 112 from the pulmonary artery 120 to the lungs of the patient in the controllable or otherwise adjustable manner. The implant expansion system 3120, in other words, can be activated for radially re-expanding the internal channel 1110 at the waist region 1100W from the internal dimension DW to the second internal dimension DWE without adjusting the external periphery 1120 of the device frame 1100. In selected embodiments, the external periphery 1120 of the device frame 1100 can maintain the hourglass shape in the second stable expanded state.

After the internal channel 1110 at the waist region 1100W has been expanded to the second internal dimension DWE, the implant expansion system 3120 can be deactivated. The implant expansion system 3120 thereby can contract from the expanded state back to the unexpanded state as shown in FIG. 5D. FIG. 5E shows that the expansion catheter system 3000 then can be withdrawn from the patient 100. The proximal and distal frame end regions 1100P, 1100D of the device frame 1100 in the second stable expanded state can continue to engage the internal lumen surface 124 within the pulmonary artery 120 for maintaining the transcatheter pulmonary flow reduction device 1000 at the predetermined location within the pulmonary artery 120.

The pulmonary blood flow 122 likewise can continue to pass through the pulmonary artery 120 via the internal channel 1110 of the device frame 1100. The second internal dimension DWE of the internal channel 1110 at the waist region 1100W can reduce the pulmonary blood flow 122 within the pulmonary artery 120 with the pulmonary blood flow 122 permitted by the internal channel 1110 with the second internal dimension DWE being greater than the pulmonary blood flow 122 permitted by the internal channel 1110 with the internal dimension DW. In other words, the transcatheter pulmonary flow reduction device 1000 with the second internal dimension DWE can reduce the pulmonary blood flow 122 through the pulmonary artery 120, but the reduction in the pulmonary blood flow 122 is less than the reduction in the pulmonary blood flow 122 provided by the transcatheter pulmonary flow reduction device 1000 with the internal dimension DW. Although shown and described with reference to FIGS. 5A-E and being adjusted after implantation via the expansion catheter system 3000 for purposes of illustration only, the transcatheter pulmonary flow reduction device 1000 can be expanded via subsequent introduction of any suitable catheter system (or means) or other medical device, including a delivery catheter system 2000 (shown in FIGS. 4A-D), into the patient 100 (shown in FIG. 1).

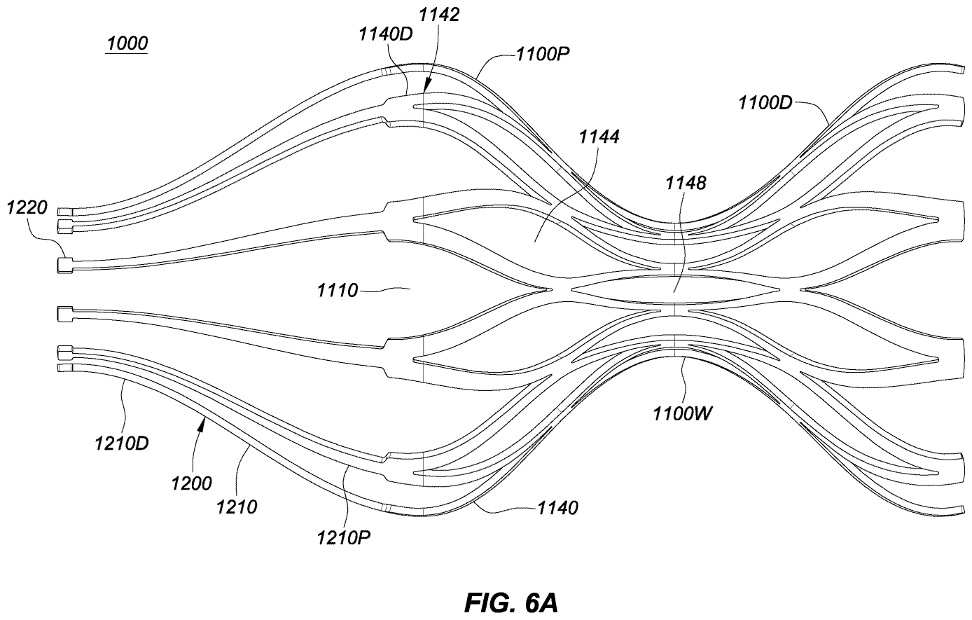

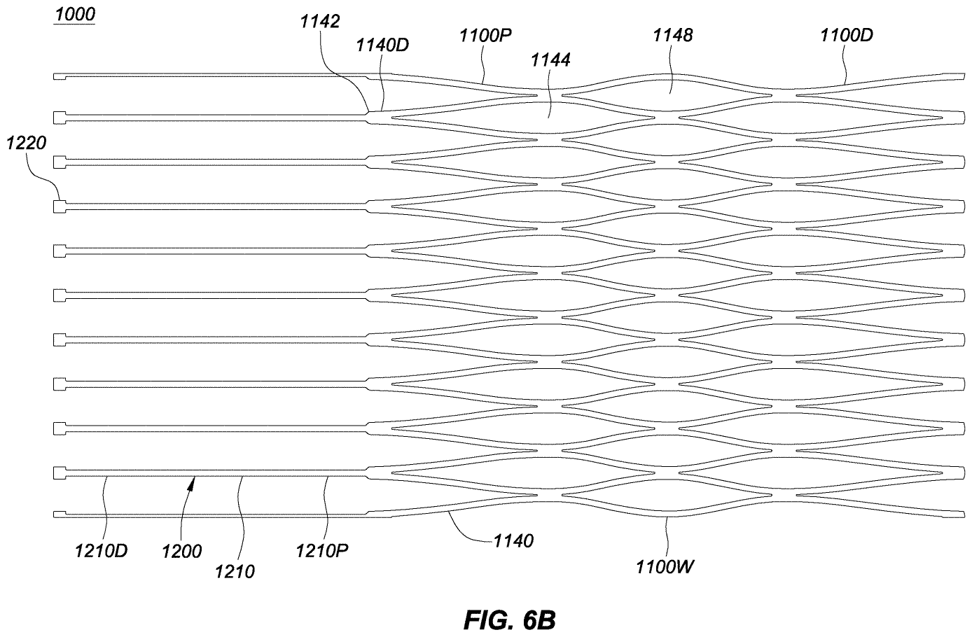

In the manner discussed above, the transcatheter pulmonary flow reduction device 1000 optionally can comprise a removable transcatheter pulmonary flow reduction device that can be deployed in, and later retrieved from, the pulmonary artery 120. The transcatheter pulmonary flow reduction device 1000 of FIGS. 6A-B, for example, can include an hourglass-shaped device frame 1100 with a central waist region 1100W disposed between proximal and distal frame end regions 1100P, 1100D and defining an internal channel 1110 in the manner discussed in more detail above with reference to the device frame 1100 shown in FIGS. 2 and 3A-B and is shown as further comprising an optional device retrieval system (or means) 1200. The device retrieval system 1200 advantageously can facilitate recapture, repositioning, retrieval and/or removal of the transcatheter pulmonary flow reduction device 1000 after deployment. In selected embodiments, the delivery catheter system 2000 (shown in FIGS. 4A-D), the expansion catheter system 3000 (shown in FIGS. 5A-E) or other implant retrieval system can be utilized to engage the device retrieval system 1200 and to recapture, reposition, retrieve and/or remove the transcatheter pulmonary flow reduction device 1000 within the pulmonary artery 120 (shown in FIG. 1). The implant retrieval system, in other words, can include the delivery catheter system 2000, the expansion catheter system 3000 or any other suitable catheter system or other medical device.

The device retrieval system 1200 advantageously can enable one-sided recapture, repositioning, retrieval and/or removal of the transcatheter pulmonary flow reduction device 1000. Stated somewhat differently, the transcatheter pulmonary flow reduction device 1000 can be recaptured, repositioned, retrieved and/or removed by engaging only the proximal frame end region 1100P of the device frame 1100 via the device retrieval system 1200. An ability to recapture, reposition, retrieve and/or remove the transcatheter pulmonary flow reduction device 1000 can help to eliminate a need for repeated surgical procedures and/or can allow for temporary and/or staged management of pulmonary flow. Although shown and described herein as extending from the proximal frame end region 1100P of the device frame 1100 for purposes of illustration only, the device retrieval system 1200 optionally can extend from the distal frame end region 1100D of the device frame 1100. Stated somewhat differently, the device retrieval system 1200 can be coupled or otherwise integrated with the proximal frame end region 1100P and/or the distal frame end region 1100D of the device frame 1100.

The device retrieval system 1200 can be provided in any suitable matter. As shown in FIG. 6A-B, for example, the device retrieval system 1200 can comprise one or more device retrieval members 1210. In selected embodiments, the device retrieval members 1210 can comprise shallow-angled, unrestrained tendrils (or struts) that are formed from a flexible material and with a slender size, shape, width, depth, cross-section or other dimension. The device retrieval members 1210, in other words, can extend parallel with the external periphery 1120 of the device frame 1100 and/or can be biased or otherwise extend radially inwardly toward a longitudinal axis of the internal channel 1110 defined by the device frame 1100. The device retrieval members 1210 can extend radially inwardly toward the longitudinal axis of the internal channel 1110 at any suitable angle, such as any predetermined angle within a first angle range between zero degrees and seventy degrees, or within any angle subrange of the first angle range, without limitation.

Turning to FIG. 6A, the device retrieval members 1210 are illustrated as extending proximally from respective distal strut end regions 1140D (or frame end junctions 1142) of the proximal frame end region 1100P of the device frame 1100. Each device retrieval member 1210 can comprise an elongate member with proximal and distal retrieval member end regions 1210P, 1210D. The proximal retrieval member end regions 1210P of the device retrieval members 1210 can be coupled or otherwise cooperate with the respective distal strut end regions 1140D (or frame end junctions 1142) of the proximal frame end regions 1100P of the device frame 1100. The distal retrieval member end regions 1210D of the device retrieval members 1210 can extend proximally from the proximal frame end region 1100P of the device frame 1100.

Upon release from the delivery catheter system 2000, the proximally-directed device retrieval members 1210 can naturally flare radially outwardly to help enhance an engagement for snaring the device frame 1100 during a recapturing, repositioning, retrieval and/or removal procedure, while still maintaining a compact, atraumatic profile. In selected embodiments, the proximally-directed device retrieval members 1210 can converge toward a longitudinal axis of the internal channel 1110 defined by the device frame 1100 and/or can be secured, bonded or otherwise retained. The device retrieval members 1210, for example, can be mechanically retained via a dedicated coupling (or retention) device (not shown). The retention device can be affixed over, or otherwise disposed at, the distal retrieval member end regions 1210D and can be configured for interfacing with the catheter distal end region 2100 (shown in FIGS. 4A-D) of the delivery catheter system 2000 for allowing controlled coupling, decoupling, repositioning and/or chronic retrieval of the transcatheter pulmonary flow reduction device 1000. A geometry of the device retrieval members 1210 advantageously can provide enhanced clinical accessibility for balloon-based adjustment and/or improved compatibility with mechanical coupling systems.

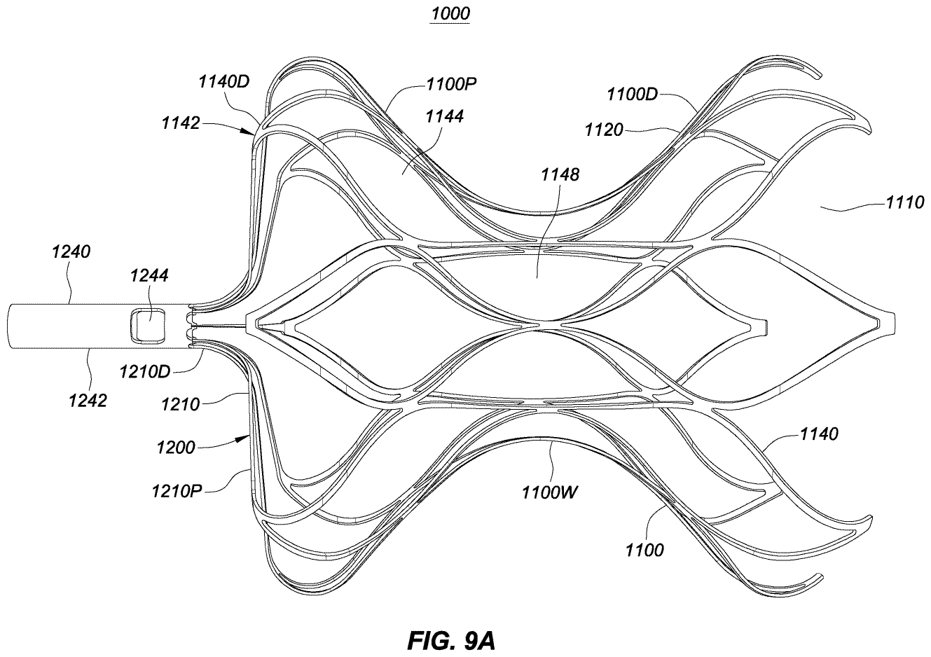

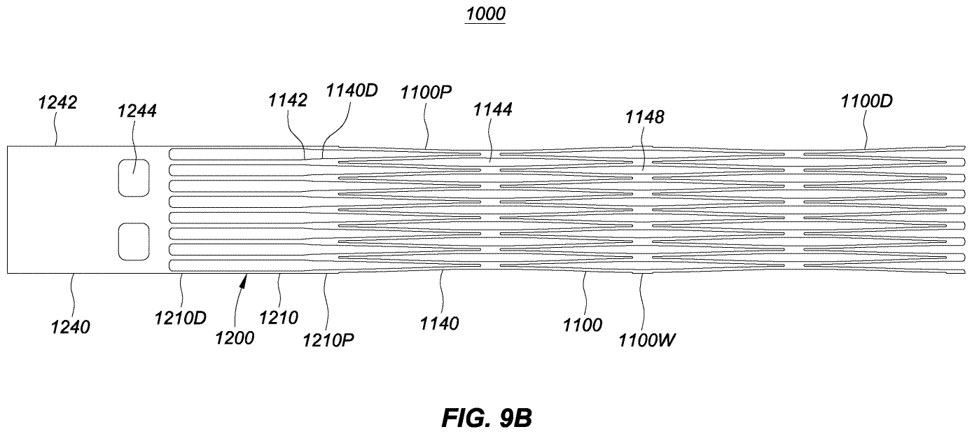

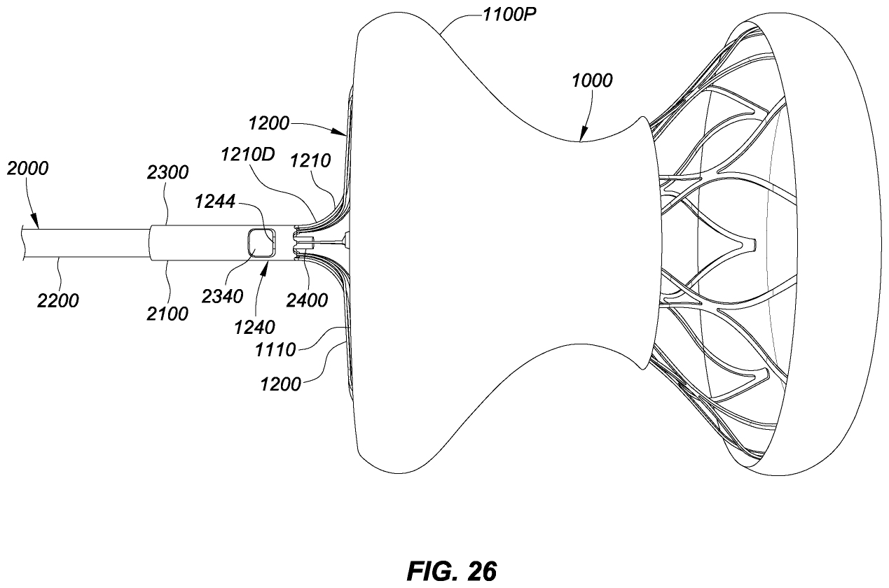

In selected embodiments, one or more of the distal retrieval member end regions 1210D of the device retrieval members 1210 can include a device engagement (or anchoring) system 1220. In certain embodiments, the distal retrieval member end regions 1210D of the device retrieval members 1210 may converge toward a longitudinal axis of the internal channel 1110 defined by the device frame 1100 and/or may be secured, bonded, or otherwise mechanically retained using a dedicated coupling (or retention) mechanism (not shown). This coupling mechanism may be affixed over the distal retrieval member end regions 1210D of the device retrieval members 1210as illustrated in FIG. 26 and/or the distal retrieval member end regions 1210D of the device retrieval members 1210 may converge to a hollow pin/coupler or other coupling device (or member or means) 1240 (shown in FIG. 9A) and configured to interface with the catheter distal end region 2100 (shown in FIGS. 4A-D) of the delivery catheter system 2000 (shown in FIGS. 4A-D) for allowing controlled coupling, decoupling, repositioning, and/or chronic retrieval of the transcatheter pulmonary flow reduction device 1000. The device engagement system 1220 advantageously can be configured for enhancing an engagement between at least one of the device retrieval members 1210 and the implant retrieval system. Stated somewhat differently, the device engagement system 1220 can help to provide a secure engagement between the device frame 1100 and the implant retrieval system. The device engagement system 1220, in selected embodiments, can help to increase capture reliability of the transcatheter pulmonary flow reduction device 1000 under fluoroscopic visualization and/or to facilitate recapture of the transcatheter pulmonary flow reduction device 1000 during both acute and chronic retrieval scenarios.

Exemplary implant retrieval systems can include, but are not limited to, gooseneck snares, suture-loop snares and/or wire-loop-based retrieval systems. The device engagement system 1220 advantageously can be provided with any predetermined geometry (or geometric profile) that is suitable for engaging (and enhancing the engagement with) with one or more preselected implant retrieval system (or means). In selected embodiments, the device engagement systems 1220 can be uniform, and/or different, among the device retrieval members 1210. Stated somewhat differently, the device engagement systems 1220 can be configured with different geometries to optimize snare engagement, fluoroscopic visibility and/or recapture reliability. The device engagement systems 1220, for example, can comprise a footed region and/or a ledged surface that is sized for enhancing a secure engagement by the preselected implant retrieval system.

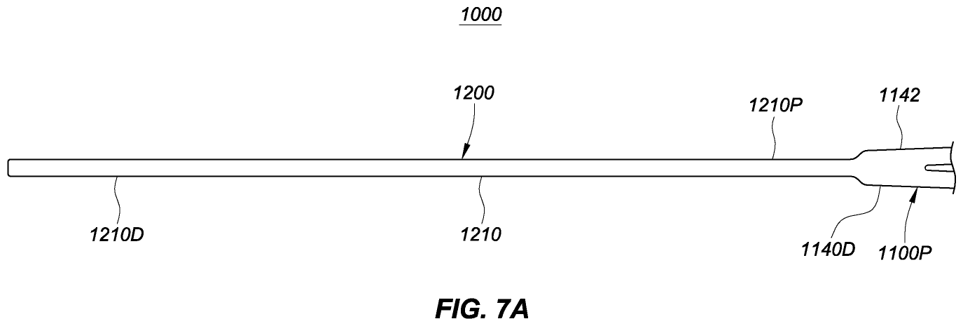

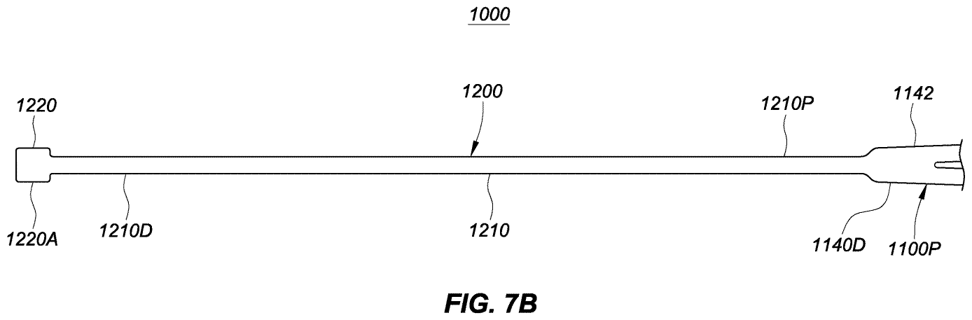

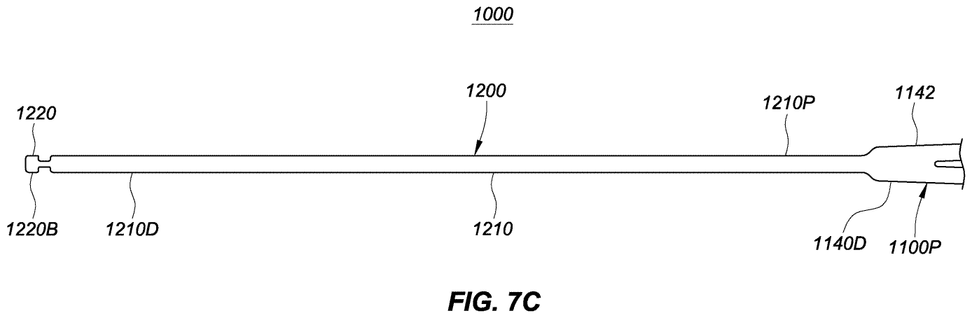

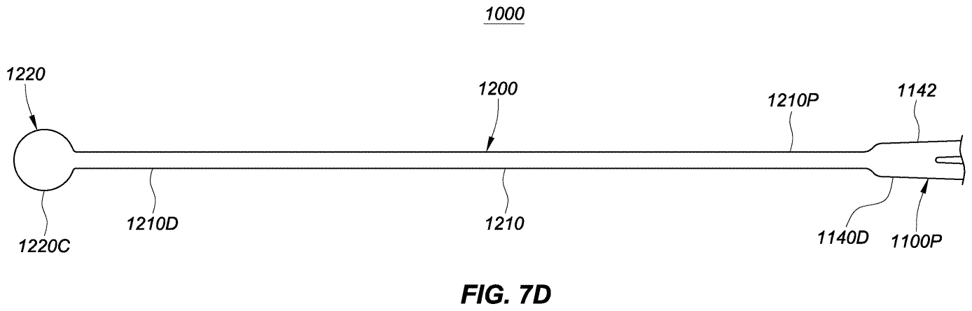

The device engagement systems 1220 can be uniform and/or different among the distal retrieval member end regions 1210D. Turning to FIG. 7A, for example, the transcatheter pulmonary flow reduction device 1000 is shown as including a basic device retrieval member 1210. The basic device retrieval member 1210 of FIG. 7A is not associated with a device engagement system 1220 as shown in FIGS. 6A-B. In contrast, the device retrieval members 1210 of FIGS. 7B-D are shown as including respective device engagement systems 1220. As illustrated in FIG. 7B, for example, the device retrieval member 1210 can include a first device engagement system (or means) 1220A that has a substantially quadrilateral (or square) profile with radiused corners for preventing vessel trauma while providing a broad capture surface for enhancing a secure engagement by the preselected implant retrieval system, such as a gooseneck snare and/or wire-loop retrieval device.

FIG. 7C shows another embodiment of the device retrieval member 1210 that can include a second device engagement system (or means) 1220B with a T-shaped profile with rounded corners, which provides lateral protrusions that resist slippage during snare engagement. Additionally and/or alternatively, the device retrieval member 1210 of FIG. 7D is shown as including a third device engagement system 1220C with a rounded, circular profile for providing a smooth, atraumatic capture interface. Other alternative embodiments of the device engagement system 1220 can include, but are not limited to, a device engagement system 1220 with a fully-radiused tip incorporating one or more lateral retention notches sized to mechanically engage a snare loop and/or a device engagement system 1220 with a simplified square tip with radiused edges for balancing atraumatic profile, manufacturability and predictable capture performance. The geometric profiles of the device engagement systems 1220, for example, can be selected based on desired retrieval characteristics, manufacturing constraints and/or preferred interaction with the preselected implant retrieval system.

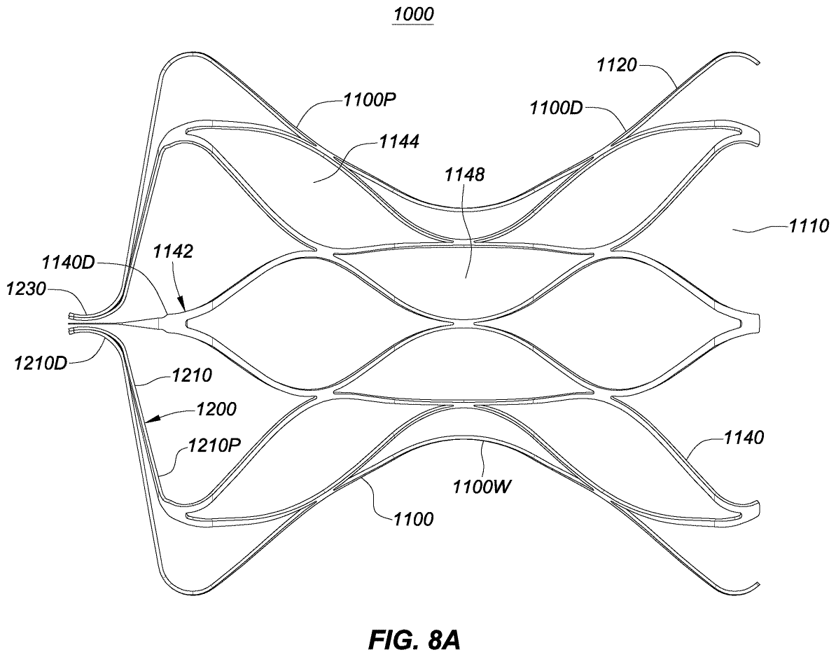

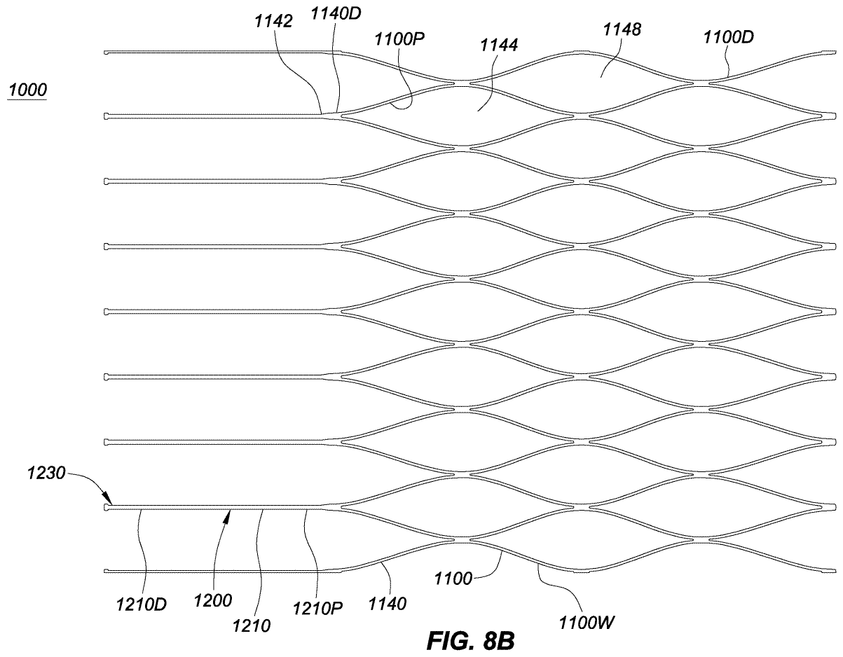

An exemplary alternative embodiment of the transcatheter pulmonary flow reduction device 1000 is illustrated in FIGS. 8A-B. Turning to FIGS. 8A-B, the transcatheter pulmonary flow reduction device 1000 is shown as comprising an hourglass-shaped device frame 1100 with a central waist region 1100W disposed between proximal and distal frame end regions 1100P, 1100D and defining an internal channel 1110 in the manner discussed in more detail above with reference to the device frame 1100 shown in FIGS. 2, 3A-B and 6A-B. The external periphery 1120 of the proximal frame end region 1100P and the external periphery 1120 of the central waist region 1100W can define a first frame taper angle; whereas, the external periphery 1120 of the distal frame end region 1100F and the external periphery 1120 of the central waist region 1100W can define a second frame taper angle. The first and second frame taper angles can comprise any suitable angle, such as any predetermined angle within a taper angle range between twenty degrees and eighty degrees, or within any angle subrange of the taper angle range, without limitation. Although preferably comprising uniform angles, the first and second frame taper angles can be different in selected embodiments and/or in selected applications.

The transcatheter pulmonary flow reduction device 1000 can comprise a device frame 1100 with any suitable geometric pattern or other arrangement of the frame cells 1144, 1148. As shown in FIGS. 8A-B, for example, the device frame 1100 can comprise an eight-cell circumferential pattern with three longitudinal rows of diamond-shaped frame cells 1144, 1148. This geometric pattern of the frame cells 1144, 1148 advantageously can allow for thicker and/or wider strut geometries of the device frame struts 1140 while still maintaining sufficient crimpability for loading into a low-profile or otherwise appropriately-sized delivery catheter system 2000 (shown in FIGS. 4A-D). The increased strut thickness can enhance radial strength and/or can provide improved vessel engagement upon expansion. Additionally and/or alternatively, the distal strut end regions 1140D of the device frame 1100 can be curved or otherwise positioned away from the vessel wall or internal lumen surface 124 (shown in FIG. 5A) of the pulmonary artery 120 (shown in FIG. 1) for reducing risk of vessel trauma and/or to minimize a likelihood of tissue ingrowth. The distal strut end regions 1140D, in other words, can be biased or otherwise extend radially inwardly toward a longitudinal axis of the internal channel 1110 defined by the device frame 1100. The thicker and/or wider strut geometries of the device frame struts 1140 of the device frame struts 1140 and/or the positioning of the distal strut end regions 1140D away from the vessel wall 124 can help to promote radial engagement between the device frame 1100 and the vessel wall 124 through broad strut-to-wall contact rather than discrete apical contact.

The device retrieval system 1200 of FIGS. 8A-B generally can be provided in the manner discussed in more detail above with reference to the device retrieval system 1200 of FIGS. 6A-B but with the one or more device retrieval members 1210 being biased or otherwise extending radially inwardly toward a longitudinal axis of the internal channel 1110 defined by the device frame 1100. In the manner discussed above with reference to the tendrils of FIGS. 6A-B, for example, at least one of the device retrieval members 1210 of the device retrieval system 1200 of FIGS. 8A-B can be provided as tendrils (or struts) that can be formed from a flexible material and that have a slender size, shape, width, depth, cross-section or other dimension.

The device retrieval members 1210 of the device retrieval system 1200 can comprise deep-angled, unrestrained tendrils that can merge or otherwise converge to form a common capture point (or member or system or means) 1230. The device retrieval members 1210 can extend radially inwardly toward the longitudinal axis of the internal channel 1110 at any suitable angle, such as any predetermined angle within a second angle range between forty degrees and eighty degrees, or within any angle subrange of the second angle range, without limitation. The deep-angle of the device retrieval members 1210 relative to the longitudinal axis of the internal channel 1110 advantageously can help increase a spacing between adjacent device retrieval members 1210, thereby improving access to the central waist region 1100W of the device frame 1100 for post-implant expansion or other intraluminal adjustment procedures for the transcatheter pulmonary flow reduction device 1000. Additionally and/or alternatively, the deep-angle of the device retrieval members 1210 can reduce an axial length of the device retrieval system 1200. The reduced axial length of the device retrieval system 1200 advantageously can help to minimize an overall footprint of the transcatheter pulmonary flow reduction device 1000 within the pulmonary artery 120 (shown in FIG. 1) and/or to reduce a risk of obstructing or otherwise jailing branch vessels that are adjacent to the pulmonary artery 120.

The delivery catheter system 2000 (shown in FIGS. 4A-D), the expansion catheter system 3000 (shown in FIGS. 5A-E) or other implant retrieval system can be utilized to engage the common capture member 1230 and to recapture, reposition, retrieve and/or remove the transcatheter pulmonary flow reduction device 1000 within the pulmonary artery 120 (shown in FIG. 1). Thereby, the device retrieval system 1200 advantageously can enable one-sided recapture, repositioning, retrieval and/or removal of the transcatheter pulmonary flow reduction device 1000. The transcatheter pulmonary flow reduction device 1000, in other words, can be recaptured, repositioned, retrieved and/or removed by engaging only the proximal frame end region 1100P of the device frame 1100 via the device retrieval system 1200. An ability to recapture, reposition, retrieve and/or remove the transcatheter pulmonary flow reduction device 1000 can help to eliminate a need for repeated surgical procedures and/or can allow for temporary and/or staged management of pulmonary flow.

FIGS. 9A-B illustrate another exemplary alternative embodiment of the transcatheter pulmonary flow reduction device 1000. Turning to FIGS. 9A-B, the transcatheter pulmonary flow reduction device 1000 is shown as comprising an hourglass-shaped device frame 1100 with a central waist region 1100W disposed between proximal and distal frame end regions 1100P, 1100D and defining an internal channel 1110 in the manner discussed in more detail above with reference to the device frame 1100 shown in FIGS. 2, 3A-B and 6A-B. The external periphery 1120 of the proximal frame end region 1100P and the external periphery 1120 of the central waist region 1100W can define a first frame taper angle; whereas, the external periphery 1120 of the distal frame end region 1100F and the external periphery 1120 of the central waist region 1100W can define a second frame taper angle. The first and second frame taper angles can comprise any suitable angle, such as any predetermined angle within a taper angle range between twenty degrees and eighty degrees, or within any angle subrange of the taper angle range, without limitation. Although preferably comprising uniform angles, the first and second frame taper angles can be different in selected embodiments and/or in selected applications.

The transcatheter pulmonary flow reduction device 1000 can comprise a device frame 1100 with any suitable geometric pattern or other arrangement of the frame cells 1144, 1148. As shown in FIGS. 9A-B, for example, the device frame 1100 can comprise an eight-cell circumferential pattern with three longitudinal rows of diamond-shaped frame cells 1144, 1148. This geometric pattern of the frame cells 1144, 1148 advantageously can allow for thicker and/or wider strut geometries of the device frame struts 1140 while still maintaining sufficient crimpability for loading into a low-profile or otherwise appropriately-sized delivery catheter system 2000 (shown in FIGS. 4A-D). The increased strut thickness can enhance radial strength and/or can provide improved vessel engagement upon expansion.

Additionally and/or alternatively, the distal strut end regions 1140D of the device frame 1100 can be curved or otherwise positioned away from the vessel wall or internal lumen surface 124 (shown in FIG. 5A) of the pulmonary artery 120 (shown in FIG. 1) for reducing risk of vessel trauma and/or to minimize a likelihood of tissue ingrowth. The distal strut end regions 1140D, in other words, can be biased or otherwise extend radially inwardly toward a longitudinal axis of the internal channel 1110 defined by the device frame 1100. The thicker and/or wider strut geometries of the device frame struts 1140 of the device frame struts 1140 and/or the positioning of the distal strut end regions 1140D away from the vessel wall 124 can help to promote radial engagement between the device frame 1100 and the vessel wall 124 through broad strut-to-wall contact rather than discrete apical contact.

The device retrieval system 1200 of FIGS. 9A-B generally can be provided in the manner discussed in more detail above with reference to the device retrieval system 1200 of FIGS. 8A-B with the one or more device retrieval members 1210 being biased or otherwise extending radially inwardly toward a longitudinal axis of the internal channel 1110 defined by the device frame 1100. In the manner discussed above with reference to the tendrils of FIGS. 8A-B, for example, at least one of the device retrieval members 1210 of the device retrieval system 1200 of FIGS. 9A-B can be provided as tendrils (or struts) that can be formed from a flexible material and that have a slender size, shape, width, depth, cross-section or other dimension.

The device retrieval members 1210 can extend radially inwardly toward the longitudinal axis of the internal channel 1110 at any suitable angle, such as any predetermined angle between forty-five degrees and eighty degrees, without limitation. The deep-angle of the device retrieval members 1210 advantageously can help increase a spacing between adjacent device retrieval members 1210, thereby improving access to the central waist region 1100W of the device frame 1100 for post-implant expansion or other intraluminal adjustment procedures for the transcatheter pulmonary flow reduction device 1000. Additionally and/or alternatively, the deep-angle of the device retrieval members 1210 can reduce an axial length of the device retrieval system 1200. The reduced axial length of the device retrieval system 1200 advantageously can help to minimize an overall footprint of the transcatheter pulmonary flow reduction device 1000 within the pulmonary artery 120 (shown in FIG. 1) and/or to reduce a risk of obstructing or otherwise jailing branch vessels that are adjacent to the pulmonary artery 120.

As shown in FIGS. 9A-B, the device retrieval members 1210 of the device retrieval system 1200 can comprise deep-angled, unrestrained tendrils that can merge or otherwise converge at a coupling device (or member or means) 1240 for engaging the delivery catheter system 2000 (shown in FIGS. 4A-D), the expansion catheter system 3000 (shown in FIGS. 5A-E) or other implant retrieval system (or means) can be utilized to engage the device retrieval system 1200 for recapturing, repositioning, retrieving and/or removing the transcatheter pulmonary flow reduction device 1000 within the pulmonary artery 120 (shown in FIG. 1). The coupling device 1240, in selected embodiments, can be provided as a hollow pin as illustrated in FIG. 9A. Stated somewhat differently, the coupling device 1240 can comprise a coupler member housing 1242 for defining a periphery of the coupling device 1240. Advantageously, the coupling device 1240 can provide a dedicated coupling interface for recapture, repositioning, retrieval and/or removal of the transcatheter pulmonary flow reduction device 1000.

The coupling device 1240, as shown in FIGS. 9A-B, optionally can define one or more engagement (or retention) slots, windows or other openings 1244 for enhancing an engagement with the delivery catheter system 2000, the expansion catheter system 3000 or other implant retrieval system. The delivery catheter system 2000, the expansion catheter system 3000 or other implant retrieval system can be utilized to engage the coupling device 1240 and to recapture, reposition, retrieve and/or remove the transcatheter pulmonary flow reduction device 1000 within the pulmonary artery 120. Each of the engagement openings 1244 can have a predetermined geometric profile. Exemplary geometric profiles of the engagement openings 1244 can include, but are not limited to, a rectangular profile and/or a square profile. The engagement openings 1244 can be provided in any suitable manner, such as via laser-cutting opposing sides of the coupler member housing 1242.

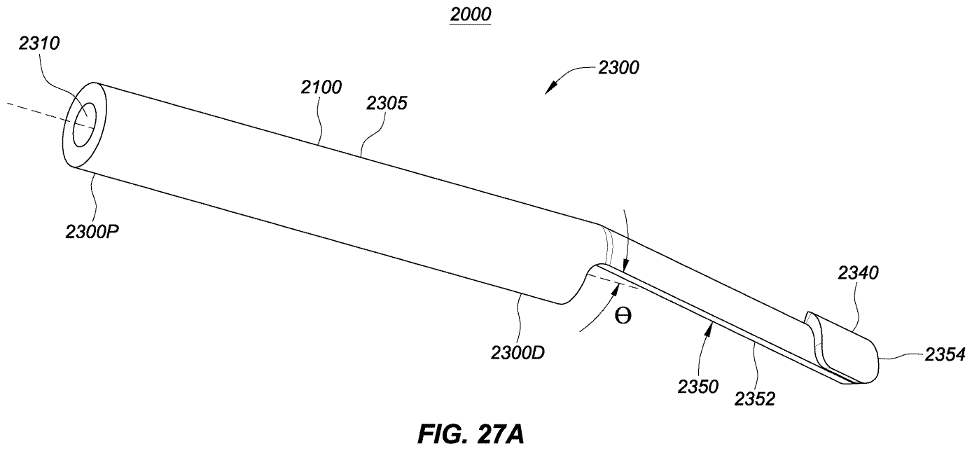

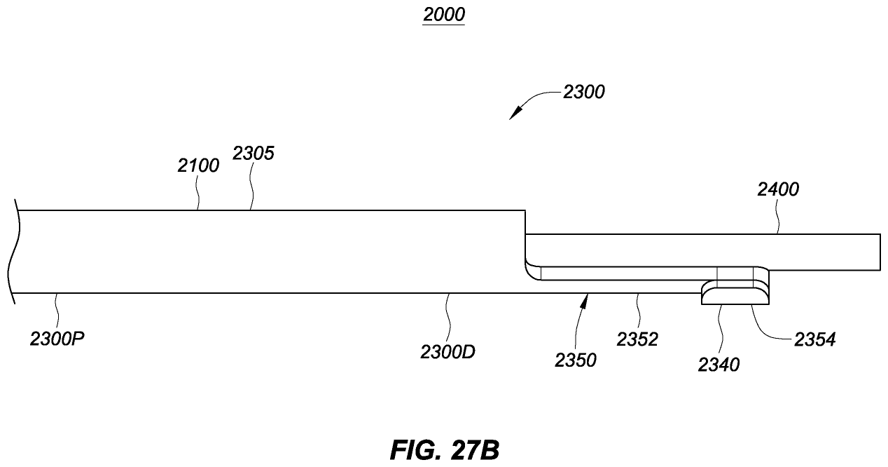

In selected embodiments, the engagement openings 1244 can be dimensioned for mechanically interfacing with a complementary interface engagement device (or member) 2340 (shown in FIGS. 26 and 27A-B) disposed, for example, at the catheter distal end region 2100 (shown in FIGS. 4A-D) of the delivery catheter system 2000 (shown in FIGS. 4A-D), the expansion catheter system 3000 (shown in FIGS. 5A-E) or other implant retrieval system. The catheter distal end region 2100 (shown in FIGS. 4A-D) of the implant retrieval system, for example, can clip, lock, expand or otherwise engage the engagement openings 1244 to initiate and/or maintain a (temporary) mechanical connection with the transcatheter pulmonary flow reduction device 1000 during advancement, positioning, partial deployment, repositioning, retrieval, recapture and/or removal of the transcatheter pulmonary flow reduction device 1000. After the transcatheter pulmonary flow reduction device 1000 has been positioned and the desired degree of pulmonary flow reduction is confirmed, the coupling between the transcatheter pulmonary flow reduction device 1000 and the implant retrieval system can be terminated, allowing controlled decoupling between the transcatheter pulmonary flow reduction device 1000 and the implant retrieval system.

The engagement openings 1244 optionally can facilitate retrieval and/or recapture of the transcatheter pulmonary flow reduction device 1000. The engagement openings 1244 defined by the coupler member housing 1242, for example, can allow the implant retrieval system, such as a snare (not shown), a gooseneck snare (not shown), a suture-loop snare (not shown) and/or a wire-loop capture device(not shown), to securely engage the coupling device 1240 for acute repositioning and/or chronic removal of the transcatheter pulmonary flow reduction device 1000 from the pulmonary artery 120. The coupling device 1240 advantageously can enable reliable engagement between the coupling device 1240 and the implant retrieval system from multiple approach angles and/or can reduce dependency on tendril-tip capture alone. The coupling device 1240 thereby can provide a robust dual-purpose interface for coupling during deployment and/or retrieval during follow-up procedures.

The device retrieval system 1200 thereby can enable one-sided recapture, repositioning, retrieval and/or removal of the transcatheter pulmonary flow reduction device 1000. The transcatheter pulmonary flow reduction device 1000, in other words, can be recaptured, repositioned, retrieved and/or removed by engaging only the proximal frame end region 1100P of the device frame 1100 via the device retrieval system 1200. An ability to recapture, reposition, retrieve and/or remove the transcatheter pulmonary flow reduction device 1000 can help to eliminate a need for repeated surgical procedures and/or can allow for temporary and/or staged management of pulmonary flow.

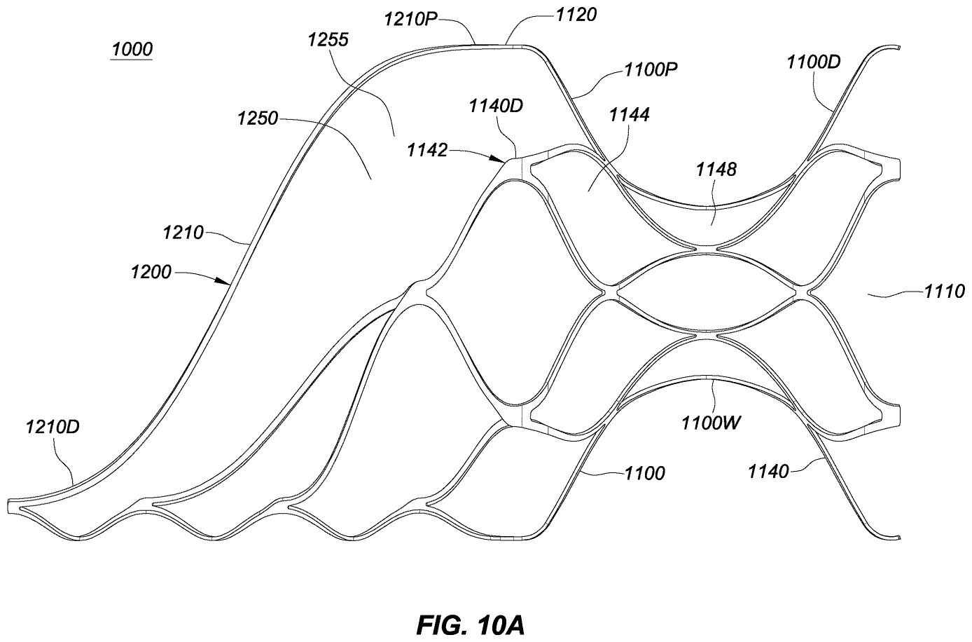

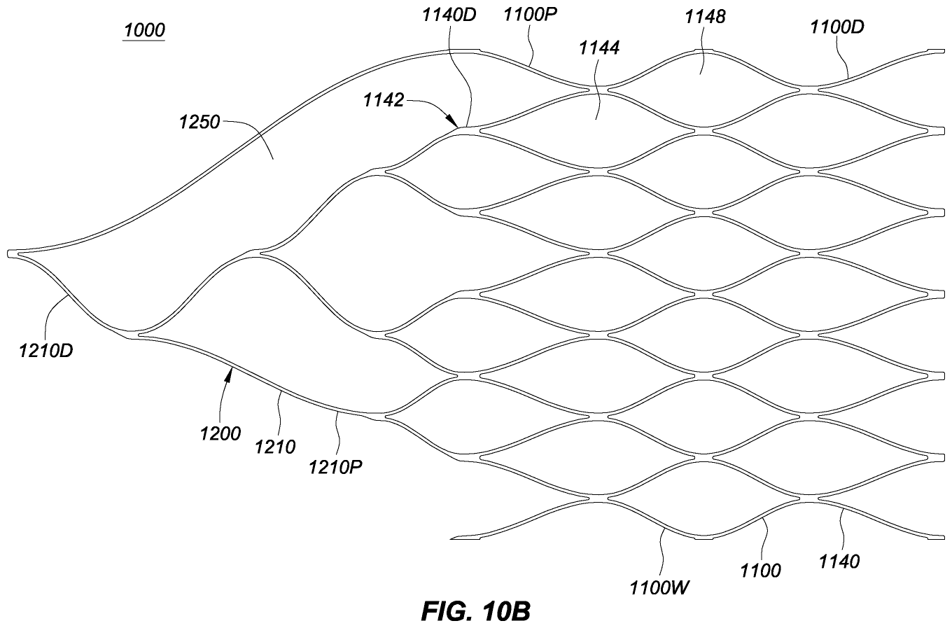

Turning to FIGS. 10A-B, yet another exemplary alternative embodiment of the transcatheter pulmonary flow reduction device 1000 is illustrated. The transcatheter pulmonary flow reduction device 1000 is shown as comprising an hourglass-shaped device frame 1100 with a central waist region 1100W disposed between proximal and distal frame end regions 1100P, 1100D and defining an internal channel 1110 in the manner discussed in more detail above with reference to the device frame 1100 shown in FIGS. 2, 3A-B and 6A-B. The external periphery 1120 of the proximal frame end region 1100P and the external periphery 1120 of the central waist region 1100W can define a first frame taper angle; whereas, the external periphery 1120 of the distal frame end region 1100F and the external periphery 1120 of the central waist region 1100W can define a second frame taper angle. The first and second frame taper angles can comprise any suitable angle, such as any predetermined angle within a taper angle range between twenty degrees and eighty degrees, or within any angle subrange of the taper angle range, without limitation. Although preferably comprising uniform angles, the first and second frame taper angles can be different in selected embodiments and/or in selected applications.

The device frame 1100 of FIGS. 10A-B, for example, can comprise a six-cell circumferential pattern with three longitudinal rows of diamond-shaped frame cells 1144, 1148. Relative to device frames 1100 with circumferential patterns comprising eight or more frame cells 1144, 1148, the geometric pattern of frame cells 1144, 1148 shown in FIGS. 10A-B advantageously can allow for still thicker and/or wider strut geometries of the device frame struts 1140 while still maintaining sufficient crimpability for loading into a low-profile or otherwise appropriately-sized delivery catheter system 2000 (shown in FIGS. 4A-D). For example, the device frame 1100 can be compatible for loading into a low-profile delivery catheter system 2000 suitable for neonatal and other pediatric applications. The thicker strut construction advantageously can provide enhanced radial engagement with the vessel wall or internal lumen surface 124 (shown in FIG. 5A) of the pulmonary artery 120 (shown in FIG. 1) and improved resistance to deformation under pulsatile loading. The further-increased strut thickness optionally can enhance radial strength and/or can provide improved vessel engagement upon expansion.

Additionally and/or alternatively, the distal strut end regions 1140D of the device frame 1100 can be curved or otherwise positioned away from the vessel wall or internal lumen surface 124 (shown in FIG. 5A) of the pulmonary artery 120 (shown in FIG. 1) for reducing risk of vessel trauma and/or to minimize a likelihood of tissue ingrowth. The distal strut end regions 1140D, in other words, can be biased or otherwise extend radially inwardly toward a longitudinal axis of the internal channel 1110 defined by the device frame 1100. The thicker and/or wider strut geometries of the device frame struts 1140 of the device frame struts 1140 and/or the positioning of the distal strut end regions 1140D away from the vessel wall 124 can help to promote radial engagement between the device frame 1100 and the vessel wall 124 through broad strut-to-wall contact rather than discrete apical contact.

FIGS. 10A-B likewise illustrate another exemplary alternative embodiment of the device retrieval system 1200. The device retrieval system 1200 can be provided in the manner discussed in more detail above with reference to the device retrieval system 1200 of FIGS. 6A-B but with the device retrieval members 1210 defining at least one enlarged retrieval system cell 1250. The proximally-directed device retrieval members 1210, in selected embodiments, can include proximal retrieval member end regions 1210P that extend radially outwardly from the device frame 1100 and distal retrieval member end regions 1210D that can converge. A single enlarged, diamond-shaped cell 1250 thereby can be defined between adjacent device retrieval members 1210 and/or positioned along the external periphery 1120 of the proximal frame end region 1100P. In the manner discussed above with reference to the tendrils of FIGS. 6A-B, for example, at least one of the device retrieval members 1210 of the device retrieval system 1200 of FIGS. 10A-B can be provided as tendrils (or struts) that can be formed from a flexible material and that have a slender size, shape, width, depth, cross-section or other dimension.

As shown in FIGS. 10A-B, the distal strut end regions 1140D can extend radially outwardly from the device frame 1100. The circumferential rows of frame cells 1144 can converge into a series of one or more larger diamond-shaped retrieval system cells 1250. The retrieval system cells 1250 can transition proximally and ultimately merge into a single enlarged diamond-shaped retrieval system cell 1255 located on an outer region of the proximal frame end region 1100P of the device frame 1100. The merging of the retrieval system cells 1250 into the single enlarged diamond-shaped retrieval system cell 1255 advantageously can help to eliminate a need for separate device retrieval members 1210 extending into the internal channel 1110 defined by the transcatheter pulmonary flow reduction device 1000. In other words, the converging-cell architecture can provide a structurally-robust proximal retrieval interface located at the external periphery 1120 of the transcatheter pulmonary flow reduction device 1000 rather than within the internal channel 1110, thereby maintaining an open internal channel 1110 and/or improving easier access to the central waist region 1100W of the device frame 1100 for balloon-based expansion or other catheter-based interventions.

The delivery catheter system 2000 (shown in FIGS. 4A-D), the expansion catheter system 3000 (shown in FIGS. 5A-E) or other implant retrieval system thereby can be utilized to engage one or more of the device retrieval members 1210 and to recapture, reposition, retrieve and/or remove the transcatheter pulmonary flow reduction device 1000 within the pulmonary artery 120 (shown in FIG. 1). Thereby, the device retrieval system 1200 advantageously can enable one-sided recapture, repositioning, retrieval and/or removal of the transcatheter pulmonary flow reduction device 1000. The transcatheter pulmonary flow reduction device 1000, in other words, can be recaptured, repositioned, retrieved and/or removed by engaging only the proximal frame end region 1100P of the device frame 1100 via the device retrieval system 1200. An ability to recapture, reposition, retrieve and/or remove the transcatheter pulmonary flow reduction device 1000 can help to eliminate a need for repeated surgical procedures and/or can allow for temporary and/or staged management of pulmonary flow.

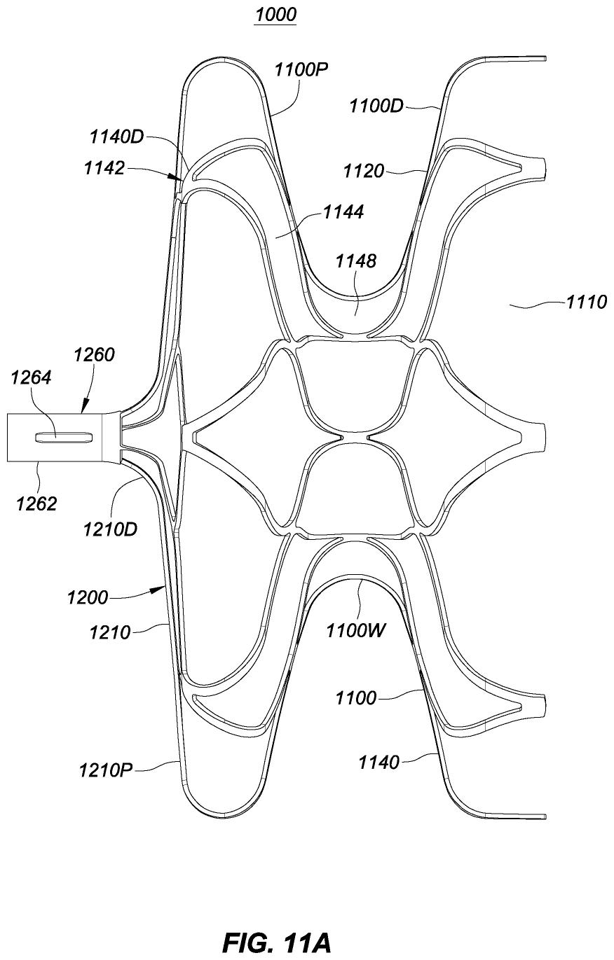

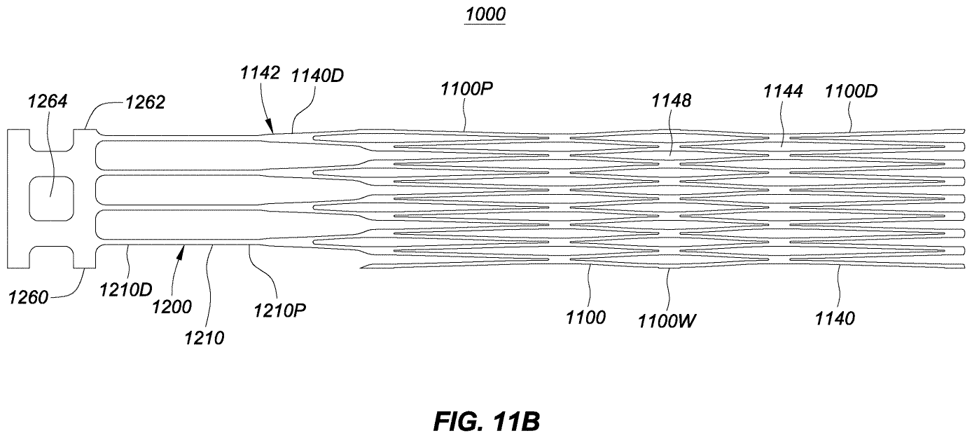

FIGS. 11A-B show yet another exemplary alternative embodiment of the transcatheter pulmonary flow reduction device 1000. The transcatheter pulmonary flow reduction device 1000 of FIGS. 11A-B can comprise an hourglass-shaped device frame 1100 with a central waist region 1100W disposed between proximal and distal frame end regions 1100P, 1100D and defining an internal channel 1110 in the manner discussed in more detail above with reference to the device frame 1100 shown in FIGS. 2, 3A-B and 6A-B. The external periphery 1120 of the proximal frame end region 1100P and the external periphery 1120 of the central waist region 1100W can define a first frame taper angle; whereas, the external periphery 1120 of the distal frame end region 1100F and the external periphery 1120 of the central waist region 1100W can define a second frame taper angle. Although preferably comprising uniform angles, the first and second frame taper angles can be different in selected embodiments and/or in selected applications.

The first and second frame taper angles can comprise any suitable angle, such as any predetermined angle within a taper angle range between twenty degrees and eighty degrees, or within any angle subrange of the taper angle range, without limitation. As illustrated in FIG. 11A, the first and second frame taper angles can comprise steep or large frame taper angles near an upper limit within the taper angle range. Exemplary large frame taper angles can include, but are not limited to, frame taper angles between sixty degrees and eighty degrees.

The large first and second frame taper angles can help to reduce an overall axial length of the device frame 1100 while maintaining adequate radial anchoring force at the proximal and distal frame end regions 1100P, 1100D. Minimizing an axial footprint of the transcatheter pulmonary flow reduction device 1000 advantageously can help to reduce a likelihood of jailing or otherwise obstructing pulmonary artery branches (not shown) adjacent to the pulmonary artery 120 (shown in FIG. 1), which can be a critical design consideration in left and right pulmonary arteries of neonatal patients. Additionally and/or alternatively, the large first and second frame taper angles can help to promote self-centering of the device frame 1100 during expansion and/or to enhance positional stability of the transcatheter pulmonary flow reduction device 1000 once deployed.

An axial length of the central waist region 1100W of the device frame 1100 illustrated in FIGS. 11A-B optionally can be less than respective axial lengths of the central waist regions 1100W of the device frames 1100 as shown and described with reference to FIGS. 3A-B, 6A-B, 7A-B, 8A-B, 9A-B and/or 10A-B. In other words, an axial length of the device frame struts 1140 at the central waist region 1100W of FIGS. 11A-B can be reduced relative to axial lengths of the device frame struts 1140 at the central waist regions 1100W as shown and described relative to the embodiments of the transcatheter pulmonary flow reduction device 1000 of FIGS. 3A-B, 6A-B, 7A-B, 8A-B, 9A-B and/or 10A-B. Reducing the axial length of the device frame struts 1140 at the central waist region 1100W advantageously can allow the diamond-shaped growth frame cells 1148 of the central waist region 1100W to assume a more expanded configuration in a relaxed or otherwise unconstrained state. The geometry of the reduced-length central waist region 1100W can help to enhance radial strength at the flow-restrictive region of the central waist region 1100W and/or to reduce recoil when the internal channel 1110 at the waist region 1100W is re-expanded after implantation from the first dimension DW (shown in FIG. 5A) to the second (or enlarged) internal dimension DWE (shown in FIG. 5E).

The transcatheter pulmonary flow reduction device 1000 can comprise a device frame 1100 with any suitable geometric pattern or other arrangement of the frame cells 1144, 1148. As shown in FIGS. 11A-B, for example, the device frame 1100 can comprise an eight-cell circumferential pattern with three longitudinal rows of diamond-shaped frame cells 1144, 1148. This geometric pattern of the frame cells 1144, 1148 advantageously can allow for thicker and/or wider strut geometries of the device frame struts 1140 while still maintaining sufficient crimpability for loading into a low-profile or otherwise appropriately-sized delivery catheter system 2000 (shown in FIGS. 4A-D). The increased strut thickness can enhance radial strength and/or can provide improved vessel engagement upon expansion. Additionally and/or alternatively, the distal strut end regions 1140D of the device frame 1100 can be curved or otherwise positioned away from the vessel wall or internal lumen surface 124 (shown in FIG. 5A) of the pulmonary artery 120 (shown in FIG. 1) for reducing risk of vessel trauma and/or to minimize a likelihood of tissue ingrowth. The thicker and/or wider strut geometries of the device frame struts 1140 of the device frame struts 1140 and/or the positioning of the distal strut end regions 1140D away from the vessel wall 124 can help to promote radial engagement between the device frame 1100 and the vessel wall 124 through broad strut-to-wall contact rather than discrete apical contact.

FIGS. 11A-B also illustrate yet another exemplary alternative embodiment of the device retrieval system 1200 for recapturing, repositioning, retrieving and/or removing the transcatheter pulmonary flow reduction device 1000 within the pulmonary artery 120 (shown in FIG. 1). The device retrieval system 1200 can be provided in the manner discussed in more detail above with reference to the device retrieval system 1200 of FIGS. 9A-B with the one or more device retrieval members 1210 being biased or otherwise extending radially inwardly toward a longitudinal axis of the internal channel 1110 defined by the device frame 1100. In the manner discussed above with reference to the tendrils of FIGS. 9A-B, for example, at least one of the device retrieval members 1210 of the device retrieval system 1200 of FIGS. 11A-B can be provided as tendrils (or struts) that can be formed from a flexible material and that have a slender size, shape, width, depth, cross-section or other dimension.

In selected embodiments, the distal strut end regions 1140D of the proximal frame end region 1100P can be arranged in pairs. Each pair of the distal strut end regions 1140D can converge into, or otherwise be associated with, a respective device retrieval member 1210 of the device retrieval system 1200. A number of device retrieval members 1210 of the device retrieval system 1200 thus can be reduced by associating the device retrieval members 1210 with pairs of the distal strut end regions 1140D, rather than separately with the individual distal strut end regions 1140D, while preserving the device recapturing, repositioning, retrieving and/or removing functionality. If the device frame 1100 comprises an eight-cell circumferential pattern, for example, the device frame 1100 can include four pairs of the distal strut end regions 1140D, which can converge into, or otherwise be associated with, four device retrieval members 1210.

The device retrieval members 1210 can extend proximally from the pairs of the distal strut end regions 1140D. The reduction in the number of device retrieval members 1210 of the device retrieval system 1200 advantageously can help to reduce obstruction of the internal channel 1110 at the waist region 1100W and/or to facilitate introduction of the expansion catheter system 3000 for re-expanding the internal channel 1110 at the waist region 1100W to the second (or enlarged) internal dimension DWE (shown in FIG. 5E) or otherwise adjusting the transcatheter pulmonary flow reduction device 1000 after implantation.

The device retrieval members 1210 can extend radially inwardly toward the longitudinal axis of the internal channel 1110 at any suitable angle, such as any predetermined angle between forty-five degrees and eighty degrees, without limitation. The deep-angle of the device retrieval members 1210 advantageously can help increase a spacing between adjacent device retrieval members 1210, thereby improving access to the central waist region 1100W of the device frame 1100 for post-implant expansion or other intraluminal adjustment procedures for the transcatheter pulmonary flow reduction device 1000. Additionally and/or alternatively, the deep-angle of the device retrieval members 1210 can reduce an axial length of the device retrieval system 1200. The reduced axial length of the device retrieval system 1200 advantageously can help to minimize an overall footprint of the transcatheter pulmonary flow reduction device 1000 within the pulmonary artery 120 (shown in FIG. 1) and/or to reduce a risk of obstructing or otherwise jailing branch vessels that are adjacent to the pulmonary artery 120.

As shown in FIGS. 11A-B, the device retrieval members 1210 of the device retrieval system 1200 can comprise deep-angled, unrestrained tendrils that can merge or otherwise converge at a coupling device (or member or means) 1260 for engaging the delivery catheter system 2000 (shown in FIGS. 4A-D), the expansion catheter system 3000 (shown in FIGS. 5A-E) or other implant retrieval system can be utilized to engage the device retrieval system 1200 for recapturing, repositioning, retrieving and/or removing the transcatheter pulmonary flow reduction device 1000 within the pulmonary artery 120). The coupling device 1260, in selected embodiments, can be provided as a hollow pin as illustrated in FIG. 11A. Stated somewhat differently, the coupling device 1260 can comprise a coupler member housing 1262 for defining a periphery of the coupling device 1260, wherein the coupler member housing 1262 optionally can define one or more elongated engagement (or retention) openings 1264 for enhancing an engagement with the delivery catheter system 2000, the expansion catheter system 3000 or other implant retrieval system. The delivery catheter system 2000, the expansion catheter system 3000 or other implant retrieval system can be utilized to engage the coupling device 1260 and to recapture, reposition, retrieve and/or remove the transcatheter pulmonary flow reduction device 1000 within the pulmonary artery 120.

The device retrieval system 1200 thereby can enable one-sided recapture, repositioning, retrieval and/or removal of the transcatheter pulmonary flow reduction device 1000. The transcatheter pulmonary flow reduction device 1000, in other words, can be recaptured, repositioned, retrieved and/or removed by engaging only the proximal frame end region 1100P of the device frame 1100 via the device retrieval system 1200. An ability to recapture, reposition, retrieve and/or remove the transcatheter pulmonary flow reduction device 1000 can help to eliminate a need for repeated surgical procedures and/or can allow for temporary and/or staged management of pulmonary flow.

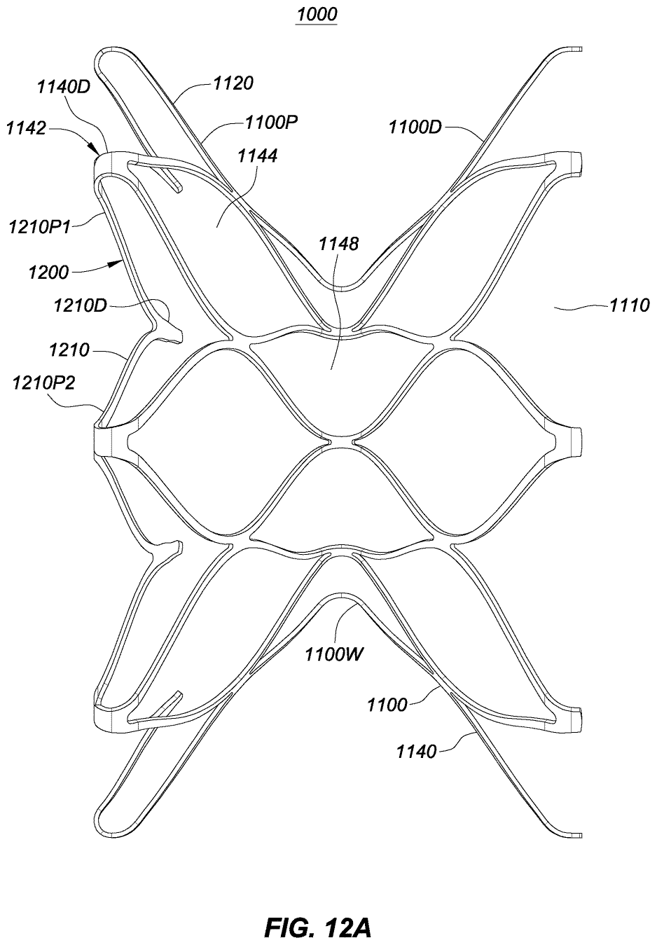

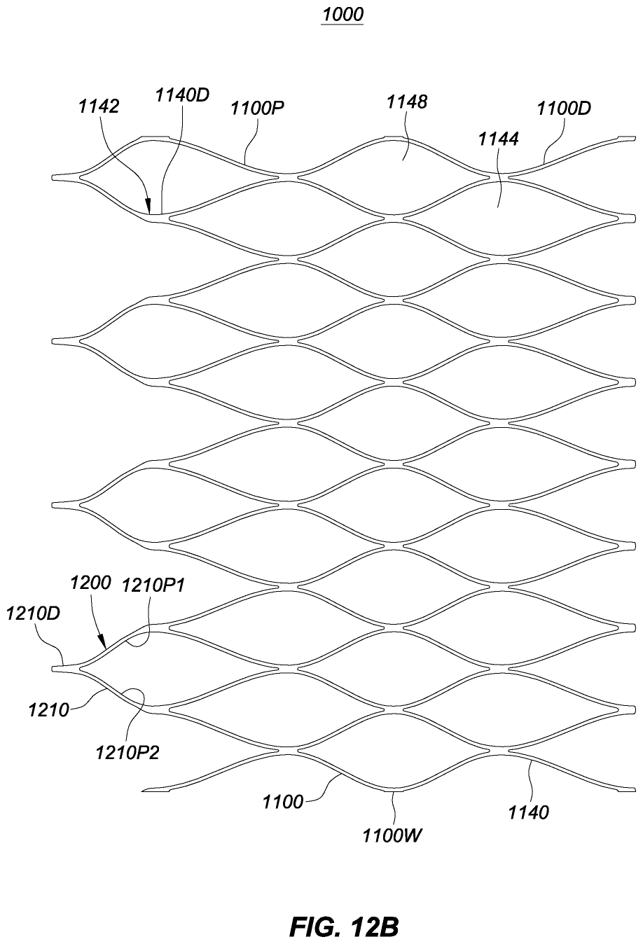

Turning to FIGS. 12A-B, a further exemplary alternative embodiment of the transcatheter pulmonary flow reduction device 1000 is shown. The transcatheter pulmonary flow reduction device 1000 can comprise an hourglass-shaped device frame 1100 with a central waist region 1100W disposed between proximal and distal frame end regions 1100P, 1100D and defining an internal channel 1110 in the manner discussed in more detail above with reference to the device frame 1100 shown in FIGS. 2, 3A-B and 6A-B. The external periphery 1120 of the proximal frame end region 1100P and the external periphery 1120 of the central waist region 1100W can define a first frame taper angle; whereas, the external periphery 1120 of the distal frame end region 1100F and the external periphery 1120 of the central waist region 1100W can define a second frame taper angle. The first and second frame taper angles can comprise any suitable angle, such as any predetermined angle within a taper angle range between twenty degrees and eighty degrees, or within any angle subrange of the taper angle range, without limitation. Although preferably comprising uniform angles, the first and second frame taper angles can be different in selected embodiments and/or in selected applications.

The transcatheter pulmonary flow reduction device 1000 can comprise a device frame 1100 with any suitable geometric pattern or other arrangement of the frame cells 1144, 1148. As shown in FIGS. 12A-B, for example, the device frame 1100 can comprise an eight-cell circumferential pattern with three longitudinal rows of diamond-shaped frame cells 1144, 1148. This geometric pattern of the frame cells 1144, 1148 advantageously can allow for thicker and/or wider strut geometries of the device frame struts 1140 while still maintaining sufficient crimpability for loading into a low-profile or otherwise appropriately-sized delivery catheter system 2000 (shown in FIGS. 4A-D). The increased strut thickness can enhance radial strength and/or can provide improved vessel engagement upon expansion.

Additionally and/or alternatively, the distal strut end regions 1140D of the device frame 1100 can be curved or otherwise positioned away from the vessel wall or internal lumen surface 124 (shown in FIG. 5A) of the pulmonary artery 120 (shown in FIG. 1) for reducing risk of vessel trauma and/or to minimize a likelihood of tissue ingrowth. The distal strut end regions 1140D, in other words, can be biased or otherwise extend radially inwardly toward a longitudinal axis of the internal channel 1110 defined by the device frame 1100. The thicker and/or wider strut geometries of the device frame struts 1140 of the device frame struts 1140 and/or the positioning of the distal strut end regions 1140D away from the vessel wall 124 can help to promote radial engagement between the device frame 1100 and the vessel wall 124 through broad strut-to-wall contact rather than discrete apical contact.

The transcatheter pulmonary flow reduction device 1000 is shown as being associated with a device retrieval system 1200. The device retrieval system can comprise a plurality of device retrieval members 1210 each having a distal retrieval member end region 1210D that extends radially inwardly toward a longitudinal axis of the internal channel 1110 defined by the transcatheter pulmonary flow reduction device 1000. As shown in FIGS. 12A-B, for example, the device retrieval members 1210 are illustrated as extending radially inwardly from respective pairs of distal strut end regions 1140D of the proximal frame end region 1100P of the device frame 1100. The distal strut end regions 1140D (or frame end junctions 1142) of the proximal frame end region 1100P, in other words, can be grouped into pairs, and each device retrieval member 1210 can extend radially inwardly from the paired distal strut end regions 1140D.

Each pair of adjacent distal strut end regions 1140D advantageously can converge to form a single device retrieval member 1210. In selected embodiments, the device retrieval members 1210 can include first and second proximal retrieval member end regions 1210P1, 1210P2 and a distal retrieval member end region 1210D. The first proximal retrieval member end region 1210P1 can be configured to intersect or otherwise cooperate with a first distal strut end region 1140D of the paired distal strut end regions 1140D; whereas, the second proximal retrieval member end region 1210P2 can be configured to intersect or otherwise cooperate with a second distal strut end region 1140D of the same paired distal strut end regions 1140D. The distal retrieval member end region 1210D can be figured to extend from the paired distal strut end regions 1140D and radially inwardly toward the longitudinal axis of the internal channel 1110.

A number of device retrieval members 1210 of the device retrieval system 1200 advantageously can be reduced by associating the device retrieval members 1210 with pairs of the distal strut end regions 1140D, rather than separately with the individual distal strut end regions 1140D, while preserving the device recapturing, repositioning, retrieving and/or removing functionality. If the device frame 1100 comprises an eight-cell circumferential pattern, for example, the device frame 1100 can include four pairs of the distal strut end regions 1140D, which can converge into, or otherwise be associated with, four device retrieval members 1210.

The radially inwardly extending geometry of the device retrieval members 1210 thereby can create four discrete capture or snaring features positioned along the external periphery 1120 of the proximal frame end region 1100P. Each device retrieval member 1210 can form a curved or angled structure that projects partially into the internal channel 1110 of the transcatheter pulmonary flow reduction device 1000, providing multiple grasp points that are accessible to the delivery catheter system 2000 (shown in FIGS. 4A-D), the expansion catheter system 3000 (shown in FIGS. 5A-E) or other implant retrieval system. The radially inwardly extending device retrieval members 1210, for example, can be engaged by implant retrieval systems such as En Snare retrieval systems, micro forceps, wire-loop snares or other grasping tools introduced through a transcatheter approach. Additionally and/or alternatively, the radially inwardly extending device retrieval members 1210 can enable controlled repositioning, partial recapture and/or full retrieval of the transcatheter pulmonary flow reduction device 1000 during the index procedure or at later follow-up stages.

The paired distal strut end regions 1140D and the radially inwardly extending device retrieval members 1210 can help to avoid obstructions within the internal channel 1110 of the transcatheter pulmonary flow reduction device 1000, avoid obstructions associated with the central waist region 1100W and/or allow clear access for any implant retrieval system introduced for controlling the adjustable dimension DW of the internal channel 1110 at the central waist region 1100W. The device retrieval system 1200 of FIGS. 12A-B advantageously can provide and otherwise maintain a simplified proximal profile while retaining reliable retrievability. The device retrieval members 1210 can be positioned to remain compact during crimping and delivery but to expand or rebound to provide the radially inwardly extending geometry after deployment, providing predictable and easily targeted snaring features for retrieval. The radially inwardly extending device retrieval members 1210 can be provided in any suitable manner and formed from any appropriate material. In selected embodiments, the radially inwardly extending device retrieval members 1210 can be manufactured or otherwise formed from a material, such as Nitinol, which exhibits a shape-memory behavior.

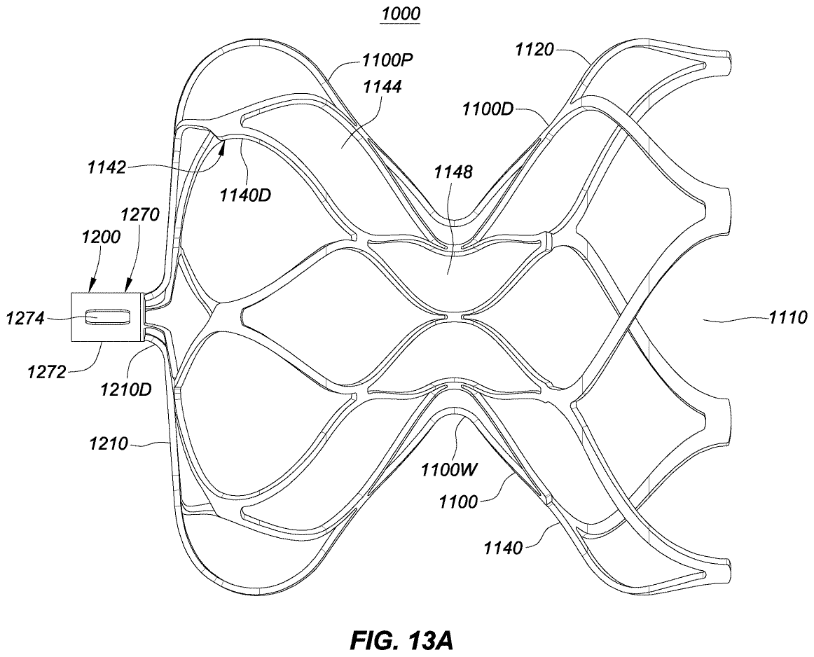

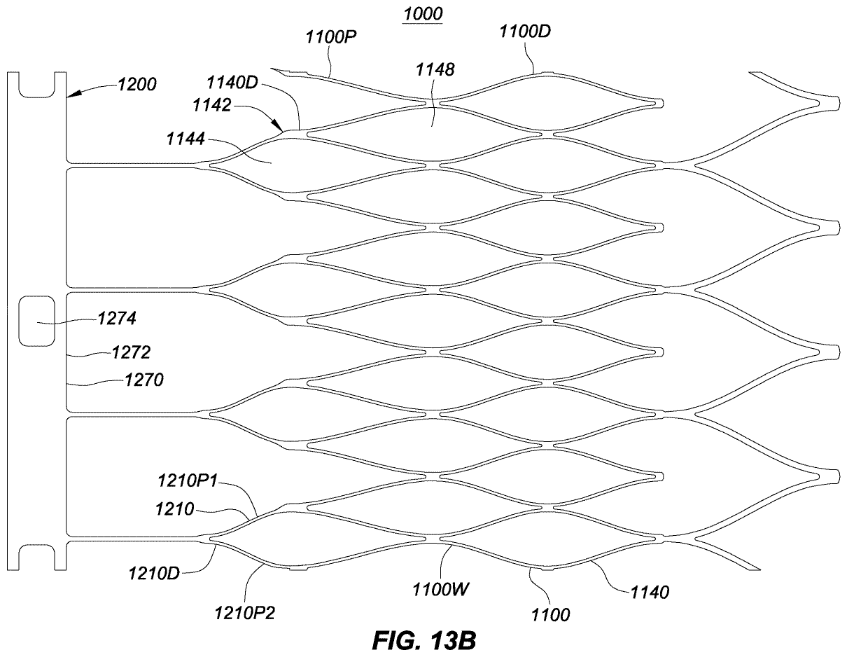

Another further exemplary alternative embodiment of the transcatheter pulmonary flow reduction device of the transcatheter pulmonary flow reduction device 1000 is illustrated in FIGS. 13A-B. The transcatheter pulmonary flow reduction device 1000 can comprise an hourglass-shaped device frame 1100 with a central waist region 1100W disposed between proximal and distal frame end regions 1100P, 1100D and defining an internal channel 1110 in the manner discussed in more detail above with reference to the device frame 1100 shown in FIGS. 2, 3A-B and 6A-B. The external periphery 1120 of the proximal frame end region 1100P and the external periphery 1120 of the central waist region 1100W can define a first frame taper angle; whereas, the external periphery 1120 of the distal frame end region 1100F and the external periphery 1120 of the central waist region 1100W can define a second frame taper angle. The first and second frame taper angles can comprise any suitable angle, such as any predetermined angle within a taper angle range between twenty degrees and eighty degrees, or within any angle subrange of the taper angle range, without limitation. Although preferably comprising uniform angles, the first and second frame taper angles can be different in selected embodiments and/or in selected applications.

The transcatheter pulmonary flow reduction device 1000 can comprise a device frame 1100 with any suitable geometric pattern or other arrangement of the frame cells 1144, 1148. As shown in FIGS. 13A-B, for example, the device frame 1100 can comprise an eight-cell circumferential pattern with three longitudinal rows of diamond-shaped frame cells 1144, 1148. This geometric pattern of the frame cells 1144, 1148 advantageously can allow for thicker and/or wider strut geometries of the device frame struts 1140 while still maintaining sufficient crimpability for loading into a low-profile or otherwise appropriately-sized delivery catheter system 2000 (shown in FIGS. 4A-D). The increased strut thickness can enhance radial strength and/or can provide improved vessel engagement upon expansion. In selected embodiments, the distal frame end region 1100D of the device frame 1100 can comprise a predetermined cell configuration with larger alternating half cells extending from the distal frame end region 1100D.

The distal strut end regions 1140D of the device frame 1100 optionally can be curved or otherwise positioned away from the vessel wall or internal lumen surface 124 (shown in FIG. 5A) of the pulmonary artery 120 (shown in FIG. 1) for reducing risk of vessel trauma and/or to minimize a likelihood of tissue ingrowth. The distal strut end regions 1140D, in other words, can be biased or otherwise extend radially inwardly toward a longitudinal axis of the internal channel 1110 defined by the device frame 1100. The thicker and/or wider strut geometries of the device frame struts 1140 of the device frame struts 1140 and/or the positioning of the distal strut end regions 1140D away from the vessel wall 124 can help to promote radial engagement between the device frame 1100 and the vessel wall 124 through broad strut-to-wall contact rather than discrete apical contact.

As shown in FIGS. 13A-B, the transcatheter pulmonary flow reduction device 1000 can be associated with a device retrieval system 1200. The device retrieval system can comprise a plurality of device retrieval members 1210 each having a distal retrieval member end region 1210D that extends radially inwardly toward a longitudinal axis of the internal channel 1110 defined by the transcatheter pulmonary flow reduction device 1000. The device retrieval members 1210 are illustrated in FIGS. 13A-B as extending radially inwardly from respective pairs of distal strut end regions 1140D of the proximal frame end region 1100P of the device frame 1100. The distal strut end regions 1140D (or frame end junctions 1142) of the proximal frame end region 1100P, in other words, can be grouped into pairs, and each device retrieval member 1210 can extend radially inwardly from the paired distal strut end regions 1140D.

Each pair of adjacent distal strut end regions 1140D advantageously can converge to form a single device retrieval member 1210. In selected embodiments, the device retrieval members 1210 can include first and second proximal retrieval member end regions 1210P1, 1210P2 and a distal retrieval member end region 1210D. The first proximal retrieval member end region 1210P1 can be configured to intersect or otherwise cooperate with a first distal strut end region 1140D of the paired distal strut end regions 1140D; whereas, the second proximal retrieval member end region 1210P2 can be configured to intersect or otherwise cooperate with a second distal strut end region 1140D of the same paired distal strut end regions 1140D. The distal retrieval member end region 1210D can be figured to extend from the paired distal strut end regions 1140D and radially inwardly toward the longitudinal axis of the internal channel 1110.

A number of device retrieval members 1210 of the device retrieval system 1200 advantageously can be reduced by associating the device retrieval members 1210 with pairs of the distal strut end regions 1140D, rather than separately with the individual distal strut end regions 1140D, while preserving the device recapturing, repositioning, retrieving and/or removing functionality. If the device frame 1100 comprises an eight-cell circumferential pattern, for example, the device frame 1100 can include four pairs of the distal strut end regions 1140D, which can converge into, or otherwise be associated with, four device retrieval members 1210.

The device retrieval system 1200 thereby can enable one-sided recapture, repositioning, retrieval and/or removal of the transcatheter pulmonary flow reduction device 1000. The transcatheter pulmonary flow reduction device 1000, in other words, can be recaptured, repositioned, retrieved and/or removed by engaging only the proximal frame end region 1100P of the device frame 1100 via the device retrieval system 1200. An ability to recapture, reposition, retrieve and/or remove the transcatheter pulmonary flow reduction device 1000 can help to eliminate a need for repeated surgical procedures and/or can allow for temporary and/or staged management of pulmonary flow.

By reducing the number of device retrieval members 1210, the proximal device frame lobe 1130P can be formed or otherwise provided with device frame struts 1140 that have an increased strut thickness and/or an increased strut width while maintaining sufficient crimpability to be disposed on a low-profile or otherwise appropriately-sized delivery catheter system 2000. The increased strut thickness and/or the increased strut width can help to reduce cell deflection during pulsatile loading, thereby enhancing radial engagement and improving vessel retention at the distal frame end region 1100D. Since the distal frame end region 1100D primarily provides anchoring while the proximal frame end region 1100P provides sealing, the strengthened distal structure increases overall positional stability without compromising deliverability or adjustability of the internal dimension DW of the internal channel 1110 at the waist region 1100W.

As shown in FIGS. 13A-B, the device retrieval members 1210 of the device retrieval system 1200 can comprise deep-angled, unrestrained tendrils that can merge or otherwise converge at a coupling device (or member or means) 1270 for engaging the delivery catheter system 2000 (shown in FIGS. 4A-D), the expansion catheter system 3000 (shown in FIGS. 5A-E) or other implant retrieval system can be utilized to engage the device retrieval system 1200 for recapturing, repositioning, retrieving and/or removing the transcatheter pulmonary flow reduction device 1000 within the pulmonary artery 120 (shown in FIG. 1). The coupling device 1270, in selected embodiments, can be provided as a hollow pin as illustrated in FIG. 13A. Stated somewhat differently, the coupling device 1270 can comprise a coupler member housing 1272 for defining a periphery of the coupling device 1270. Advantageously, the coupling device 1270 can provide a dedicated coupling interface for recapture, repositioning, retrieval and/or removal of the transcatheter pulmonary flow reduction device 1000.

The coupling device 1270, as shown in FIGS. 13A-B, optionally can define one or more engagement (or retention) slots, windows or other openings 1274 for enhancing an engagement with the delivery catheter system 2000, the expansion catheter system 3000 or other implant retrieval system. The delivery catheter system 2000, the expansion catheter system 3000 or other implant retrieval system can be utilized to engage the coupling device 1270 and to recapture, reposition, retrieve and/or remove the transcatheter pulmonary flow reduction device 1000 within the pulmonary artery 120. Each of the engagement openings 1274 can have a predetermined geometric profile. Exemplary geometric profiles of the engagement openings 1274 can include, but are not limited to, a rectangular profile and/or a square profile. The engagement openings 1274 can be provided in any suitable manner, such as via laser-cutting opposing sides of the coupler member housing 1272.

In selected embodiments, the engagement openings 1274 can be dimensioned for mechanically interfacing with a complementary interface engagement device (or member) 2340 (shown in FIGS. 26 and 27A-B) disposed, for example, at the catheter distal end region 2100 (shown in FIGS. 4A-D) of the delivery catheter system 2000 (shown in FIGS. 4A-D), the expansion catheter system 3000 (shown in FIGS. 5A-E) or other implant retrieval system. The catheter distal end region 2100 (shown in FIGS. 4A-D) of the implant retrieval system, for example, can clip, lock, expand or otherwise engage the engagement openings 1274 to initiate and/or maintain a (temporary) mechanical connection with the transcatheter pulmonary flow reduction device 1000 during advancement, positioning, partial deployment, repositioning, retrieval, recapture and/or removal of the transcatheter pulmonary flow reduction device 1000. After the transcatheter pulmonary flow reduction device 1000 has been positioned and the desired degree of pulmonary flow reduction is confirmed, the coupling between the transcatheter pulmonary flow reduction device 1000 and the implant retrieval system can be terminated, allowing controlled decoupling between the transcatheter pulmonary flow reduction device 1000 and the implant retrieval system.

The engagement openings 1274 optionally can facilitate retrieval and/or recapture of the transcatheter pulmonary flow reduction device 1000. The engagement openings 1274 defined by the coupler member housing 1272, for example, can allow the implant retrieval system, such as a snare (not shown), a gooseneck snare (not shown), a suture-loop snare (not shown) and/or a wire-loop capture device(not shown), to securely engage the coupling device 1270 for acute repositioning and/or chronic removal of the transcatheter pulmonary flow reduction device 1000 from the pulmonary artery 120. The coupling device 1270 advantageously can enable reliable engagement between the coupling device 1270 and the implant retrieval system from multiple approach angles and/or can reduce dependency on tendril-tip capture alone. The coupling device 1270 thereby can provide a robust dual-purpose interface for coupling during deployment and/or retrieval during follow-up procedures.

The device retrieval system 1200 thereby can enable one-sided recapture, repositioning, retrieval and/or removal of the transcatheter pulmonary flow reduction device 1000. The transcatheter pulmonary flow reduction device 1000, in other words, can be recaptured, repositioned, retrieved and/or removed by engaging only the proximal frame end region 1100P of the device frame 1100 via the device retrieval system 1200. An ability to recapture, reposition, retrieve and/or remove the transcatheter pulmonary flow reduction device 1000 can help to eliminate a need for repeated surgical procedures and/or can allow for temporary and/or staged management of pulmonary flow.

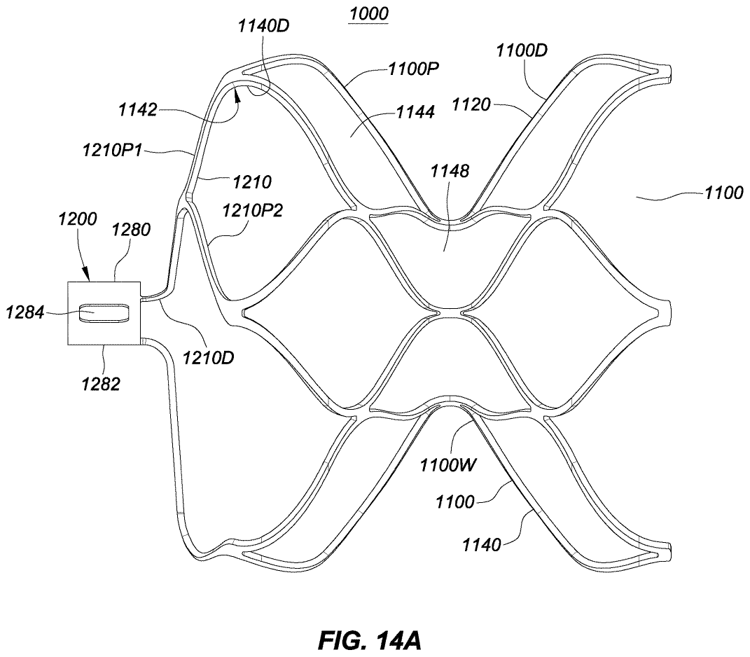

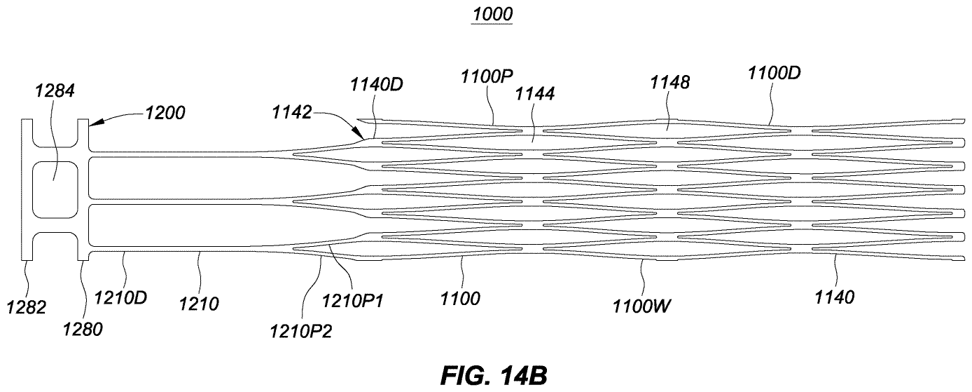

Still another further exemplary alternative embodiment of the transcatheter pulmonary flow reduction device 1000 is illustrated in FIGS. 14A-B. Turning to FIGS. 14A-B, the transcatheter pulmonary flow reduction device 1000 can comprise an hourglass-shaped device frame 1100 with a central waist region 1100W disposed between proximal and distal frame end regions 1100P, 1100D and defining an internal channel 1110 in the manner discussed in more detail above with reference to the device frame 1100 shown in FIGS. 2, 3A-B and 6A-B. The external periphery 1120 of the proximal frame end region 1100P and the external periphery 1120 of the central waist region 1100W can define a first frame taper angle; whereas, the external periphery 1120 of the distal frame end region 1100F and the external periphery 1120 of the central waist region 1100W can define a second frame taper angle. The first and second frame taper angles can comprise any suitable angle, such as any predetermined angle within a taper angle range between twenty degrees and eighty degrees, or within any angle subrange of the taper angle range, without limitation. Although preferably comprising uniform angles, the first and second frame taper angles can be different in selected embodiments and/or in selected applications.

The transcatheter pulmonary flow reduction device 1000 can comprise a device frame 1100 with any suitable geometric pattern or other arrangement of the frame cells 1144, 1148. As shown in FIGS. 14A-B, for example, the device frame 1100 can comprise a six-cell circumferential pattern with three longitudinal rows of diamond-shaped frame cells 1144, 1148. Relative to device frames 1100 with circumferential patterns comprising eight or more frame cells 1144, 1148, the geometric pattern of frame cells 1144, 1148 shown in FIGS. 14A-B advantageously can allow for still thicker and/or wider strut geometries of the device frame struts 1140 while still maintaining sufficient crimpability for loading into a low-profile or otherwise appropriately-sized delivery catheter system 2000 (shown in FIGS. 4A-D). For example, the device frame 1100 can be compatible for loading into a low-profile delivery catheter system 2000 suitable for neonatal and other pediatric applications. The thicker strut construction advantageously can provide enhanced radial engagement with the vessel wall or internal lumen surface 124 (shown in FIG. 5A) of the pulmonary artery 120 (shown in FIG. 1) and improved resistance to deformation under pulsatile loading. The further-increased strut thickness optionally can enhance radial strength and/or can provide improved vessel engagement upon expansion.

The distal strut end regions 1140D of the device frame 1100 optionally can be curved or otherwise positioned away from the vessel wall (not shown) of the pulmonary artery 120 for reducing risk of vessel trauma and/or to minimize a likelihood of tissue ingrowth. The distal strut end regions 1140D, in other words, can be biased or otherwise extend radially inwardly toward a longitudinal axis of the internal channel 1110 defined by the device frame 1100. The thicker and/or wider strut geometries of the device frame struts 1140 of the device frame struts 1140 and/or the positioning of the distal strut end regions 1140D away from the vessel wall 124 can help to promote radial engagement between the device frame 1100 and the vessel wall 124 through broad strut-to-wall contact rather than discrete apical contact.

As shown in FIGS. 14A-B, the transcatheter pulmonary flow reduction device 1000 can be associated with a device retrieval system 1200. The device retrieval system can comprise a plurality of device retrieval members 1210 each having a distal retrieval member end region 1210D that extends radially inwardly toward a longitudinal axis of the internal channel 1110 defined by the transcatheter pulmonary flow reduction device 1000. In selected embodiments, one or more of the device retrieval members 1210 can comprise shape-angled device retrieval members. The device retrieval members 1210 are illustrated in FIGS. 14A-B as extending radially inwardly from respective pairs of distal strut end regions 1140D of the proximal frame end region 1100P of the device frame 1100. The distal strut end regions 1140D (or frame end junctions 1142) of the proximal frame end region 1100P, in other words, can be grouped into pairs, and each device retrieval member 1210 can extend radially inwardly from the paired distal strut end regions 1140D.

Each pair of adjacent distal strut end regions 1140D advantageously can converge to form a single device retrieval member 1210. In selected embodiments, the device retrieval members 1210 can include first and second proximal retrieval member end regions 1210P1, 1210P2 and a distal retrieval member end region 1210D. The first proximal retrieval member end region 1210P1 can be configured to intersect or otherwise cooperate with a first distal strut end region 1140D of the paired distal strut end regions 1140D; whereas, the second proximal retrieval member end region 1210P2 can be configured to intersect or otherwise cooperate with a second distal strut end region 1140D of the same paired distal strut end regions 1140D. The distal retrieval member end region 1210D can be figured to extend from the paired distal strut end regions 1140D and radially inwardly toward the longitudinal axis of the internal channel 1110.

A number of device retrieval members 1210 of the device retrieval system 1200 advantageously can be reduced by associating the device retrieval members 1210 with pairs of the distal strut end regions 1140D, rather than separately with the individual distal strut end regions 1140D, while preserving the device recapturing, repositioning, retrieving and/or removing functionality. If the device frame 1100 comprises a six-cell circumferential pattern, for example, the device frame 1100 can include three pairs of the distal strut end regions 1140D, which can converge into, or otherwise be associated with, four device retrieval members 1210.

The device retrieval system 1200 thereby can enable one-sided recapture, repositioning, retrieval and/or removal of the transcatheter pulmonary flow reduction device 1000. The transcatheter pulmonary flow reduction device 1000, in other words, can be recaptured, repositioned, retrieved and/or removed by engaging only the proximal frame end region 1100P of the device frame 1100 via the device retrieval system 1200. An ability to recapture, reposition, retrieve and/or remove the transcatheter pulmonary flow reduction device 1000 can help to eliminate a need for repeated surgical procedures and/or can allow for temporary and/or staged management of pulmonary flow.

The reduction in the number of device retrieval members 1210 of the device retrieval system 1200 advantageously can help to reduce obstruction of the internal channel 1110 at the waist region 1100W and/or to facilitate introduction of the expansion catheter system 3000 for re-expanding the internal channel 1110 at the waist region 1100W to the second (or enlarged) internal dimension DWE (shown in FIG. 5E) or otherwise adjusting the transcatheter pulmonary flow reduction device 1000 after implantation. The geometric arrangement of the device retrieval members 1210 likewise can provide a simplified mechanism for coupling an implant retrieval system with the coupling device 1270 of the device retrieval system 1200 while maintaining reliable engagement and/or recapture ability. The transcatheter pulmonary flow reduction device 1000 thereby can balance structure reinforcement from the thicker and/or wider strut geometries of the device frame struts 1140 with improved lumen accessibility and/or a reduced complexity at the profile of the proximal frame end region 1100P.

As shown in FIGS. 14A-B, the device retrieval members 1210 of the device retrieval system 1200 can comprise deep-angled, unrestrained tendrils that can merge or otherwise converge at a coupling device (or member or means) 1270 for engaging the delivery catheter system 2000 (shown in FIGS. 4A-D), the expansion catheter system 3000 (shown in FIGS. 5A-E) or other implant retrieval system can be utilized to engage the device retrieval system 1200 for recapturing, repositioning, retrieving and/or removing the transcatheter pulmonary flow reduction device 1000 within the pulmonary artery 120. The coupling device 1270, in selected embodiments, can be provided as a hollow pin as illustrated in FIG. 14A. Stated somewhat differently, the coupling device 1270 can comprise a coupler member housing 1272 for defining a periphery of the coupling device 1270. Advantageously, the coupling device 1270 can provide a dedicated coupling interface for recapture, repositioning, retrieval and/or removal of the transcatheter pulmonary flow reduction device 1000.

The coupling device 1270, as shown in FIGS. 14A-B, optionally can define one or more engagement (or retention) slots, windows or other openings 1274 for enhancing an engagement with the delivery catheter system 2000, the expansion catheter system 3000 or other implant retrieval system. The delivery catheter system 2000, the expansion catheter system 3000 or other implant retrieval system can be utilized to engage the coupling device 1270 and to recapture, reposition, retrieve and/or remove the transcatheter pulmonary flow reduction device 1000 within the pulmonary artery 120. Each of the engagement openings 1274 can have a predetermined geometric profile. Exemplary geometric profiles of the engagement openings 1274 can include, but are not limited to, a rectangular profile and/or a square profile. The engagement openings 1274 can be provided in any suitable manner, such as via laser-cutting opposing sides of the coupler member housing 1272.

In selected embodiments, the engagement openings 1274 can be dimensioned for mechanically interfacing with a complementary interface engagement device (or member) 2340 (shown in FIGS. 26 and 27A-B) disposed, for example, at the catheter distal end region 2100 (shown in FIGS. 4A-D) of the delivery catheter system 2000 (shown in FIGS. 4A-D), the expansion catheter system 3000 (shown in FIGS. 5A-E) or other implant retrieval system. The catheter distal end region 2100 (shown in FIGS. 4A-D) of the implant retrieval system, for example, can clip, lock, expand or otherwise engage the engagement openings 1274 to initiate and/or maintain a (temporary) mechanical connection with the transcatheter pulmonary flow reduction device 1000 during advancement, positioning, partial deployment, repositioning, retrieval, recapture and/or removal of the transcatheter pulmonary flow reduction device 1000. After the transcatheter pulmonary flow reduction device 1000 has been positioned and the desired degree of pulmonary flow reduction is confirmed, the coupling between the transcatheter pulmonary flow reduction device 1000 and the implant retrieval system can be terminated, allowing controlled decoupling between the transcatheter pulmonary flow reduction device 1000 and the implant retrieval system.

The engagement openings 1274 optionally can facilitate retrieval and/or recapture of the transcatheter pulmonary flow reduction device 1000. The engagement openings 1274 defined by the coupler member housing 1272, for example, can allow the implant retrieval system, such as a snare (not shown), a gooseneck snare (not shown), a suture-loop snare (not shown) and/or a wire-loop capture device(not shown), to securely engage the coupling device 1270 for acute repositioning and/or chronic removal of the transcatheter pulmonary flow reduction device 1000 from the pulmonary artery 120. The coupling device 1270 advantageously can enable reliable engagement between the coupling device 1270 and the implant retrieval system from multiple approach angles and/or can reduce dependency on tendril-tip capture alone. The coupling device 1270 thereby can provide a robust dual-purpose interface for coupling during deployment and/or retrieval during follow-up procedures.

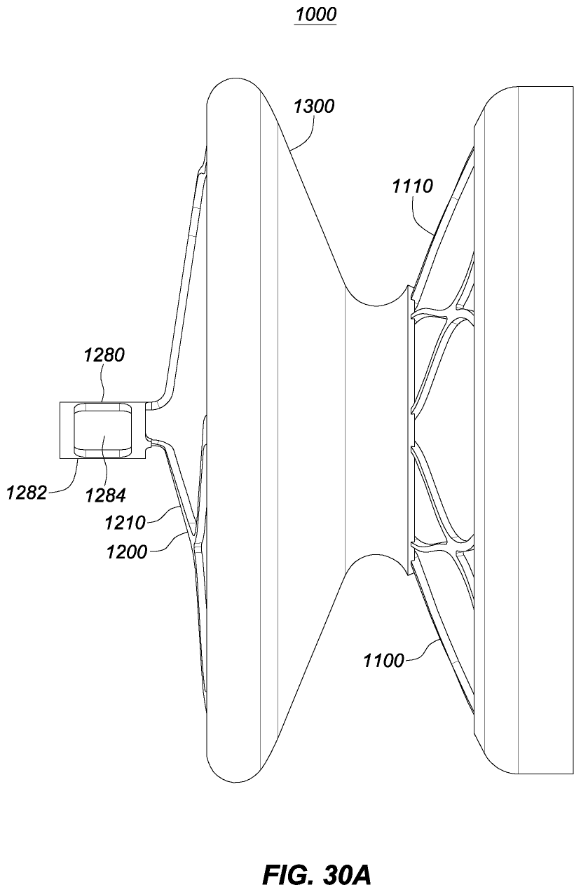

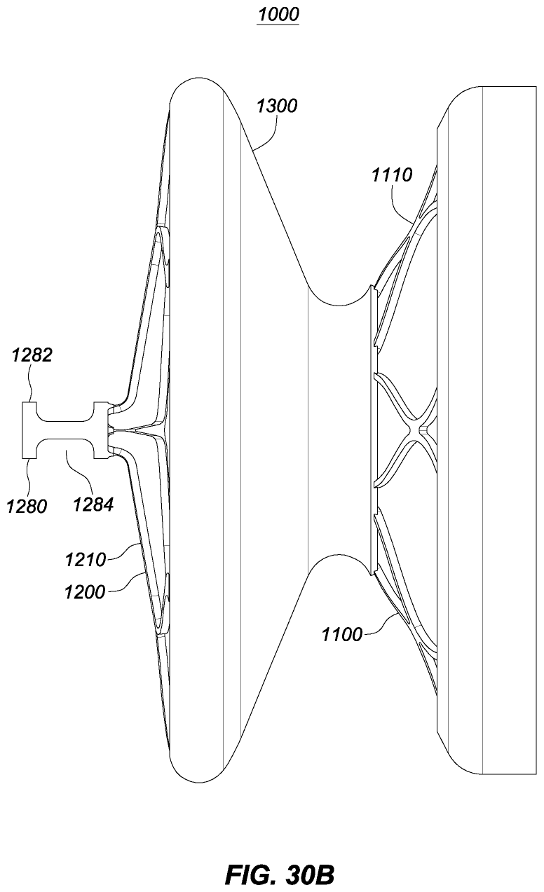

Turning to FIGS. 30A-B, the coupling device 1280 can comprise a proximal retention pin (or hollow pin). The coupling device 1280, in other words, can be provided as a hollow pin. Stated somewhat differently, the coupling device 1280 can comprise a coupler member housing 1282 for defining a periphery of the coupling device 1280. Advantageously, the coupling device 1280 can provide a dedicated coupling interface for recapture, repositioning, retrieval and/or removal of the transcatheter pulmonary flow reduction device 1000. The coupling device 1280 optionally can define one or more engagement (or retention) slots, windows or other openings 1284 for enhancing an engagement with the delivery catheter system 2000 (shown in FIGS. 4A-D), the expansion catheter system 3000 (shown in FIGS. 5A-E) or other implant retrieval system. Stated somewhat differently, the coupling device 1280 may define one or more slots (or windows) 1284 formed through the coupler member housing 1282. The dimensions of the openings 1284 may vary in width, length, and circumferential position. In certain configurations, for example, the openings 1284 can be dimensioned to be sufficiently wide and elongated to facilitate reliable recapture of the transcatheter pulmonary flow reduction device 1000 into a catheter or sheath. Elongated window geometries advantageously can help to reduce a likelihood of the pin entering a distal tip of a sheath or catheter in a transverse or “T” orientation that could cause jamming or incomplete recapture. Instead, the slot geometry can help to promote a linear alignment of the pin with the catheter lumen, enabling smooth, axial re-entry during retrieval. An outer diameter of the coupler member housing 1282 optionally can be selected to be compatible with low-profile delivery and retrieval systems, including off-the-shelf four French catheters, custom four French catheters and/or four French sheaths, while maintaining sufficient structural integrity for coupling and snaring.

The device retrieval system 1200 thereby can enable one-sided recapture, repositioning, retrieval and/or removal of the transcatheter pulmonary flow reduction device 1000. The transcatheter pulmonary flow reduction device 1000, in other words, can be recaptured, repositioned, retrieved and/or removed by engaging only the proximal frame end region 1100P of the device frame 1100 via the device retrieval system 1200. An ability to recapture, reposition, retrieve and/or remove the transcatheter pulmonary flow reduction device 1000 can help to eliminate a need for repeated surgical procedures and/or can allow for temporary and/or staged management of pulmonary flow.

In selected embodiments, the transcatheter pulmonary flow reduction device 1000 optionally can include one or more radiopaque or other contrast-enhancing coatings or other markers (not shown) for enhancing visualization of the transcatheter pulmonary flow reduction device 1000 under fluoroscopy, Computed Tomography (or CT) imaging or other radiographic imaging modalities. The radiopaque markers can be formed or otherwise provided by any suitable marker material. Exemplary suitable marker materials can include, but are not limited to, tantalum, platinum, platinum-iridium alloys, gold and/or tungsten.

The radiopaque markers can be provided as one or more discrete marker bands and/or can be disposed on the transcatheter pulmonary flow reduction device 1000 in any suitable manner. For example, the radiopaque markers can be crimped and/or laser-welded to the device frame 1100. Additionally and/or alternatively, the radiopaque markers can be applied as thin-film coatings at selected regions of the transcatheter pulmonary flow reduction device 1000. The radiopaque markers, for instance, can be disposed at (or adjacent to) the proximal frame end region 1100P, the central waist region 1100W, the distal frame end region 1100D, the device retrieval system 1200 and/or one or more functional coupling components, such as the coupling device 1240, 1260, 1270, 1280 (shown in FIGS. 9A-B, 11A-B, 13A-B and 30A-B) of the device retrieval system 1200. In selected embodiments, the radiopaque markers can be disposed at (or adjacent to) at least one of the device frame struts 1140 of the proximal frame end region 1100P, the central waist region 1100W and/or the distal frame end region 1100D and/or at least one of the device retrieval members 1210 of the device retrieval system 1200.

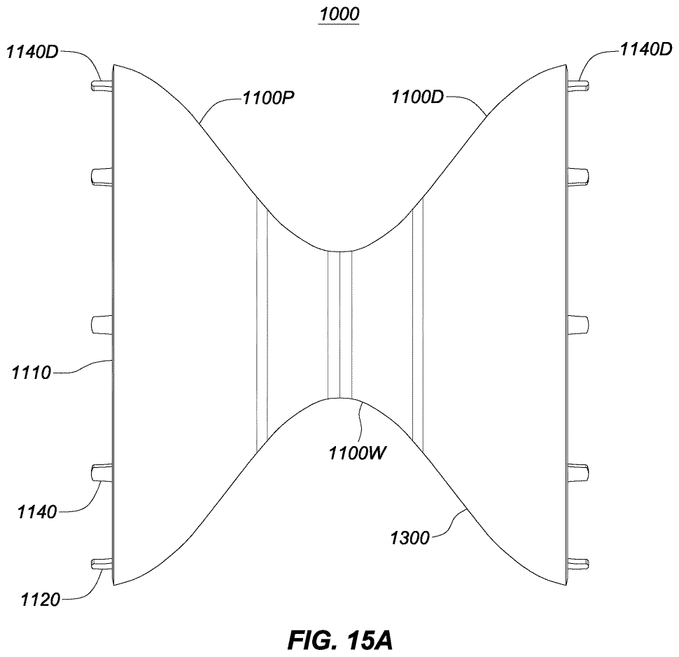

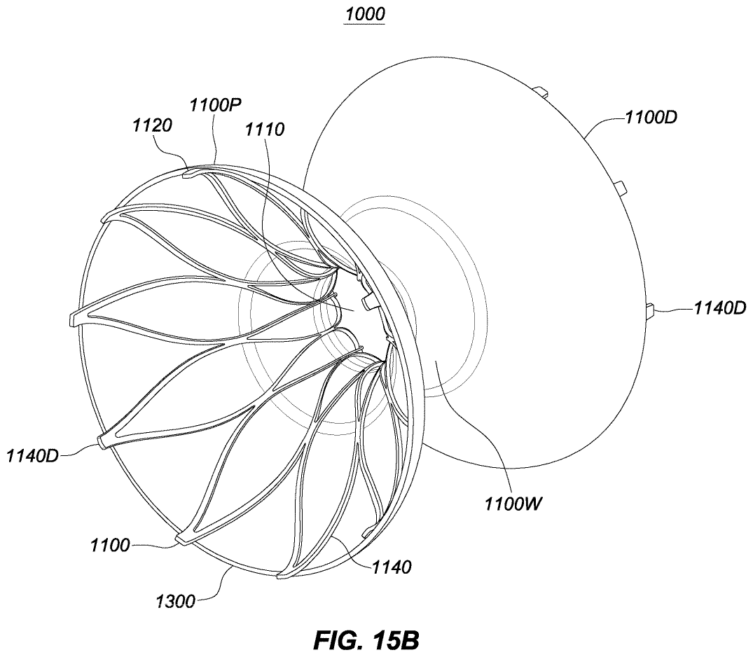

Additionally and/or alternatively, the radiopaque markers can be embedded in, or otherwise associated with, an optional cover member 1300 (shown in FIGS. 15A-B and 16A-B) of the transcatheter pulmonary flow reduction device 1000. By associating the radiopaque markers with the cover member 1300, the transcatheter pulmonary flow reduction device 1000 can maintain a smooth external periphery 1120 or outer profile while providing radiographic visibility. The transcatheter pulmonary flow reduction device 1000 alternatively can be coated with a radiopaque material to enhance visibility. The entire device frame 1100, for example, can be coated with the radiopaque material. In selected embodiments, the radiopaque material can be disposed on one or more of the proximal frame end region 1100P, the central waist region 1100W, the distal frame end region 1100D, the device retrieval system 1200 and/or one or more functional coupling components, such as the coupling device 1240, 1260, 1270, 1280 (shown in FIGS. 9A-B, 11A-B, 13A-B and 30A-B) of the device retrieval system 1200. The radiopaque material can be disposed on one or more device frame struts 1140 of the proximal frame end region 1100P, the central waist region 1100W and/or the distal frame end region 1100D and/or one or more device retrieval members 1210 of the device retrieval system 1200, without limitation.

Exemplary radiopaque coatings can include, but are not limited to, barium sulfate, bismuth subcarbonate and/or bismuth oxychloride that optionally can be incorporated into one or more polymer matrices and/or applied as a surface coating. In selected embodiments, the radiopaque coatings can be deposited onto the device frame 1100 and/or incorporated into the covering material of the cover member 1300 for improving visibility of the transcatheter pulmonary flow reduction device 1000 while retaining pliability and biocompatibility. The choice of radiopaque strategy can depend upon one or more selection criteria, such as a desired imaging clarity, an implantation depth, a covering design, at least one anatomical constraint and/or a need for precise localization of the transcatheter pulmonary flow reduction device 1000 during deployment and/or retrieval.

The transcatheter pulmonary flow reduction device 1000 can include an optional annular cover member 1300 as illustrated in FIGS. 15A-B. Stated somewhat differently, the cover member 1300 can be associated with the transcatheter pulmonary flow reduction device 1000, including the transcatheter pulmonary flow reduction device 1000 as shown and described herein with reference to FIGS. 2, 3A-B, 6A-B, 7A-B, 8A-B, 9A-B, 10A-B, 11A-B, 12A-B, 13A-B and/or 14A-B. In selected embodiments, at least a portion of the device frame 1100 can be covered, enclosed or otherwise encapsulated by the optional cover member 1300.

The cover member 1300 advantageously can be configured for restricting blood flow 122 (shown in FIGS. 4A-D and 5A-E) through the internal channel 1110 defined by the transcatheter pulmonary flow reduction device 1000 when deployed. Additionally and/or alternatively, the cover member 1300 can facilitate controlled blood flow 122, reduce tissue ingrowth, enhance sealing and/or modulate thrombotic behavior when the transcatheter pulmonary flow reduction device 1000 is deployed. The cover member 1300 can comprise any suitable covering material. The covering material preferably comprises a biocompatible cover material. Exemplary suitable covering materials can include, but are not limited to, expanded polytetrafluoroethylene (ePTFE), polyurethane-based materials, such as medical-grade urethane, and/or polyethylene terephthalate (PET).

The cover member 1300 can be applied to, or otherwise associated with, one or more selected portions of the transcatheter pulmonary flow reduction device 1000. As shown in FIGS. 15A-B, for example, the cover member 1300 can be disposed circumferentially around the external periphery 1120 of the transcatheter pulmonary flow reduction device 1000. The cover member 1300, in selected embodiments can be disposed circumferentially on an internal surface of the device frame 1100 and/or circumferentially on an external surface of the device frame 1100. The cover member 1300 can be affixed or otherwise coupled with the device frame 1100 in any suitable manner. Exemplary manners for coupling the cover member 1300 and the device frame 1100 can include suturing, laser bonding, heat bonding, adhesive bonding, welding, friction-fitting and/or encapsulation processes, without limitation. The cover member 1300 optionally can be applied as a single layer, multiple layers and/or selectively tensioned membranes to achieve desired flexibility, sealing and/or flow-control properties.

Additionally and/or alternatively, the cover member 1300 can envelop the entire device frame 1100, including the proximal frame end region 1100P, the central waist region 1100W and the distal frame end region 1100D. The cover member 1300 optionally can terminate distally to the distal strut end regions 1140D of the proximal frame end region 1100P and/or proximally to the distal strut end regions 1140D of the distal frame end region 1100D. Stated somewhat differently, the distal strut end regions 1140D of the proximal frame end region 1100P and/or the distal frame end region 1100D can extend beyond the cover member 1300 and otherwise be exposed. The exposed distal strut end regions 1140D thereby can be configured for engaging the vessel wall or internal lumen surface 124 (shown in FIG. 5A) of the pulmonary artery 120 (shown in FIG. 1) and otherwise providing mechanical anchoring for the transcatheter pulmonary flow reduction device 1000.

In selected embodiments, the cover member 1300 can extend to (or beyond) the distal strut end regions 1140D of the proximal frame end region 1100P and/or the distal frame end region 1100D such that the distal strut end regions 1140D can be fully encapsulated. The encapsulated distal strut end regions 1140D advantageously can help to reduce vessel trauma at the pulmonary artery 120 and/or inhibit endothelial ingrowth along the device frame struts 1140 or distal strut end regions 1140D of the device frame 1100. Although illustrated in FIGS. 15A-B as spanning an entire longitudinal length of the transcatheter pulmonary flow reduction device 1000 from the proximal frame end region 1100P to the distal frame end region 1100D, the cover member 1300 can extend distally from the proximal frame end region 1100P of the transcatheter pulmonary flow reduction device 1000 by any predetermined distance. The cover member 1300, in other words, can fully or partially span the longitudinal length of the transcatheter pulmonary flow reduction device 1000.

If comprising a self-expanding structure, such as a self-expanding Nitinol structure, for example, the device frame 1100 can be shape set in an hourglass-shape, wherein the internal channel 1110 at the central waist region 1100W is shape set with an internal diameter having a range between about three millimeters and five millimeters, without limitation. The device frame 1100 can be covered with the cover member 1300 that ultimately restricts flow through the internal channel 1110. The cover member 1300 located at the central waist region 1100W of the transcatheter pulmonary flow reduction device 1000 can constrain the internal channel 1110 at the central waist region 1100W. Stated somewhat differently, the cover member 1300 can restrict the internal diameter of the internal channel 1110 at the central waist region 1100W to the first dimension DW (shown in FIG. 5A). The first dimension DW, for example, can have a range between about one millimeter and two millimeters, without limitation.

Since the cover member 1300 is configured to expand, the internal diameter of the internal channel 1110 at the central waist region 1100W can be configured to increase, for example, by disposing a balloon catheter system or other expansion catheter system 3000 (shown in FIGS. 5A-E) within the internal channel 1110 at the central waist region 1100W. The implant expansion system 3120 of the expansion catheter system 3000 can be activated for radially re-expanding the internal channel 1110 at the waist region 1100W from the internal dimension DW to a second (or enlarged) internal dimension DWE (shown in FIG. 5E) that is greater than the internal dimension DW in the manner shown and described above with reference to FIGS. 5A-E. The internal channel 1110 of the central waist region 1100W thereby can be radially re-expanded from the internal dimension DW to the second internal dimension DWE (shown in FIG. 5E). As the implant expansion system 3120 expands, the cover member 1300 can deforms and thereby can allow the internal channel 1110 at the central waist region 1100W to expand out to its original shape set size such that the internal channel 1110 at the waist region 1100W can expand from the internal dimension DW to the second internal dimension DWE.

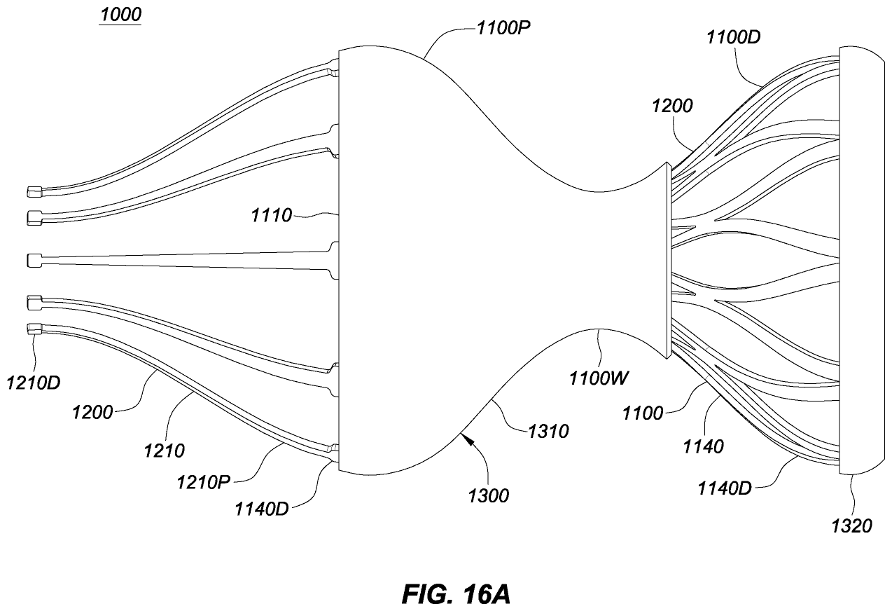

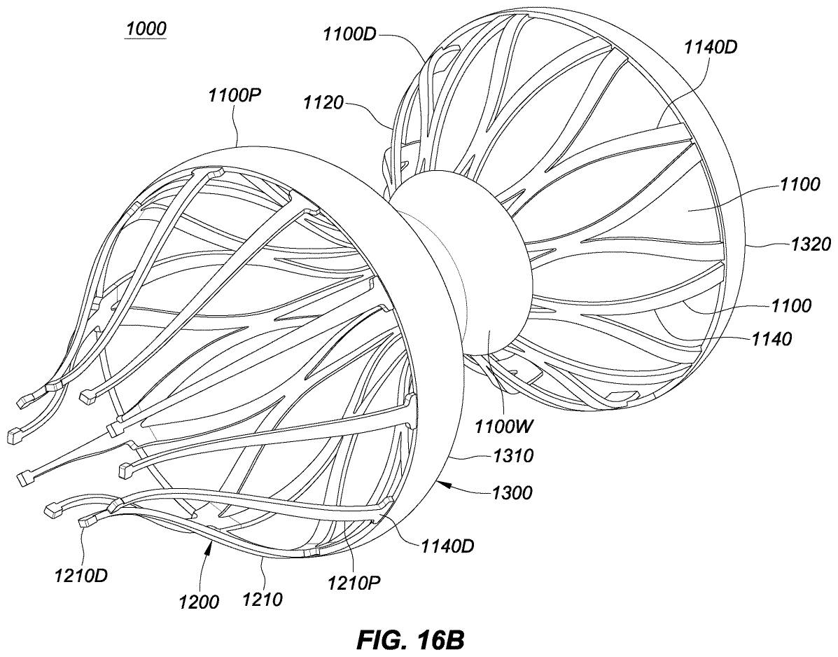

The transcatheter pulmonary flow reduction device 1000 optionally can include the device retrieval system 1200 and/or the annular cover member 1300 as shown in FIGS. 16A-B. Although the device retrieval system 1200 can comprise any of the device retrieval systems 1200 as disclosed herein, the transcatheter pulmonary flow reduction device 1000 of FIGS. 16A-B is shown as comprising the device retrieval system 1200 with a plurality of device retrieval members 1210 with respective distal retrieval member end regions 1210D extending proximally from the proximal frame end region 1100P of the device frame 1100 in the manner shown and described herein with reference to the device retrieval system 1200 of FIGS. 6A-B for purposes of illustration only.

The cover member 1300 can be configured for restricting blood flow 122 (shown in FIGS. 4A-D and 5A-E) through the internal channel 1110 defined by the transcatheter pulmonary flow reduction device 1000, when deployed. As shown in FIGS. 16A-B, the cover member 1300 can comprise a first annular cover member 1310 that can be disposed circumferentially around the external periphery 1120 of the transcatheter pulmonary flow reduction device 1000. The first cover member 1310, in selected embodiments can be disposed circumferentially on an internal surface of the device frame 1100 and/or circumferentially on an external surface of the device frame 1100. The first cover member 1310 can be provided in any suitable manner. The first annular cover member 1310, for example, can be provided in the manner shown and described about with reference to the cover member 1300 of FIGS. 15A-B.

In the manner set forth above with reference to the cover member 1300 of FIGS. 15A-B, the first cover member 1310 of FIGS. 16A-B can be applied to, or otherwise associated with, one or more selected portions of the transcatheter pulmonary flow reduction device 1000. The first cover member 1310, in selected embodiments, can be affixed or otherwise coupled with the device frame 1100 in any suitable manner. Exemplary manners for coupling the first cover member 1310 and the device frame 1100 can include suturing, laser bonding, heat bonding, adhesive bonding, welding, friction-fitting and/or encapsulation processes, without limitation. The first cover member 1310 optionally can be applied as a single layer, multiple layers and/or selectively tensioned membranes to achieve desired flexibility, sealing and/or flow-control properties.

The first cover member 1310 is shown in FIGS. 16A-B, for example, as being applied to, or otherwise associated with, the proximal frame end region 1100P and the central waist region 1100W of the device frame 1100. By associating the first cover member 1310 with the proximal frame end region 1100P, the transcatheter pulmonary flow reduction device 1000 can help to provide enhanced apposition and sealing against the vessel wall or internal lumen surface 124 (shown in FIG. 5A) of the pulmonary artery 120 (shown in FIG. 1) while reducing a potential for ingrowth at an anchoring location of the device frame 1100. The first cover member 1310, in selected embodiments may not extend to the distal frame end region 1100D of the device frame 1100, leaving the distal frame end region 1100D uncovered. The uncovered distal frame end region 1100D advantageously can permit blood present in one or more saddle regions 1150 (shown in FIG. 28) above and below the central waist region 1100W to move freely, reducing a risk of stasis and/or minimizing thrombosis. Alternatively, the first cover member 1310 optionally can extend distally from the proximal frame end region 1100P and the central waist region 1100W by any predetermined distance such that at least part of the distal frame end region 1100D can be associated with the first cover member 1310. The first cover member 1310, in other words, can fully or partially span the longitudinal length of the transcatheter pulmonary flow reduction device 1000.

In selected embodiment, the first cover member 1310 optionally can terminate distally to the distal strut end regions 1140D of the proximal frame end region 1100P. Stated somewhat differently, the distal strut end regions 1140D of the proximal frame end region 1100P can extend beyond the first cover member 1310 and otherwise be exposed. The exposed distal strut end regions 1140D thereby can be configured for engaging the vessel wall or internal lumen surface 124 (shown in FIG. 5A) of the pulmonary artery 120 (shown in FIG. 1) and otherwise providing mechanical anchoring for the transcatheter pulmonary flow reduction device 1000. The device retrieval members 1210 of the device retrieval system 1200 optionally can extend beyond the first cover member 1310 and likewise be exposed.

The first cover member 1310 alternatively can extend to (or beyond) the distal strut end regions 1140D of the proximal frame end region 1100P such that the distal strut end regions 1140D can be fully encapsulated. The encapsulated distal strut end regions 1140D advantageously can help to reduce vessel trauma at the pulmonary artery 120 and/or inhibit endothelial ingrowth along the device frame struts 1140 or distal strut end regions 1140D of the device frame 1100. In selected embodiments, the device retrieval members 1210 of the device retrieval system 1200 can be at least partially encapsulated by the first cover member 1310. The first cover member 1310, in other words, can be configured to extend proximally from the distal strut end regions 1140D of the proximal frame end region 1100P and to be associated with at least a portion of the device retrieval members 1210.

In the manner discussed in more detail with reference to the cover member 1300 of FIGS. 15A-B, the first cover member 1310 advantageously can be configured for restricting blood flow 122 (shown in FIGS. 4A-D and 5A-E) through the internal channel 1110 defined by the transcatheter pulmonary flow reduction device 1000 when deployed. The first cover member 1310 optionally can facilitate controlled blood flow 122, reduce tissue ingrowth, enhance sealing and/or modulate thrombotic behavior when the transcatheter pulmonary flow reduction device 1000 is deployed.

In selected configurations, the cover member 1300 optionally can include a second annular cover member 1320 as illustrated in FIGS. 16A-B. The second cover member 1320 can be provided in any suitable manner. The second cover member 1320, for example, can be provided in the manner shown and described about with reference to the cover member 1300 of FIGS. 15A-B. Like the first cover member 1310, the second cover member 1320 can comprise any suitable covering material. The covering material preferably comprises a biocompatible cover material. Exemplary suitable covering materials can include, but are not limited to, expanded polytetrafluoroethylene (ePTFE), polyurethane-based materials, such as medical-grade urethane, and/or polyethylene terephthalate (PET).

In the manner set forth above with reference to the cover member 1300 of FIGS. 15A-B, the second cover member 1320 can be applied to, or otherwise associated with, one or more selected portions of the transcatheter pulmonary flow reduction device 1000. As shown in FIGS. 16A-B, for example, the second cover member 1320 can be disposed circumferentially around the external periphery 1120 of the transcatheter pulmonary flow reduction device 1000. The second cover member 1320, in selected embodiments, can be disposed circumferentially on an internal surface of the device frame 1100 and/or circumferentially on an external surface of the device frame 1100. The second cover member 1320 can be affixed or otherwise coupled with the device frame 1100 in any suitable manner. Exemplary manners for coupling the second cover member 1320 and the device frame 1100 can include suturing, laser bonding, heat bonding, adhesive bonding, welding, friction-fitting and/or encapsulation processes, without limitation. The second cover member 1320 optionally can be applied as a single layer, multiple layers and/or selectively tensioned membranes to achieve desired flexibility, sealing and/or flow-control properties.

The second cover member 1320 can be applied to, or otherwise associated with, the external periphery 1120 of the distal frame end region 1100D of the device frame 1100. As shown in FIGS. 16A-B, for example, as being applied to, or otherwise associated with, the distal strut end regions 1140D of the distal frame end region 1100D. In selected embodiment, a portion of the distal strut end regions 1140D of the distal frame end region 1100D can extend beyond the second cover member 1320 and otherwise be exposed. The exposed distal strut end regions 1140D thereby can be configured for engaging the vessel wall or internal lumen surface 124 (shown in FIG. 5A) of the pulmonary artery 120 (shown in FIG. 1) and otherwise providing mechanical anchoring for the transcatheter pulmonary flow reduction device 1000. The second cover member 1320 alternatively can fully encapsulate the distal strut end regions 1140D of the distal frame end region 1100D. By the distal strut end regions 1140D of the distal frame end region 1100D, the second cover member 1320 advantageously can help to help to prevent ingrowth at the distal strut end regions 1140D while still allowing open-cell flow pathways through the remainder of the distal frame end region 1100D.

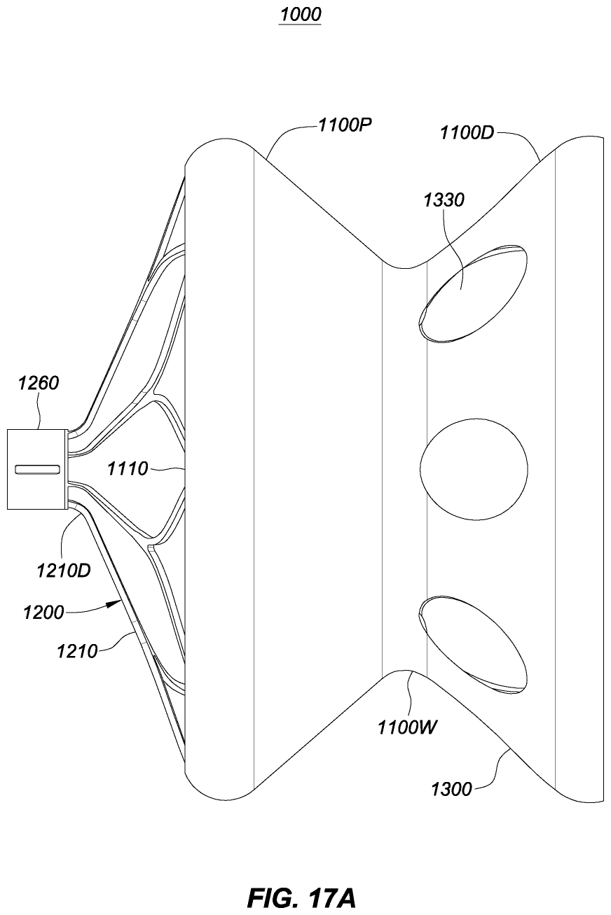

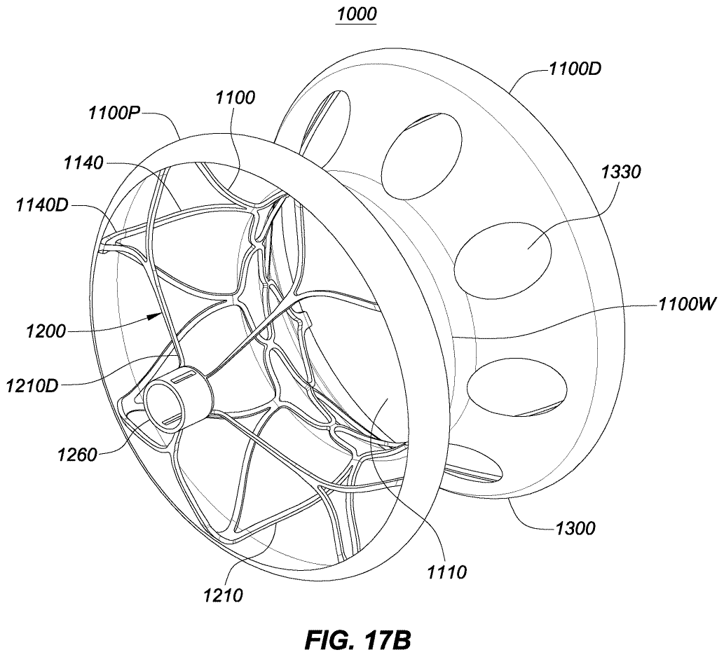

In the manner discussed above, the transcatheter pulmonary flow reduction device 1000 optionally can include the device retrieval system 1200 and/or the annular cover member 1300. The transcatheter pulmonary flow reduction device 1000 of FIGS. 17A-B, for example, is shown as including the device retrieval system 1200 and the annular cover member 1300. Although the device retrieval system 1200 can comprise any of the device retrieval systems 1200 as disclosed herein, the transcatheter pulmonary flow reduction device 1000 of FIGS. 17A-B is shown as comprising the device retrieval system 1200 with a plurality of device retrieval members 1210 with respective distal retrieval member end regions 1210D extending proximally from the proximal frame end region 1100P of the device frame 1100 and converging at the coupling device 1260 in the manner shown and described herein with reference to the device retrieval system 1200 of FIGS. 11A-B for purposes of illustration only.

The cover member 1300 can be configured for restricting blood flow 122 (shown in FIGS. 4A-D and 5A-E) through the internal channel 1110 defined by the transcatheter pulmonary flow reduction device 1000, when deployed, and can be disposed circumferentially around the external periphery 1120 of the transcatheter pulmonary flow reduction device 1000. In selected embodiments, the cover member 1300 can be disposed circumferentially on an internal surface of the device frame 1100 and/or circumferentially on an external surface of the device frame 1100. The cover member 1300 can be provided in any suitable manner. The first annular cover member 1310, for example, can be provided in the manner shown and described about with reference to the cover member 1300 of FIGS. 15A-B.

In the manner set forth above with reference to the cover member 1300 of FIGS. 15A-B, the cover member 1300 of FIGS. 17A-B can be applied to, or otherwise associated with, one or more selected portions of the transcatheter pulmonary flow reduction device 1000. The cover member 1300, in selected embodiments, can be affixed or otherwise coupled with the device frame 1100 in any suitable manner. Exemplary manners for coupling the cover member 1300 and the device frame 1100 can include suturing, laser bonding, heat bonding, adhesive bonding, welding, friction-fitting and/or encapsulation processes, without limitation. The cover member 1300 optionally can be applied as a single layer, multiple layers and/or selectively tensioned membranes to achieve desired flexibility, sealing and/or flow-control properties.

The cover member 1300 is shown in FIGS. 17A-B, for example, as being applied to, or otherwise associated with, the proximal frame end region 1100P, the central waist region 1100W and the distal frame end region 1100D of the device frame 1100. Stated somewhat differently, the stent frame 1100 can be entirely encompassed by the cover member 1300 in selected embodiments. The cover member 1300 optionally can define at least one window, perforation or other cover member opening 1330. As illustrated in FIGS. 17A-B, one or more of the cover member openings 1330 can be defined in the cover member 1300 adjacent to the distal frame end region 1100D of the device frame 1100.

The cover member openings 1330 can be formed in the cover member 1300 in any suitable matter. Exemplary manners for forming the cover member openings 1330 in the cover member 1300 can include, but are not limited to, laser machining, mechanical punching, thermal forming or selective masking during a process for applying the cover member 1300 to the device frame 1100. The cover member openings 1330 advantageously can allow blood movement into and out of a saddle region 1150 (shown in FIG. 28) adjacent to the central waist region 1100W, thereby reducing a likelihood of thrombosis. Additionally and/or alternatively, by covering most of the distal frame end region 1100D of the device frame 1100, tissue ingrowth along the device frame 1100 can be limited, improving retrievability and/or maintaining the adjustment functionality of the central waist region 1100W of the device frame 1100.

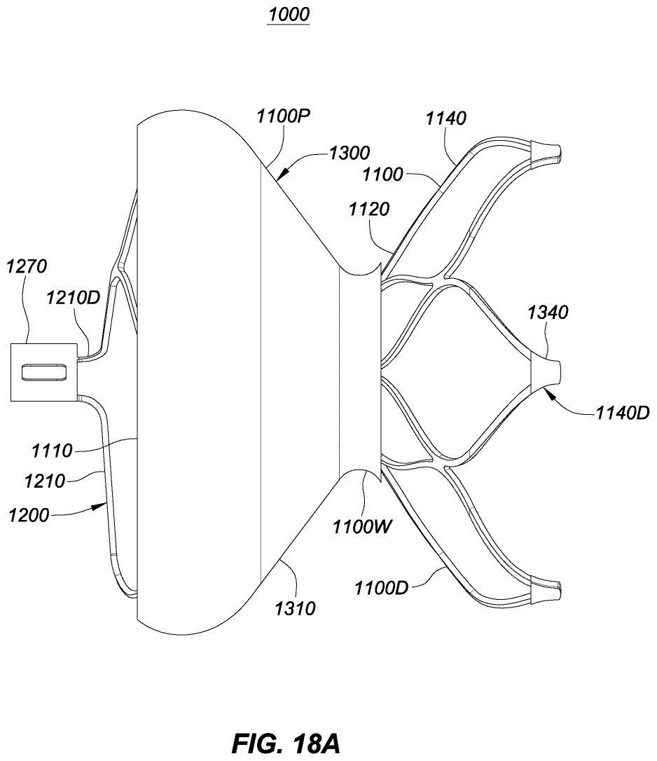

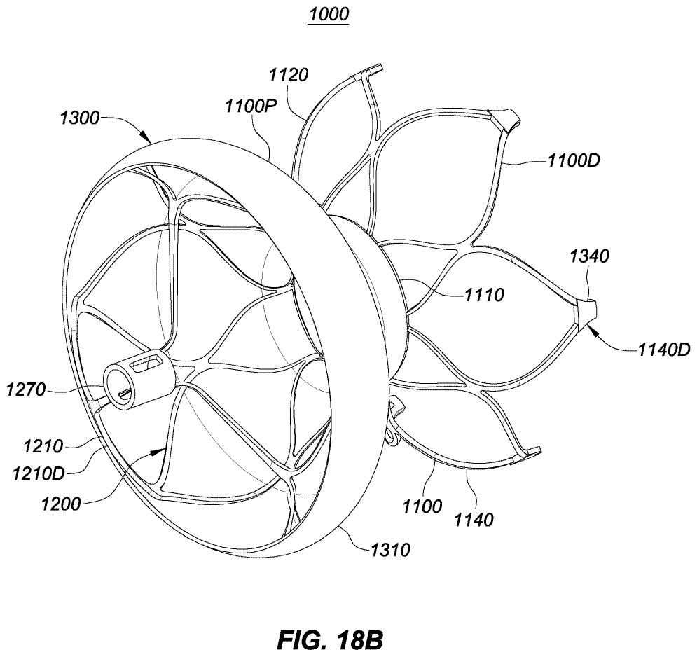

The transcatheter pulmonary flow reduction device 1000 optionally can include the device retrieval system 1200 and/or the annular cover member 1300 in the manner discussed above. Turning to FIGS. 18A-B, for example, the transcatheter pulmonary flow reduction device 1000 can include the device retrieval system 1200 and the annular cover member 1300. Although the device retrieval system 1200 can comprise any of the device retrieval systems 1200 as disclosed herein, the transcatheter pulmonary flow reduction device 1000 of FIGS. 18A-B is shown as comprising the device retrieval system 1200 with a plurality of device retrieval members 1210 with respective distal retrieval member end regions 1210D extending proximally from the proximal frame end region 1100P of the device frame 1100 and converging at the coupling device 1270 in the manner shown and described herein with reference to the device retrieval system 1200 of FIGS. 14A-B for purposes of illustration only.

The cover member 1300 can be configured for restricting blood flow 122 (shown in FIGS. 4A-D and 5A-E) through the internal channel 1110 defined by the transcatheter pulmonary flow reduction device 1000, when deployed. As shown in FIGS. 18A-B, the cover member 1300 can comprise a first annular cover member 1310 that can be disposed circumferentially around the external periphery 1120 of the transcatheter pulmonary flow reduction device 1000. The first cover member 1310, in selected embodiments can be disposed circumferentially on an internal surface of the device frame 1100 and/or circumferentially on an external surface of the device frame 1100. The first cover member 1310 can be provided in any suitable manner. The first annular cover member 1310, for example, can be provided in the manner shown and described about with reference to the cover member 1300 of FIGS. 15A-B.

In the manner set forth above with reference to the cover member 1300 of FIGS. 15A-B, the first cover member 1310 of FIGS. 18A-B can be applied to, or otherwise associated with, one or more selected portions of the transcatheter pulmonary flow reduction device 1000. The first cover member 1310, in selected embodiments, can be affixed or otherwise coupled with the device frame 1100 in any suitable manner. Exemplary manners for coupling the first cover member 1310 and the device frame 1100 can include suturing, laser bonding, heat bonding, adhesive bonding, welding, friction-fitting and/or encapsulation processes, without limitation. The first cover member 1310 optionally can be applied as a single layer, multiple layers and/or selectively tensioned membranes to achieve desired flexibility, sealing and/or flow-control properties.

The first cover member 1310 is shown in FIGS. 18A-B, for example, as being applied to, or otherwise associated with, the proximal frame end region 1100P and the central waist region 1100W of the device frame 1100. By associating the first cover member 1310 with the proximal frame end region 1100P, the transcatheter pulmonary flow reduction device 1000 can help to provide enhanced apposition and sealing against the vessel wall or internal lumen surface 124 (shown in FIG. 5A) of the pulmonary artery 120 (shown in FIG. 1) while reducing a potential for ingrowth at an anchoring location of the device frame 1100. The first cover member 1310, in other words, can help to ensure effective sealing in regions of the device frame 1100 that can be critical for maintaining a controlled reduction in blood flow 122 (shown in FIGS. 4A-D and 5A-E) through the internal channel 1110 defined by the transcatheter pulmonary flow reduction device 1000, when deployed.

The first cover member 1310, in selected embodiments may not extend to the distal frame end region 1100D of the device frame 1100, leaving the distal frame end region 1100D uncovered. The uncovered distal frame end region 1100D advantageously can permit blood present in one or more saddle regions 1150 (shown in FIG. 28) above and below the central waist region 1100W to move freely, reducing a risk of stasis and/or minimizing thrombosis. Stated somewhat differently, the uncovered distal frame end region 1100D can promote blood flow 122 across the saddle regions 1150 and/or reduce formation of blood clots. Alternatively, the first cover member 1310 can extend distally from the proximal frame end region 1100P and the central waist region 1100W by any predetermined distance such that at least part of the distal frame end region 1100D can be associated with the first cover member 1310. The first cover member 1310, in other words, can fully or partially span the longitudinal length of the transcatheter pulmonary flow reduction device 1000.

In selected embodiment, the first cover member 1310 optionally can terminate distally to the distal strut end regions 1140D of the proximal frame end region 1100P. Stated somewhat differently, the distal strut end regions 1140D of the proximal frame end region 1100P can extend beyond the first cover member 1310 and otherwise be exposed. The exposed distal strut end regions 1140D thereby can be configured for engaging the vessel wall or internal lumen surface 124 (shown in FIG. 5A) of the pulmonary artery 120 (shown in FIG. 1) and otherwise providing mechanical anchoring for the transcatheter pulmonary flow reduction device 1000. The device retrieval members 1210 of the device retrieval system 1200 optionally can extend beyond the first cover member 1310 and likewise be exposed.

The first cover member 1310 alternatively can extend to (or beyond) the distal strut end regions 1140D of the proximal frame end region 1100P such that the distal strut end regions 1140D can be fully encapsulated. The encapsulated distal strut end regions 1140D advantageously can help to reduce vessel trauma at the pulmonary artery 120 and/or inhibit endothelial ingrowth along the device frame struts 1140 or distal strut end regions 1140D of the device frame 1100. In selected embodiments, the device retrieval members 1210 of the device retrieval system 1200 can be at least partially encapsulated by the first cover member 1310. The first cover member 1310, in other words, can be configured to extend proximally from the distal strut end regions 1140D of the proximal frame end region 1100P and to be associated with at least a portion of the device retrieval members 1210.

In the manner discussed in more detail with reference to the cover member 1300 of FIGS. 15A-B, the first cover member 1310 advantageously can be configured for restricting blood flow 122 (shown in FIGS. 4A-D and 5A-E) through the internal channel 1110 defined by the transcatheter pulmonary flow reduction device 1000 when deployed. The first cover member 1310 optionally can facilitate controlled blood flow 122, reduce tissue ingrowth, enhance sealing and/or modulate thrombotic behavior when the transcatheter pulmonary flow reduction device 1000 is deployed.

In selected configurations, the cover member 1300 optionally can include a second annular cover member 1340 as illustrated in FIGS. 18A-B. The second cover member 1340 can be provided in any suitable manner. The second cover member 1340, for example, can be provided in the manner shown and described about with reference to the cover member 1300 of FIGS. 15A-B. Like the first cover member 1310, the second cover member 1340 can comprise any suitable covering material. The covering material preferably comprises a biocompatible cover material. Exemplary suitable covering materials can include, but are not limited to, expanded polytetrafluoroethylene (ePTFE), polyurethane-based materials, such as medical-grade urethane, and/or polyethylene terephthalate (PET).

In the manner set forth above with reference to the cover member 1300 of FIGS. 15A-B, the second cover member 1340 can be applied to, or otherwise associated with, one or more selected portions of the transcatheter pulmonary flow reduction device 1000. As shown in FIGS. 18A-B, for example, a plurality of the second cover members 1340 can be disposed on respective distal strut end regions 1140D of the distal frame end region 1100D. The second cover members 1340, in other words, can provide encapsulation patches or localized coverings for the distal strut end regions 1140D. By disposing the second cover members 1340 on the distal strut end regions 1140D of the distal frame end region 1100D, a likelihood of tissue ingrowth at the distal strut end regions 1140D can be mitigated. Additionally and/or alternatively, the uncovered distal frame end region 1100D likewise can facilitate ongoing blood flow exchange around the central waist region 1100W.

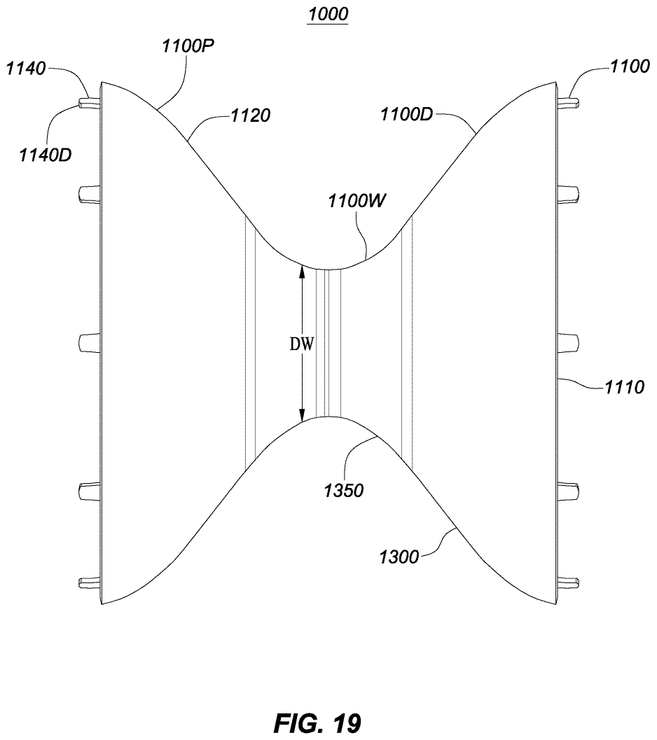

In selected embodiments, the cover member 1300 can be configured to restrict the central waist region 1100W of the transcatheter pulmonary flow reduction device 1000. The cover member 1300, in other words, can restrict the central waist region 1100W of the device frame 1100, when deployed. Turning to FIG. 19, for example, the transcatheter pulmonary flow reduction device 1000 is shown as including the optional cover member 1300. The cover member 1300 can be provided in the manner set forth herein with reference to the cover member 1300 of FIGS. 15A-B, 16A-B, 17A-B and 18A-B.

The transcatheter pulmonary flow reduction device 1000 is shown as comprising an hourglass-shaped device frame 1100 with a central waist region 1100W disposed between proximal and distal frame end regions 1100P, 1100D and defining an internal channel 1110 in the manner discussed in more detail above with reference to the device frame 1100, including the device frame 1100 as shown and described herein with reference to FIGS. 2, 3A-B, 6A-B, 7A-B, 8A-B, 9A-B, 10A-B, 11A-B, 12A-B, 13A-B and/or 14A-B. The external periphery 1120 of the proximal frame end region 1100P and the external periphery 1120 of the central waist region 1100W can define a first frame taper angle; whereas, the external periphery 1120 of the distal frame end region 1100F and the external periphery 1120 of the central waist region 1100W can define a second frame taper angle. The first and second frame taper angles can comprise any suitable angle, such as any predetermined angle within a taper angle range between twenty degrees and eighty degrees, or within any angle subrange of the taper angle range, without limitation. Although preferably comprising uniform angles, the first and second frame taper angles can be different in selected embodiments and/or in selected applications.

In selected embodiments, the cover member 1300 can extend to (or beyond) the distal strut end regions 1140D of the proximal frame end region 1100P and/or the distal frame end region 1100D such that the distal strut end regions 1140D can be fully encapsulated. The encapsulated distal strut end regions 1140D advantageously can help to reduce vessel trauma at the pulmonary artery 120 and/or inhibit endothelial ingrowth along the device frame struts 1140 or distal strut end regions 1140D of the device frame 1100. Although illustrated in FIGS. 15A-B as spanning an entire longitudinal length of the transcatheter pulmonary flow reduction device 1000 from the proximal frame end region 1100P to the distal frame end region 1100D, the cover member 1300 can extend distally from the proximal frame end region 1100P of the transcatheter pulmonary flow reduction device 1000 by any predetermined distance. The cover member 1300, in other words, can fully or partially span the longitudinal length of the transcatheter pulmonary flow reduction device 1000.

The cover member 1300 is illustrated in FIG. 19 as including a central cover region 1350 that is disposed circumferentially around the external periphery 1120 of the central waist region 1100W of the transcatheter pulmonary flow reduction device 1000. Stated somewhat differently, the central cover region 1350 of the cover member 1300 can be associated with, and/or cooperate with, the central waist region 1100W of the device frame 1100. The central cover region 1350 of the cover member 1300 can be configured to restrict or otherwise constrict the central waist region 1100W of the device frame 1100, when deployed. Thereby, the central cover region 1350 of the cover member 1300 can enable the internal channel 1110 of the central waist region 1100W to achieve and/or maintain the internal dimension DW when the transcatheter pulmonary flow reduction device 1000 is in the first stable expanded state. In selected embodiments, the cover member 1300 can comprise only the central cover region 1350 being disposed circumferentially around the external periphery 1120 of the central waist region 1100W.

After the initial deployment, the device frame 1100 subsequently can be re-expanded from the first stable expanded state to the second stable expanded state. The device frame 1100, for example, can be re-expanded from the first stable expanded state to the second stable expanded state via the expansion catheter system 3000 in the manner shown and described with reference to FIGS. 5A-E. The internal channel 1110 of the central waist region 1100W thereby can be radially re-expanded from the internal dimension DW to the second internal dimension DWE (shown in FIG. 5E) after deployment of the transcatheter pulmonary flow reduction device 1000.

In selected embodiments, the central cover region 1350 of the cover member 1300 can plastically deform, during re-expansion. The central cover region 1350 thereby can permit incremental expansion of the internal channel 1110 of the central waist region 1100W. If the cover member 1300 comprises one or more layers, for example, the layers of the central cover region 1350 advantageously can help to enhance control over the re-expansion of the internal channel 1110 of the central waist region 1100W and otherwise enable fine-tuned adjustments of flow reduction. The central cover region 1350 thereby can allow for higher-resolution flow control while maintaining consistent structural support around the central waist region 1100W of the device frame 1100. Although shown and described with reference to FIG. 19 as being formed from the cover material and otherwise being integrated with the cover member 1300, the central cover region 1350 of the cover member 1300 can be provided in any suitable manner. In selected embodiments, the cover member 1300 can be formed or otherwise provided from a polymer or cloth cover material; whereas, the central cover region 1350 can be formed or otherwise provided from an expandable polymer material, without limitation.

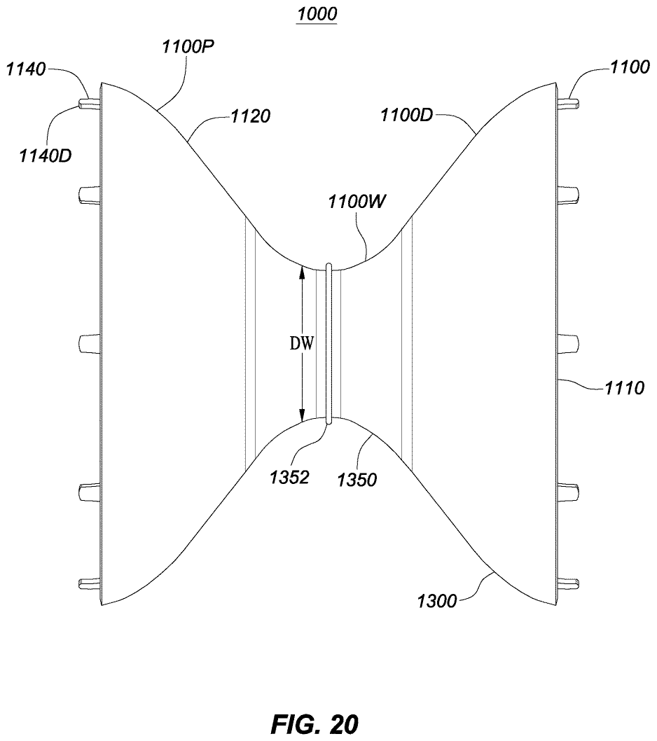

The central cover region 1350, in selected embodiments, can be separate from the cover member 1300. Turning to FIG. 20, for example, the transcatheter pulmonary flow reduction device 1000 is illustrated as including the optional cover member 1300. The cover member 1300 can be provided in the manner set forth herein with reference to the cover member 1300 of FIGS. 15A-B, 16A-B, 17A-B and 18A-B. The cover member 1300 can include the central cover region 1350, which is shown in FIG. 20 as comprising at least one loop member 1352 of suture, wire, string or other suitable cover loop material. The cover loop member 1352 can define an internal size, shape, diameter, cross-section or other dimension for receiving the cover member 1300 disposed around the device frame 1100.

The transcatheter pulmonary flow reduction device 1000 is shown as comprising an hourglass-shaped device frame 1100 with a central waist region 1100W disposed between proximal and distal frame end regions 1100P, 1100D and defining an internal channel 1110 in the manner discussed in more detail above with reference to the device frame 1100, including the device frame 1100 as shown and described herein with reference to FIGS. 2, 3A-B, 6A-B, 7A-B, 8A-B, 9A-B, 10A-B, 11A-B, 12A-B, 13A-B and/or 14A-B. The external periphery 1120 of the proximal frame end region 1100P and the external periphery 1120 of the central waist region 1100W can define a first frame taper angle; whereas, the external periphery 1120 of the distal frame end region 1100F and the external periphery 1120 of the central waist region 1100W can define a second frame taper angle. The first and second frame taper angles can comprise any suitable angle, such as any predetermined angle within a taper angle range between twenty degrees and eighty degrees, or within any angle subrange of the taper angle range, without limitation. Although preferably comprising uniform angles, the first and second frame taper angles can be different in selected embodiments and/or in selected applications.

In selected embodiments, the cover member 1300 can extend to (or beyond) the distal strut end regions 1140D of the proximal frame end region 1100P and/or the distal frame end region 1100D such that the distal strut end regions 1140D can be fully encapsulated. The encapsulated distal strut end regions 1140D advantageously can help to reduce vessel trauma at the pulmonary artery 120 and/or inhibit endothelial ingrowth along the device frame struts 1140 or distal strut end regions 1140D of the device frame 1100. Although illustrated in FIGS. 15A-B as spanning an entire longitudinal length of the transcatheter pulmonary flow reduction device 1000 from the proximal frame end region 1100P to the distal frame end region 1100D, the cover member 1300 can extend distally from the proximal frame end region 1100P of the transcatheter pulmonary flow reduction device 1000 by any predetermined distance. The cover member 1300, in other words, can fully or partially span the longitudinal length of the transcatheter pulmonary flow reduction device 1000.

As shown in FIG. 20, the cover loop member 1352 can be circumferentially disposed around the external periphery 1120 of the central waist region 1100W of the transcatheter pulmonary flow reduction device 1000. Stated somewhat differently, the circumferential cover loop member 1352 of the cover member 1300 can be associated with, and/or cooperate with, the central waist region 1100W of the device frame 1100. In selected embodiments, the circumferential cover loop member 1352 can be disposed around an external periphery of the cover member 1300 adjacent to the central waist region 1100W of the device frame 1100. The circumferential cover loop member 1352 alternatively can be disposed directly around the external periphery 1120 of the central waist region 1100W of the device frame 1100. The cover loop member 1352 can be cinched or otherwise configured to restrict or otherwise constrict the central waist region 1100W of the device frame 1100, when deployed. Thereby, the central cover region 1350 of the cover member 1300 can enable the internal channel 1110 of the central waist region 1100W to achieve and/or maintain the internal dimension DW when the transcatheter pulmonary flow reduction device 1000 is in the first stable expanded state. An external size, shape, diameter, cross-section or other dimension of the central waist region 1100W in the first stable expanded state, in other words, can be limited or otherwise constrained by the internal dimension of the cover loop member 1352.

After the initial deployment, the device frame 1100 subsequently can be re-expanded from the first stable expanded state to the second stable expanded state. The device frame 1100, for example, can be radially re-expanded from the first stable expanded state to the second stable expanded state via the expansion catheter system 3000 in the manner shown and described with reference to FIGS. 5A-E. During re-expansion of the device frame 1100, the external dimension of the central waist region 1100W can increase. The central waist region 1100W can break or otherwise open the cover loop member 1352 when the increased external dimension of the central waist region 1100W exceeds the internal dimension of the cover loop member 1352. The external dimension of the central waist region 1100W in the second stable expanded state thereby can be no longer constrained by the internal dimension of the cover loop member 1352. The internal channel 1110 of the central waist region 1100W thus can be radially re-expanded from the internal dimension DW to the second internal dimension DWE (shown in FIG. 5E) after deployment of the transcatheter pulmonary flow reduction device 1000.

In selected embodiments, the central cover region 1350 can include a plurality of the cover loop members 1352, each comprising a suitable cover loop material. The cover loop material can be uniform and/or different among the cover loop members 1352. The cover loop members 1352 can define respective internal sizes, shapes, diameters, cross-sections or other dimensions for receiving the cover member 1300 disposed around the device frame 1100, wherein the internal dimensions of the cover loop members 1352 can be the same and/or different. The cover loop members 1352 preferably define incrementally-increasing internal dimensions.

During re-expansion of the device frame 1100, the external dimension of the central waist region 1100W can increase, and the central waist region 1100W can break or otherwise open the cover loop members 1352 with internal dimensions that are less than the increased external dimension of the central waist region 1100W. The cover loop members 1352 with internal dimensions that are greater than or equal to the increased external dimension of the central waist region 1100W can remain unbroken and otherwise intact. The external dimension of the central waist region 1100W in the second stable expanded state thereby can be constrained by the internal dimension of the smallest intact cover loop member 1352. Thereby, the central cover region 1350 advantageously can provide controlled modulation of flow through the internal channel 1110 of the transcatheter pulmonary flow reduction device 1000 in a manner that supports straightforward adjustment of flow reduction and maintains a compact delivery (or implantation) profile prior to expansion.

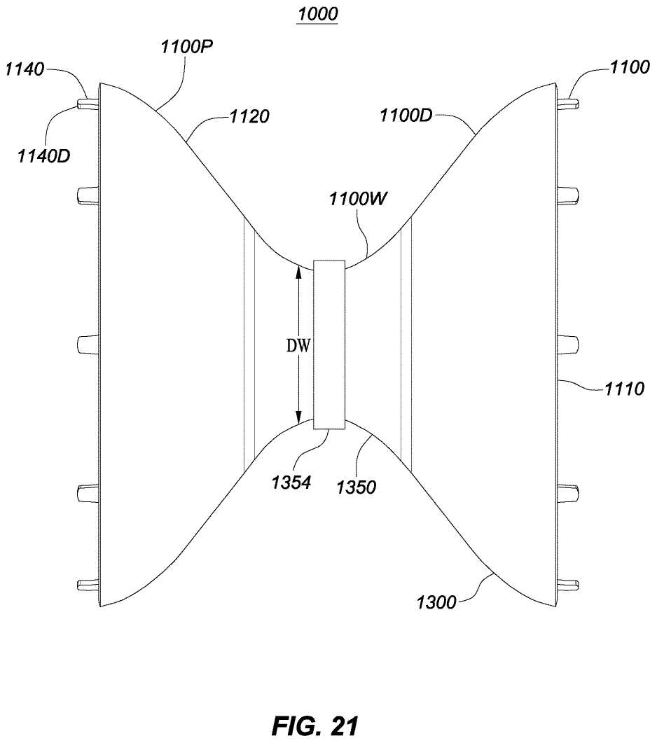

Additionally and/or alternatively, the central cover region 1350 can be provided as an expandable band member. Turning to FIG. 21, for example, the transcatheter pulmonary flow reduction device 1000 is illustrated as including the optional cover member 1300. The cover member 1300 can be provided in the manner set forth herein with reference to the cover member 1300 of FIGS. 15A-B, 16A-B, 17A-B and 18A-B. The cover member 1300 can include the central cover region 1350, which is shown in FIG. 21 as comprising at least one expandable band member 1354 that can be formed or otherwise provided from a suitable expandable material, such as an expandable polymer material. The expandable band member 1354 can define an internal size, shape, diameter, cross-section or other dimension for receiving the cover member 1300 disposed around the device frame 1100.

The transcatheter pulmonary flow reduction device 1000 is shown as comprising an hourglass-shaped device frame 1100 with a central waist region 1100W disposed between proximal and distal frame end regions 1100P, 1100D and defining an internal channel 1110 in the manner discussed in more detail above with reference to the device frame 1100, including the device frame 1100 as shown and described herein with reference to FIGS. 2, 3A-B, 6A-B, 7A-B, 8A-B, 9A-B, 10A-B, 11A-B, 12A-B, 13A-B and/or 14A-B. The external periphery 1120 of the proximal frame end region 1100P and the external periphery 1120 of the central waist region 1100W can define a first frame taper angle; whereas, the external periphery 1120 of the distal frame end region 1100F and the external periphery 1120 of the central waist region 1100W can define a second frame taper angle. The first and second frame taper angles can comprise any suitable angle, such as any predetermined angle within a taper angle range between twenty degrees and eighty degrees, or within any angle subrange of the taper angle range, without limitation. Although preferably comprising uniform angles, the first and second frame taper angles can be different in selected embodiments and/or in selected applications.

In selected embodiments, the cover member 1300 can extend to (or beyond) the distal strut end regions 1140D of the proximal frame end region 1100P and/or the distal frame end region 1100D such that the distal strut end regions 1140D can be fully encapsulated. The encapsulated distal strut end regions 1140D advantageously can help to reduce vessel trauma at the pulmonary artery 120 and/or inhibit endothelial ingrowth along the device frame struts 1140 or distal strut end regions 1140D of the device frame 1100. Although illustrated in FIGS. 15A-B as spanning an entire longitudinal length of the transcatheter pulmonary flow reduction device 1000 from the proximal frame end region 1100P to the distal frame end region 1100D, the cover member 1300 can extend distally from the proximal frame end region 1100P of the transcatheter pulmonary flow reduction device 1000 by any predetermined distance. The cover member 1300, in other words, can fully or partially span the longitudinal length of the transcatheter pulmonary flow reduction device 1000.

As shown in FIG. 21, the expandable band member 1354 can be circumferentially disposed around the external periphery 1120 of the central waist region 1100W of the transcatheter pulmonary flow reduction device 1000. Stated somewhat differently, the circumferential expandable band member 1354 of the cover member 1300 can be associated with, and/or cooperate with, the central waist region 1100W of the device frame 1100. In selected embodiments, the circumferential expandable band member 1354 can be disposed around an external periphery of the cover member 1300 adjacent to the central waist region 1100W of the device frame 1100. The circumferential expandable band member 1354 alternatively can be disposed directly around the external periphery 1120 of the central waist region 1100W of the device frame 1100. The expandable band member 1354 can be configured to restrict or otherwise constrict the central waist region 1100W of the device frame 1100, when deployed. Thereby, the central cover region 1350 of the cover member 1300 can enable the internal channel 1110 of the central waist region 1100W to achieve and/or maintain the internal dimension DW when the transcatheter pulmonary flow reduction device 1000 is in the first stable expanded state. An external size, shape, diameter, cross-section or other dimension of the central waist region 1100W in the first stable expanded state, in other words, can be limited or otherwise constrained by the internal dimension of the expandable band member 1354.

After the initial deployment, the device frame 1100 subsequently can be re-expanded from the first stable expanded state to the second stable expanded state. The device frame 1100, for example, can be radially re-expanded from the first stable expanded state to the second stable expanded state via the expansion catheter system 3000 in the manner shown and described with reference to FIGS. 5A-E. During re-expansion of the device frame 1100, the expandable band member 1354 can plastically deform, permitting the external dimension of the central waist region 1100W to (gradually) increase. The external dimension of the central waist region 1100W in the second stable expanded state thereby can be constrained by the expanded internal dimension of the deformed expandable band member 1354. The internal channel 1110 of the central waist region 1100W thus can be radially re-expanded from the internal dimension DW to the second internal dimension DWE (shown in FIG. 5E) after deployment of the transcatheter pulmonary flow reduction device 1000.

The expandable band member 1354 advantageously can permit controlled adjustment of the external dimension of the central waist region 1100W and/or can maintain structural integrity of the transcatheter pulmonary flow reduction device 1000 before, during and/or after re-expansion. In selected embodiments, the central cover region 1350 can comprise a single band member 1354 provided in a layered configuration and/or a plurality of expandable band members 1354. The single band member 1354 in the layered configuration and/or the plurality of expandable band members 1354 advantageously can support progressive tuning of the flow reduction through the internal channel 1110 of the transcatheter pulmonary flow reduction device 1000.

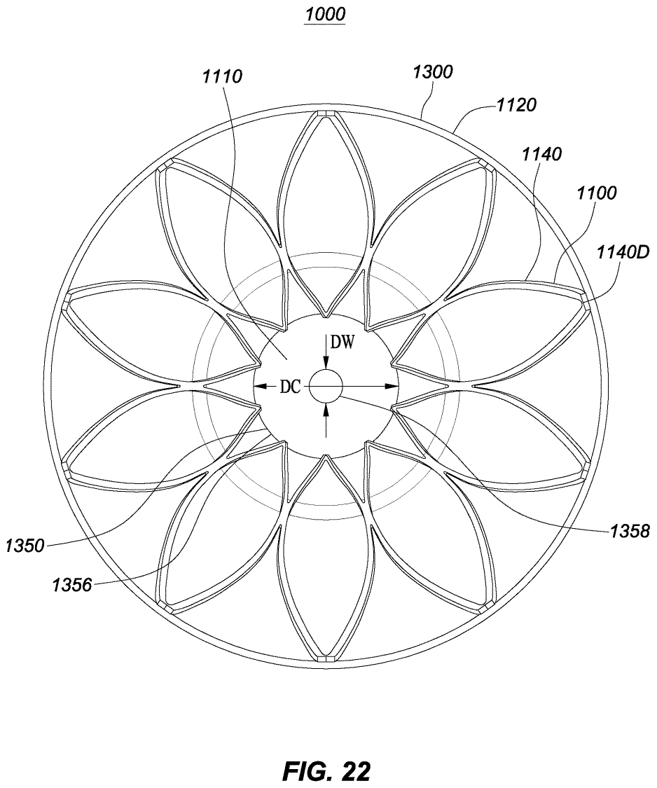

The central cover region 1350 optionally can be provided as at least one deformable annular septum member 1356 as illustrated in FIG. 22. Turning to FIG. 22, for example, the transcatheter pulmonary flow reduction device 1000 is illustrated as including the optional cover member 1300. The cover member 1300 can be provided in the manner set forth herein with reference to the cover member 1300 of FIGS. 15A-B, 16A-B, 17A-B and 18A-B. The cover member 1300 can include the central cover region 1350, which is shown in FIG. 22 as comprising at least one deformable septum member 1356 that can be formed or otherwise provided from a suitable septum member material, such as a deformable polymer material, without limitation.

The transcatheter pulmonary flow reduction device 1000 is shown as comprising an hourglass-shaped device frame 1100 with a central waist region 1100W disposed between proximal and distal frame end regions 1100P, 1100D and defining an internal channel 1110 in the manner discussed in more detail above with reference to the device frame 1100, including the device frame 1100 as shown and described herein with reference to FIGS. 2, 3A-B, 6A-B, 7A-B, 8A-B, 9A-B, 10A-B, 11A-B, 12A-B, 13A-B and/or 14A-B. The external periphery 1120 of the proximal frame end region 1100P and the external periphery 1120 of the central waist region 1100W can define a first frame taper angle; whereas, the external periphery 1120 of the distal frame end region 1100F and the external periphery 1120 of the central waist region 1100W can define a second frame taper angle. The first and second frame taper angles can comprise any suitable angle, such as any predetermined angle within a taper angle range between twenty degrees and eighty degrees, or within any angle subrange of the taper angle range, without limitation. Although preferably comprising uniform angles, the first and second frame taper angles can be different in selected embodiments and/or in selected applications.

In certain embodiments, pulmonary flow restriction provided by the transcatheter pulmonary flow reduction device 1000 may be substantially reduced and/or eliminated by placement of an additional expandable implant (not shown) within the internal channel 1110 at the central waist region 1100W. For example, a balloon-expandable metallic stent (not shown), such as a cobalt-chromium stent, may be advanced into the internal channel 1110 at the central waist region 1100W and expanded to a diameter corresponding to the enlarged proximal and distal frame end regions 1100P, 1100D. Expansion of the internal stent can force the central waist region 1100W to fully open, thereby restoring the effective lumen diameter and allowing pulmonary blood flow to return to a substantially unrestricted or pre-implant level. This approach advantageously can enable functional deactivation of the flow-restrictive central waist region 1100W without requiring retrieval or removal of the transcatheter pulmonary flow reduction device 1000 and may be particularly advantageous in staged therapies or clinical scenarios where permanent anchoring of the transcatheter pulmonary flow reduction device 1000 is desired but flow restriction is no longer required.

In certain embodiments, the internal channel 1110 at the central waist region 1100W can be configured to be selectively enlarged after implantation and/or deployment by the use of an appropriately-sized balloon catheter introduced through the internal channel 1110 of the transcatheter pulmonary flow reduction device 1000. In one exemplary configuration, the central waist region 1100W can define the internal channel 1110 with an internal dimension DW (shown in FIG. 2) between approximately one half millimeter and two millimeters when deployed, thereby providing a high degree of pulmonary flow restriction suitable for neonatal physiology.

Following implantation and as hemodynamic requirements of the patient 100 (shown in FIG. 1) evolve, a balloon catheter or other expansion catheter system 3000 (shown in FIGS. 5A-E) may be advanced transcatheterly into the internal channel 1110 of the transcatheter pulmonary flow reduction device 1000 under fluoroscopic or echocardiographic guidance. A balloon or other implant expansion system 3120 (shown in FIGS. 5A-E) of the expansion catheter system 3000 may be positioned such that the implant expansion system 3120 aligns with the central waist region 1100W of the transcatheter pulmonary flow reduction device 1000. Controlled inflation of the implant expansion system 3120 can result in a radial expansion of the central waist region 1100W, thereby increasing the effective internal dimension DWE (shown in FIG. 5E) of the transcatheter pulmonary flow reduction device 1000.

In certain embodiments, the internal dimension of the internal channel 1110 of the central waist region 1100W may be expanded incrementally from approximately one millimeter to larger diameters, including, but not limited to, two millimeters, three millimeters, and up to approximately five millimeters, or any dimension in between, depending on the selected balloon size and inflation pressure. This staged expansion advantageously can enable fine-tuned modulation of pulmonary blood flow without requiring surgical intervention or replacement of the transcatheter pulmonary flow reduction device 1000. Expansion may be performed during a single procedure or across multiple follow-up catheterization procedures as the patient grows.

Expansion of the internal channel 1110 of the central waist region 1100W, for example, may be facilitated by one or more plastically deformable components within the central waist region 1100W, including, but not limited to, balloon-expandable metallic structures, polymer bands, polymer or fabric septa or combinations thereof, in the manner shown and described herein with reference to the central cover region 1350 (shown in FIG. 19), the cover loop member 1352 (shown in FIG. 20), the band member 1354 (shown in FIG. 21) and/or the deformable septum member 1356 (shown in FIG. 22). In embodiments incorporating self-expanding metallic device frames 1100, the central waist region 1100W may include localized regions of reduced radial stiffness or balloon-expandable elements that permit controlled enlargement while maintaining structural integrity of the proximal and distal frame end regions 1100P, 1100D.

Advantageously, the ability to expand the internal channel 1110 of the central waist region 1100W from approximately one millimeter to approximately four millimeters can allow a single transcatheter pulmonary flow reduction device 1000 to accommodate somatic growth and changing pulmonary vascular resistance over time. This post-implant adjustability reduces the need for repeated surgical banding procedures and enables individualized, catheter-based optimization of pulmonary flow reduction throughout staged congenital heart disease management.

In selected embodiments, the cover member 1300 can extend to (or beyond) the distal strut end regions 1140D of the proximal frame end region 1100P and/or the distal frame end region 1100D such that the distal strut end regions 1140D can be fully encapsulated. The encapsulated distal strut end regions 1140D advantageously can help to reduce vessel trauma at the pulmonary artery 120 and/or inhibit endothelial ingrowth along the device frame struts 1140 or distal strut end regions 1140D of the device frame 1100. Although illustrated in FIGS. 15A-B as spanning an entire longitudinal length of the transcatheter pulmonary flow reduction device 1000 from the proximal frame end region 1100P to the distal frame end region 1100D, the cover member 1300 can extend distally from the proximal frame end region 1100P of the transcatheter pulmonary flow reduction device 1000 by any predetermined distance. The cover member 1300, in other words, can fully or partially span the longitudinal length of the transcatheter pulmonary flow reduction device 1000.

As shown in FIG. 22, the deformable septum member 1356 can be disposed within the internal channel 1110 of the central waist region 1100W. In other words, the deformable septum member 1356 can be circumferentially disposed within the internal channel 1110 and define an external size, shape, diameter, cross-section or other dimension DC that can engage an internal size, shape, diameter, cross-section or other dimension of the internal channel 1110 at the central waist region 1100W. The internal channel 1110 thereby can be partially occluded by the deformable septum member 1356 disposed at the central waist region 1100W of the device frame 1100. In selected embodiments, the deformable septum member 1356 can be affixed to, or otherwise coupled with, the central waist region 1100W. Stated somewhat differently, the circumferential deformable septum member 1356 of the cover member 1300 can be associated with, and/or cooperate with, the central waist region 1100W of the device frame 1100.

The deformable septum member 1356 can be configured to restrict or otherwise constrict the central waist region 1100W of the device frame 1100, when deployed. In selected embodiments, the deformable septum member 1356 can define a central (or internal) septum channel 1358 with an internal size, shape, diameter, cross-section or other dimension. The internal dimension of the internal septum channel 1358, for example, can be less than the internal dimension of the internal channel 1110 at the central waist region 1100W. The dimension of the internal septum channel 1358 preferably comprises the internal dimension DW as shown in FIG. 22. Thereby, the deformable septum member 1356 of the cover member 1300 can enable the internal channel 1110 of the central waist region 1100W to achieve and/or maintain the internal dimension DW when the transcatheter pulmonary flow reduction device 1000 is in the first stable expanded state. An external size, shape, diameter, cross-section or other dimension of the central waist region 1100W in the first stable expanded state, in other words, can be limited or otherwise constrained by the internal septum channel 1358 of the deformable septum member 1356.

After the initial deployment, the device frame 1100 subsequently can be re-expanded from the first stable expanded state to the second stable expanded state. The device frame 1100, for example, can be radially re-expanded from the first stable expanded state to the second stable expanded state via the expansion catheter system 3000 in the manner shown and described with reference to FIGS. 5A-E. The expansion catheter system 3000, for example, can be disposed within the internal septum channel 1358 defined by the deformable septum member 1356 and/or the internal septum channel 1110 defined by the device frame 1100. During re-expansion of the device frame 1100, the deformable septum member 1356 can plastically deform, permitting the internal dimension DW of the central waist region 1100W and/or the internal dimension of the central waist region 1100W to (gradually) increase. The internal septum channel 1358 defined by the deformable septum member 1356 thus can be radially re-expanded from the internal dimension DW to the second internal dimension DWE (shown in FIG. 5E) after deployment of the transcatheter pulmonary flow reduction device 1000. The deformable septum member 1356 advantageously can permit precisely-controlled adjustment of the external dimension of the central waist region 1100W, high-resolution adjustment of the flow adjustment and/or can maintain structural integrity of the transcatheter pulmonary flow reduction device 1000 and the cover member 1300 before, during and/or after re-expansion.

The transcatheter pulmonary flow reduction device 1000 can be delivered and deployed percutaneously. In the manner discussed above with reference to FIGS. 4A-D, for example, the transcatheter pulmonary flow reduction device 1000 can be introduced into a patient 100 (shown in FIG. 1) with congenital heart disease and deployed within a pulmonary artery 120 (shown in FIG. 1), such as a selected branch pulmonary artery 120A, 120B (shown in FIG. 28) or other lumen, of the patient 100 via any suitable catheter system or other medical device, including a delivery catheter system 2000. Additionally and/or alternatively, the deployed transcatheter pulmonary flow reduction device 1000 can be later re-expanded, recaptured, repositioned and retrieved within the body of the patient 100 and/or can be later removed from the body of the patient 100. In other words, the transcatheter pulmonary flow reduction device 1000, after being deployed within the pulmonary artery 120 of the patient 1000, can subsequently be re-expanded, recaptured, repositioned, retrieved and/or removed. The deployed transcatheter pulmonary flow reduction device 1000, in selected embodiments, can be later re-expanded, recaptured, repositioned and/or retrieved within the body of the patient 100 and/or can be later removed from the body of the patient 100 via subsequent introduction of a delivery catheter system 2000, an expansion catheter system 3000 (shown in FIGS. 5A-E) or other implant retrieval system, without limitation.

In selected embodiments, the transcatheter pulmonary flow reduction device 1000 can be delivered transvascularly via an off-the-shelf delivery catheter system 2000 having appropriate inner and outer diameter specifications compatible with the compressed transcatheter pulmonary flow reduction device 1000. Exemplary off-the-shelf delivery catheter systems 2000 can include, but are not limited to, hydrophilic-coated angiographic catheter systems such as GlideCath®-type catheters available from Terumo Medical Corporation in Somerset, New Jersey, or similar commercially-available devices. The transcatheter pulmonary flow reduction device 1000, for example, can be backloaded into the proximal end of the delivery catheter system 2000 and advanced through the vasculature to the deployment site. Use of the off-the-shelf delivery catheter systems 2000 advantageously can leverage widely-available catheter platforms, enable rapid integration into existing interventional workflows and/or reduce a need for specialized delivery hardware.

The transcatheter pulmonary flow reduction device 1000 alternatively can be delivered transvascularly via a customized or otherwise specialized delivery catheter system 2000. The transcatheter pulmonary flow reduction device 1000, for example, can be delivered using a custom delivery catheter system 2000 that can be provided as part of an integrated delivery system. In selected embodiments, the delivery catheter system 2000 can be constructed as a composite structure, incorporating coiled and/or braided metallic wire embedded within or laminated between one or more polymer layers. A pitch of a coil, a braid density and/or a polymer wall thickness can vary along a length of the delivery catheter system 2000 for achieving region-specific mechanical behavior. In selected embodiments, a distal segment of the delivery catheter system 2000 can be highly flexible to safely navigate tortuous neonatal anatomy; while, a proximal segment of the delivery catheter system 2000 can exhibit increased column strength to allow effective pushability and resistance to compressive buckling. The custom delivery catheter system 2000 advantageously can provide optimized balance of trackability, flexibility and control during implantation.

An off-the-shelf short vascular sheath (not shown), for example, can be introduced at an access site on the body of the patient 100 (shown in FIG. 1). A commercially-available delivery catheter system 2000, such as a GlideCath®—type hydrophilic catheter available from Terumo Medical Corporation in Somerset, New Jersey, with a suitable inner diameter can be advanced through the vascular sheath and tracked to an intended deployment location within the pulmonary artery 120 (shown in FIG. 1) of the patient 100 over a guide wire 2400 (shown in FIG. 26). After proper positioning is confirmed and the guide wire 2400 is removed, the transcatheter pulmonary flow reduction device 1000 can be backloaded into a proximal end region of the delivery catheter system 2000 and advanced axially through a lumen (not shown) of the delivery catheter system 2000 to the deployment location. Use of the commercially-available delivery catheter system 2000 advantageously can leverage standard interventional tools and/or provide a straightforward, modular approach for introducing and deploying the transcatheter pulmonary flow reduction device 1000.

In selected embodiments, the transcatheter pulmonary flow reduction device 1000 can be preloaded or otherwise provided within a distal tip or catheter distal end region 2100 (shown in FIGS. 4A-D) of a custom delivery catheter system 2000. The transcatheter pulmonary flow reduction device 1000, in other words, can be provided as a part of a preloaded catheter assembly. The transcatheter pulmonary flow reduction device 1000 thereby can be advanced through an off-the-shelf or custom long sheath (not shown) to the intended deployment location within the pulmonary artery 120 of the patient 100 over a guide wire 2400 (shown in FIG. 26). The guide wire 2400 then can be removed, and the preloaded catheter assembly can be tracked along the sheath to the deployment location. The sheath advantageously can provide proximal support, facilitate safe navigation through tortuous anatomy and/or protect the transcatheter pulmonary flow reduction device 1000 during transit to the intended deployment location. After reaching the deployment location, the deployment location can be advanced through the sheath to enable precise deployment of the transcatheter pulmonary flow reduction device 1000.

In another embodiment, the transcatheter pulmonary flow reduction device 1000 can be preloaded within the tip or catheter distal end region 2100 of a custom delivery catheter system 2000. The transcatheter pulmonary flow reduction device 1000, in other words, can be provided as a part of a preloaded catheter assembly. Here, the delivery catheter system 2000 can include a flexible, curved distal segment (not shown) that can be configured for navigating a vessel pathway of the patent 100 without requiring a guide wire 2400. The preloaded catheter assembly thereby can be advanced through a short sheath (not shown) to the intended deployment location within the pulmonary artery 120 of the patient 100. The inherent curvature and flexibility of the distal segment of the delivery catheter system 2000 advantageously can enable atraumatic tracking of the preloaded catheter assembly through neonatal pulmonary branches of the patient 100 while eliminating a need for wire exchange or wire navigation.

Additionally and/or alternatively, the transcatheter pulmonary flow reduction device 1000 can be preloaded within a custom delivery catheter system 2000 that is configured for guide wire-based navigation. A short vascular sheath can be introduced into the body of the patient 100, and a guide wire 2400 can be positioned or otherwise disposed in an intended deployment location. The preloaded transcatheter pulmonary flow reduction device 1000 thereby can be advanced over the guide wire 2400 through the sheath and tracked to the intended deployment location. This delivery catheter system 2000 advantageously can provide enhanced control and trackability of the transcatheter pulmonary flow reduction device 1000 via the guide wire 2400, particularly in cases requiring precise navigation along tortuous or angled vessel geometries within the patient 100.

The tracking and sheath configurations described herein can enable the transcatheter pulmonary flow reduction device 1000 to be advanced safely and accurately to the intended deployment location within the pulmonary artery 120 of the patient 100. The transcatheter pulmonary flow reduction device 1000 thus can temporarily be secured to, or otherwise engage, the delivery catheter system 2000 during transit to the intended deployment location. The delivery catheter system 2000 can include an optional attachment and decoupling mechanism (or system) (not shown) for allowing controlled and/or deliberate release of the transcatheter pulmonary flow reduction device 1000 at the intended deployment location.

The attachment and decoupling mechanism can be provided in any suitable manner. In selected embodiments, the attachment and decoupling mechanism can be provided as a tethered suture loop (not shown). The suture loop can comprise a suture that has a predetermined length of suture material with proximal and distal end regions and that forms a suture loop. The suture loop can be disposed around the proximal frame end region 1100P (shown in FIG. 2) of the transcatheter pulmonary flow reduction device 1000. The proximal and distal end regions of the suture can be routed proximally through the delivery catheter system 2000 and terminate at a delivery system handle (not shown) of the delivery catheter system 2000.

The transcatheter pulmonary flow reduction device 1000 can remained secured to the delivery catheter system 2000 while the suture loop is intact. When the transcatheter pulmonary flow reduction device 1000 is positioned within the intended deployment location, the suture loop can disengage from the transcatheter pulmonary flow reduction device 1000. One of the end regions of the suture loop, for example, can be pulled for disengaging the transcatheter pulmonary flow reduction device 1000. The transcatheter pulmonary flow reduction device 1000 thereby can be released from the delivery catheter system 2000. The suture loop advantageously can provide a simple, low-profile attachment and decoupling mechanism that is compatible with small-diameter access requirements associated, for example, with the small vasculature of neonatal, infant, toddlers, young children and other pediatric patients.

Additionally and/or alternatively, the tethered suture loop can be disposed around the proximal frame end region 1100P (shown in FIG. 2) of the transcatheter pulmonary flow reduction device 1000, but only one of the end regions of the suture is configured for extending to the delivery system handle of the delivery catheter system 2000. The other end region of the suture can terminate or otherwise be disposed within a catheter lumen (not shown) of the delivery catheter system 2000. The transcatheter pulmonary flow reduction device 1000 thereby can be released from the delivery catheter system 2000 via application of tension to the externalized suture. The tension can allow the suture to break, slip free, be cut or otherwise disengage the transcatheter pulmonary flow reduction device 1000 and to subsequently be withdrawn through the catheter lumen of the delivery catheter system 2000. Once the tethered suture loop disengages the transcatheter pulmonary flow reduction device 1000, the transcatheter pulmonary flow reduction device 1000 can be decoupled from the delivery catheter system 2000. Use of the tethered suture loop advantageously can simplify proximal routing and/or can help to reduce frictional drag within very small delivery catheter systems 2000.

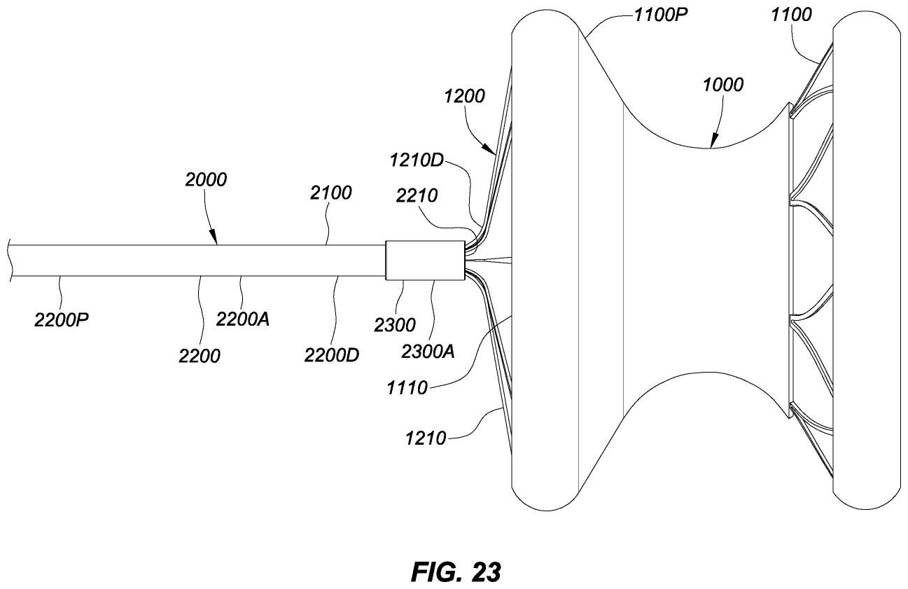

The attachment and decoupling mechanism, in selected embodiments, can be provided via a micro-treaded interface system. An exemplary embodiment of the delivery catheter system 2000 with a micro-treaded interface system is illustrated in FIG. 23. Turning to FIG. 23, the delivery catheter system 2000 is shown as including a catheter and sheath assembly with an elongated delivery shaft member (or system) 2200 with proximal and distal shaft end regions 2200P, 2200D. The proximal shaft end region 2200P, for example, can be configured for coupling with a distal end region of a flexible wire, a guide wire 2400 (shown in FIG. 26), a hypotube and/or a laser-cut hypotube (not shown) of the delivery catheter system 2000. The delivery shaft member 2200 can be provided in any suitable manner. In selected embodiments, the delivery shaft member 2200 can be provided as a deployment rod system, a threaded deployment rod system, a micro-threaded deployment rod system 2200A, a hypotube system or a braided microcatheter system, without limitation. The delivery shaft member 2200 optionally can be provided with variable cut geometries and/or pitches to tune a flexibility of the delivery shaft member 2200 along a longitudinal length of the delivery shaft member 2200. The delivery shaft member 2200 can be formed or otherwise manufactured from any suitable material. Exemplary delivery shaft materials can include stainless steel, without limitation.

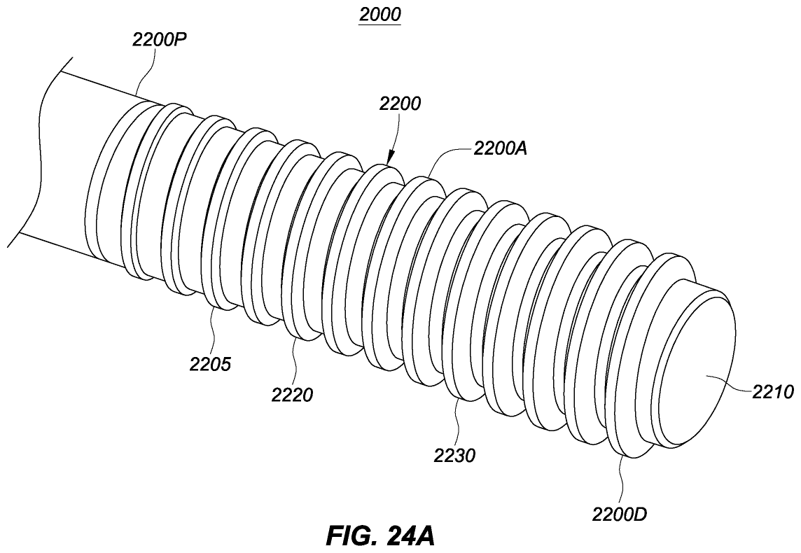

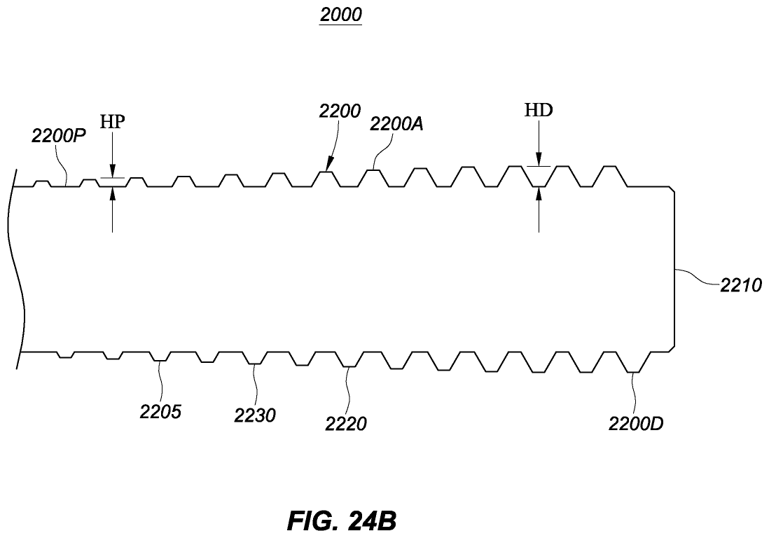

An exemplary embodiment of the micro-threaded deployment rod system 2200A is shown in FIGS. 24A-B. Turning to FIGS. 24A-B, the micro-threaded deployment rod system 2200A is shown as comprising an elongated annular rod body 2205 with the proximal and distal shaft end regions 2200P, 2200D. The annular rod body 2205 can define an axial central (or internal) rod channel 2210 and/or an external rod periphery 2220. The axial internal rod channel 2210 can extend from the proximal shaft end region 2200P to the distal shaft end region 2200D.

As shown in FIGS. 24A-B, the external rod periphery 2220 can include or otherwise define one or more external rod threads 2230. The annular rod body 2205, in other words, can comprise an externally-threaded rod periphery 2220. In selected embodiments, the external rod threads 2230 of the annular rod body 2205 can comprise micro-threads. The external rod threads 2230 defined on the external rod periphery 2220 can have a predetermined height. The predetermined thread height of the external rod threads 2230 can comprise any predetermined thread height, which can be uniform and/or different along a longitudinal length of the annular rod body 2205. The thread height of external rod threads 2230, in other words, can be straight and/or tapered. For example, a predetermined thread height HD of the external rod threads 2230 at the distal shaft end region 2200D of the annular rod body 2205 can be greater than a predetermined thread height HP of the external rod threads 2230 at the proximal shaft end region 2200P of the annular rod body 2205 as illustrated in FIGS. 24A-B.

Returning to FIG. 23, an implant interface member 2300 can be disposed at the distal shaft end region 2200D of the delivery shaft member 2200. If the delivery shaft member 2200 comprises a micro-threaded deployment rod system 2200A, for example, the implant interface member 2300 can be provided as a micro-threaded collar system 2300A for cooperating with the micro-threaded deployment rod system 2200A. The implant interface member 2300 can be formed or otherwise manufactured from any suitable material. Exemplary implant interface materials can include stainless steel and/or Nitinol, without limitation.

The implant interface member 2300 advantageously can be adapted for engaging the transcatheter pulmonary flow reduction device 1000. The transcatheter pulmonary flow reduction device 1000, in other words, can be temporarily secured to the delivery shaft member 2200 of the delivery catheter system 2000 via the implant interface member 2300. The transcatheter pulmonary flow reduction device 1000, in other words, can be tethered or otherwise affixed to the distal shaft end region 2200D of the delivery shaft member 2200 via the implant interface member 2300. Although shown and described with reference to FIG. 23 as engaging a device retrieval system 1200 of the transcatheter pulmonary flow reduction device 1000 for purposes of illustration only, the implant interface member 2300 can engage the transcatheter pulmonary flow reduction device 1000 in any suitable manner.

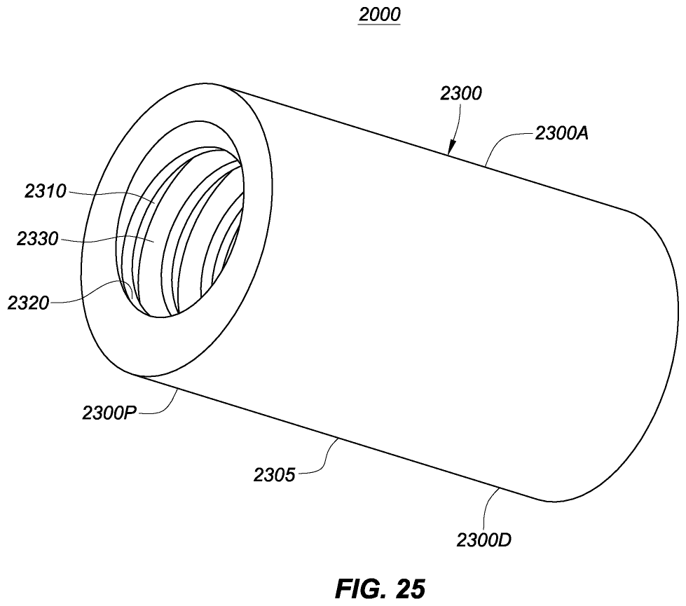

An exemplary embodiment of the micro-threaded collar system 2300A is shown in FIG. 25. Turning to FIG. 25, the micro-threaded collar system 2300A is illustrated as comprising an elongated annular collar body 2305 with the proximal and distal collar end regions 2300P, 2300D. In selected embodiments, the annular collar body 2305 can comprise a laser-cut hypotube. The annular collar body 2305 can define an axial central (or internal) collar channel 2310 being bounded by an internal channel periphery 2320. The axial internal collar channel 2310 can extend from the proximal collar end region 2300P to the distal collar end region 2300D. As shown in FIG. 25, the internal channel periphery 2320 can include or otherwise define one or more internal channel threads 2330. The annular collar body 2305, in other words, can comprise an internally-threaded channel periphery 2320. In selected embodiments, the internal channel threads 2330 of the annular collar body 2305 can comprise micro-threads.

The internal channel threads 2330 defined on the internal channel periphery 2320 can extend into the internal channel periphery 2320 and can have a predetermined height. The predetermined thread height of the internal channel threads 2330 can comprise any predetermined thread height, which can be uniform and/or different along a longitudinal length of the annular collar body 2305. The thread height of internal channel threads 2330, in other words, can be straight and/or tapered. The internal channel threads 2330 defined by the micro-threaded collar system 2300A preferably are configured to engage or otherwise cooperate with the external rod threads 2230 of the micro-threaded deployment rod system 2200A. In other words, the micro-threaded deployment rod system 2200A and the micro-threaded collar system 2300A can be coupled, in selected embodiments, via an engagement, interference fit or other cooperation between the internal channel threads 2330 defined by the micro-threaded collar system 2300A and the external rod threads 2230 of the micro-threaded deployment rod system 2200A.

Returning again to FIG. 23, the delivery shaft member 2200, when coupled with the transcatheter pulmonary flow reduction device 1000 via the implant interface member 2300, can axially advance or retract the transcatheter pulmonary flow reduction device 1000 in a controlled manner. A guide wire 2400 (shown in FIG. 26), for example, can be disposed within an internal axial channel 2210 defined by the delivery shaft member 2200 and advantageously can be used to track a position of the transcatheter pulmonary flow reduction device 1000 during introduction and deployment within the pulmonary artery 120. In selected embodiments, the delivery catheter system 2000 can lock the delivery shaft member 2200 in place during delivery and/or deployment of the transcatheter pulmonary flow reduction device 1000, preventing premature detachment of the transcatheter pulmonary flow reduction device 1000 from the delivery catheter system 2000.

If comprising the micro-threaded deployment rod system 2200A and/or the micro-threaded collar system 2300A, for example, the delivery catheter system 2000 can utilize the cooperation between the internal channel threads 2330 defined by the micro-threaded collar system 2300A and the external rod threads 2230 of the micro-threaded deployment rod system 2200A to prevent any premature detachment of the transcatheter pulmonary flow reduction device 1000. The delivery catheter system 2000, in other words, can exploit the cooperation between the internal channel threads 2330 defined by the micro-threaded collar system 2300A and the external rod threads 2230 of the micro-threaded deployment rod system 2200A to axially advance or retract the transcatheter pulmonary flow reduction device 1000.

During deployment of the transcatheter pulmonary flow reduction device 1000, a rotational torque can be applied to the guide wire 2400 (shown in FIG. 26) or hypotube, causing the micro-threaded deployment rod system 2200A to be at least partially unthreaded from the micro-threaded collar system 2300A and thereby release the transcatheter pulmonary flow reduction device 1000. The micro-threaded deployment rod system 2200A, in other words, can be partially or completely unthreaded from the micro-threaded collar system 2300A for releasing the transcatheter pulmonary flow reduction device 1000 from the delivery catheter system 2000. The delivery catheter system 2000 advantageously can provide a mechanically-robust attachment mechanism with predictable, controlled detachment of the transcatheter pulmonary flow reduction device 1000, while allowing secure retention of the transcatheter pulmonary flow reduction device 1000 during tracking through tortuous vasculature of the patient 100.

An optional handle assembly (not shown) can be disposed at the proximal shaft end region 2200P of the delivery shaft member 2200, and/or am implant expansion system, such as the implant expansion system 3120 (shown in FIGS. 5A-E), optionally can be disposed at the distal shaft end region 2200P of the delivery shaft member 2200. The handle assembly advantageously can support one-handed operation of the delivery catheter system 2000 and controlled sheath retraction for gradual release and deployment of the transcatheter pulmonary flow reduction device 1000 within the pulmonary artery 120. In selected embodiments, the delivery system 2000 can support implantation, deployment, recapture, repositioning, retrieval and/or removal of the transcatheter pulmonary flow reduction device 1000, for example, under fluoroscopic and/or echocardiographic guidance. The delivery system 2000 optionally can be configured for introducing and deploying the transcatheter pulmonary flow reduction device 1000 within the small vasculature of neonatal, infant, toddlers, young children and other pediatric patients.

In selected embodiments, the delivery shaft member 2200 can be configured to engage the device retrieval system 1200 of the transcatheter pulmonary flow reduction device 1000. The delivery shaft member 2200 optionally can be configured to removably engage the device retrieval system 1200. The implant interface member 2300, for example, can engage the coupling device (or member) 1240 of the transcatheter pulmonary flow reduction device 1000 in the manner illustrated in FIG. 26. Additionally and/or alternatively, the implant interface member 2300 can engage the coupling device (or member) 1260 (shown in FIGS. 11A-B) and/or the coupling device (or member) 1270 (shown in FIGS. 13A-B) of the transcatheter pulmonary flow reduction device 1000.

As shown in FIG. 26, for example, the implant interface member 2300 can include an optional interface engagement device (or member) 2340 for engaging the coupling device 1240, 1260, 1270, 1280 of the device retrieval system 1200. The coupling device 1240, 1260, 1270, 1280, in selected embodiments, can define at least one engagement opening 1244, 1264, 1274, 1284 in the manner discussed in more detail above with reference to FIGS. 9A-B, 11A-B and 13A-B. The interface engagement device 2340 can be configured to be received within, or otherwise engage, the engagement opening 1244, 1264, 1274, 1284 of the relevant coupling device 1240, 1260, 1270, 1280. Thereby, the transcatheter pulmonary flow reduction device 1000 can be tethered, affixed or otherwise disposed on the catheter distal end region 2100 of a delivery catheter system 2000.

The interface engagement device 2340 can be provided in any suitable manner. For instance, the interface engagement device 2340 can be provided as a coupler paddle (or paddle-based) system (or member or means) 2350 as illustrated in FIGS. 27A-B. The coupler paddle system 2350 can include a raised member 2354 that is coupled with the distal collar end region 2300D of the annular collar body 2305 via a bendable or otherwise flexible coupler member 2352. The raised member 2354 has a predetermined size, shape, orientation, diameter, cross-section or other dimension that is suitable for being received within, or otherwise engaging, the engagement opening 1244, 1264, 1274, 1284 of the relevant coupling device 1240, 1260, 1270, 1280.

In selected embodiments, the raised member 2354 can comprise one or more wedges, one or more shims, one or more embossments or one or more other protrusions, without limitation, that can be configured to increase an engagement force and/or a release force with the engagement opening 1244, 1264, 1274, 1284 of the relevant coupling device 1240, 1260, 1270, 1280 of the transcatheter pulmonary flow reduction device 1000. The dimension of the raised member 2354 advantageously can help to push the raised member 2354 further into the mating engagement opening 1244, 1264, 1274, 1284, enhancing a locking interface and/or further securing the transcatheter pulmonary flow reduction device 1000 to the delivery catheter system 2000 during tracking and positioning of the transcatheter pulmonary flow reduction device 1000 during implantation and/or deployment. The dimension of the raised member 2354, for example, can be varied to tune the engagement force and/or the release force and to help ensure controlled detachment while maintaining reliable retention during navigation through tortuous vasculature. The coupler paddle system 2350 can be formed or otherwise manufactured from any suitable material. Exemplary suitable materials can include stainless steel and/or Nitinol, without limitation. In selected embodiments, the coupler paddle system 2350 can be provided as a Nitinol shapeset coupler paddle system.

Turning to FIG. 27A, for example, the coupler paddle system 2350 is shown as being coupled with, or otherwise integrated with, the distal collar end region 2300D of the annular collar body 2305. The coupler paddle system 2350, in other words, can extend distally from the distal collar end region 2300D of the implant interface member 2300. In selected embodiments, the coupler paddle system 2350 can have an initial coupler state in which the coupler paddle system 2350 can be biased toward a longitudinal axis of the internal collar channel 2310 defined by the implant interface member 2300. The coupler paddle system 2350, in other words, can form a predetermined angle Θ with the longitudinal axis of the internal collar channel 2310. The predetermined angle Θ can comprise any suitable predetermined angle, such as an angle between zero degrees and thirty degrees, without limitation.

In operation, the coupler paddle system 2350 can be integrated into the delivery catheter system 2000 and can extend distally from the distal collar end region 2300D of the annular collar body 2305 as illustrated in FIGS. 27A-B. Prior to deployment of the transcatheter pulmonary flow reduction device 1000, the coupler paddle system 2350 can be disposed in the initial coupler state. The guide wire 2400 can advance distally through the internal collar channel 2310 defined by the annular collar body 2305 and engage the coupler paddle system 2350. The engagement between the guide wire 2400 and the coupler paddle system 2350 can bend the coupler paddle system 2350 from the initial coupler state into an expanded coupler state as shown in FIG. 27B. The advancing guide wire 2400, in other words, can bend the flexible coupler member 2352 such that the coupler paddle system 2350 is axially aligned with the internal collar channel 2310 defined by the implant interface member 2300.

When the coupler paddle system 2350 is in the expanded coupler state, the raised member 2354 can be disposed within or otherwise engage the engagement opening 1244, 1264, 1274, 1284 of the relevant coupling device 1240, 1260, 1270, 1280. Stated somewhat differently, the guide wire 2400 can be pre-threaded through the annular collar body 2305, forcing the inwardly-biased flexible coupler member 2352 to expand outwardly such that the raised member 2354 can be disposed within or otherwise engage the engagement opening 1244, 1264, 1274, 1284 of the coupling device 1240, 1260, 1270, 1280. The guide wire 2400 can maintain the engagement between the raised member 2354 and the engagement opening 1244, 1264, 1274, 1284. The transcatheter pulmonary flow reduction device 1000 thereby can be coupled or otherwise secured with the catheter distal end region 2100 of the delivery catheter system 2000. The catheter distal end region 2100 of the delivery catheter system 2000 then can be advanced through the vasculature (not shown) of the patent 100 and into the heart 110 until the device frame 1100 is positioned at a predetermined location within the pulmonary artery 120 in the manner discussed in more detail with reference to FIGS. 4A-D.

Once positioned at the predetermined location within the pulmonary artery 120, the device frame 1100 can be implanted and otherwise deployed in the patient 100. To decouple the transcatheter pulmonary flow reduction device 1000 from the catheter distal end region 2100 of the delivery catheter system 2000, the guide wire 2400 can be removed or retracted proximally within the internal collar channel 2310 defined by the annular collar body 2305. The guide wire 2400 thereby can disengage the coupler paddle system 2350, permitting the coupler paddle system 2350 to return to the initial coupler state. The retracted guide wire 2400, in other words, can permit the flexible coupler member 2352 to revert to the initial state and again form the predetermined angle Θ with the longitudinal axis of the internal collar channel 2310. Stated somewhat differently, removal of the guide wire 2400 can permit the flexible coupler member 2352 to elastically return inwardly. Thereby, the raised member 2354 can retract from the engagement opening 1244, 1264, 1274, 1284 of the relevant coupling device 1240, 1260, 1270, 1280, decoupling the transcatheter pulmonary flow reduction device 1000 from the catheter distal end region 2100 of the delivery catheter system 2000. The coupler paddle system 2350 advantageously can provide a secure, reversible attachment system that offers high tactile feedback during implantation and/or deployment of the transcatheter pulmonary flow reduction device 1000.

Although shown and described with reference to FIGS. 26 and 27A-B as comprising a delivery catheter system for implanting and deploying the transcatheter pulmonary flow reduction device 1000, the delivery catheter system 2000 additionally and/or alternatively can be configured for recapture, repositioning, retrieval and/or removal of the transcatheter pulmonary flow reduction device 1000 after deployment. The delivery catheter system 2000, in other words, can be provided as the expansion catheter system 3000 (shown in FIGS. 5A-E) or other implant retrieval system that can be utilized to engage the device retrieval system 1200 and to recapture, reposition, retrieve and/or remove the transcatheter pulmonary flow reduction device 1000 within the pulmonary artery 120 (shown in FIG. 1).

In some embodiments, a transcatheter pulmonary flow reduction device 1000 may be deployed within a branch pulmonary artery 120A, 120B of a patient 100 (shown in FIG. 1) to partially restrict blood flow while maintaining perfusion to downstream branching vessels as illustrated in FIG. 28. Uncovered (or partially covered) regions of the transcatheter pulmonary flow reduction device 1000, including portions of the transcatheter pulmonary flow reduction device 1000 that are adjacent to one or more saddle regions 1150 (shown in FIG. 28) of the transcatheter pulmonary flow reduction device 1000, may permit blood flow through open frame cells 1144, 1148 of the device frame 1100, allowing perfusion to side branches or bifurcating vessels downstream of the transcatheter pulmonary flow reduction device 1000. The saddle regions 1150 can be located proximally and/or distally to the central waist region 1100W in the manner illustrated in FIG. 28. This configuration may enable controlled flow reduction across the transcatheter pulmonary flow reduction device 1000 while preserving physiologic flow patterns to adjacent vasculature.

As shown in FIG. 28, the transcatheter pulmonary flow reduction device 1000 can be deployed in a branch pulmonary artery 120A, 120B of the patient 100 (shown in FIG. 1). An annular cover member 1300 optionally can be disposed circumferentially around the external periphery 1120 of the transcatheter pulmonary flow reduction device 1000 such as in the manners discussed in more detail above with reference to the annular cover member 1300 of FIGS. 15A-B, 16A-B, 17A-B, 18A-B, 19, 20, 21 and/or 22, without limitation. Saddle regions 1150 can be located proximally and/or distally to the central waist region 1100W and/or may create one or more unrestricted flow regions (not shown) of relatively unrestricted blood movement within and around the transcatheter pulmonary flow reduction device 1000. The unrestricted flow regions advantageously can promote washout and/or reduce blood residence time adjacent to the external periphery 1120 (shown in FIG. 2) of the transcatheter pulmonary flow reduction device 1000, helping to mitigate thrombus formation and/or reduce the risk of occlusion. The combination of a flow-restrictive central waist region 1100W and open saddle regions 1150 may provide a balance between effective flow reduction and maintenance of favorable hemodynamics within the branch pulmonary artery 120A, 120B. Restriction of blood flow 122 through the transcatheter pulmonary flow reduction device 1000, flow through at least one uncovered portion of the transcatheter pulmonary flow reduction device 1000 to the branch pulmonary arteries 120A, 120B and/or free movement of blood within the saddle regions 1150 above and below the central waist region 1100W advantageously can help to prevent thrombosis.

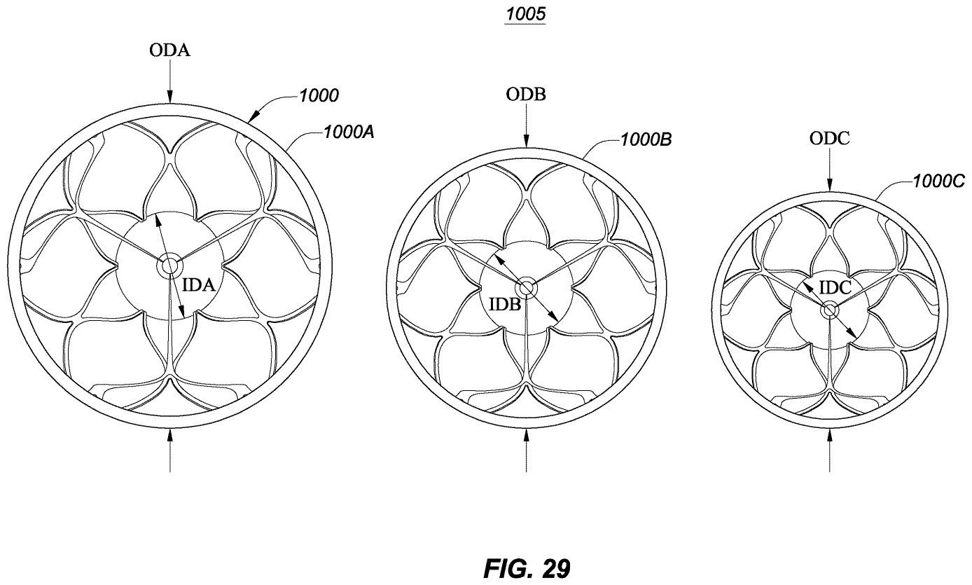

In some embodiments, the transcatheter pulmonary flow reduction device 1000 may be provided as a collection 1005 of transcatheter pulmonary flow reduction devices 1000 as illustrated in FIG. 29. The transcatheter pulmonary flow reduction devices 1000 in the collection 1005 can have a plurality of different sizes and/or different configurations for accommodating anatomical variability of pulmonary arteries across neonatal, infant, and pediatric patients 100 (shown in FIG. 1). The illustrated embodiments may differ in one or more dimensional parameters, including, but not limited to, expanded outer diameter, waist diameter, axial length, saddle diameter, or taper angle, while maintaining a common functional architecture for flow reduction.

As shown in FIG. 29, for example, a first transcatheter pulmonary flow reduction device 1000A can have a first inner diameter IDA and a first second outer diameter ODA. A second transcatheter pulmonary flow reduction device 1000B can have a second inner diameter IDB and a second outer diameter ODB; whereas, a third transcatheter pulmonary flow reduction device 1000C can have a third inner diameter IDC and a third second outer diameter ODC. The first inner diameter IDA of the first transcatheter pulmonary flow reduction device 1000A can be greater than the second inner diameter IDB of the second transcatheter pulmonary flow reduction device 1000B, which, in turn, can be greater than the third inner diameter IDC of the third transcatheter pulmonary flow reduction device 1000C. Additionally and/or alternatively, the first outer diameter ODA of the first transcatheter pulmonary flow reduction device 1000A can be greater than the second outer diameter ODB of the second transcatheter pulmonary flow reduction device 1000B, which, in turn, can be greater than the third outer diameter ODC of the third transcatheter pulmonary flow reduction device 1000C.

Each of the transcatheter pulmonary flow reduction device 1000 in the collection 1005 may be configured for delivery via a low-profile delivery system 2000 (shown in FIGS. 4A-D), such as a catheter having an inner diameter of approximately one millimeter or greater, with the transcatheter pulmonary flow reduction device 1000 being radially compressed for delivery and expansion upon deployment. Despite differences in expanded dimensions, the transcatheter pulmonary flow reduction devices 1000 may share similar frame patterns, strut geometries, and material compositions, enabling predictable mechanical behavior and consistent flow-reduction performance across the relevant size ranges.

As used herein, a phrase in the form of at least one of A, B, C and D herein is to be construed as meaning one or more of A, one or more of B, one or more of C and/or one or more of D. Likewise, a phrase in the form of A, B, C or D as used herein is to be construed as meaning A or B or C or D. For example, a phrase in the form of A, B, C or a combination thereof is to be construed as meaning A or B or C or any combination of A, B and/or C.

The described embodiments are susceptible to various modifications and alternative forms, and specific examples thereof have been shown by way of example in the drawings and are herein described in detail. It should be understood, however, that the described embodiments are not to be limited to the particular forms or methods disclosed, but to the contrary, the present disclosure is to cover all modifications, equivalents, and alternatives.

What is claimed is:

1. A transcatheter pulmonary flow reduction device for adjusting blood flow through a pulmonary artery of a patient, comprising:
a flexible device frame having an annular waist region in axial alignment between an annular proximal frame end region and an annular distal frame end region and being in an implantation state for facilitating insertion into the pulmonary artery of the patient, the annular proximal frame end region, the annular waist region and the annular distal frame end region each defining an external periphery and cooperating to define an internal channel extending from the annular proximal frame end region to the annular distal frame end region,
wherein said device frame is configured to be deployed within the pulmonary artery and radially expanded from the implantation state to a first stable expanded state with the external periphery of the annular proximal frame end region and the external periphery of the annular distal frame end region each being adapted to engage the pulmonary artery and with the internal channel of the annular waist region having an internal cross-section being adapted to radially expand to a predetermined first cross-section that is less than a cross-section of the pulmonary artery for restricting the blood flow through the pulmonary artery via said device frame, and
wherein said deployed device frame is configured to be subsequently radially re-expanded from the first stable expanded state to a second stable expanded state with the internal cross-section of the annular waist region being further expanded to a predetermined second cross-section that is less than the cross-section of the pulmonary artery and that is greater than the predetermined first cross-section for adjusting the restricted blood flow through the pulmonary artery via said device frame.

2. The transcatheter pulmonary flow reduction device of claim 1, wherein the transcatheter pulmonary flow reduction device is configured for adjusting blood flow in a pediatric patient.

3. The transcatheter pulmonary flow reduction device of claim 1, wherein the transcatheter pulmonary flow reduction device is configured for adjusting blood flow in an adult patient.

4. The transcatheter pulmonary flow reduction device of claim 1, wherein the external periphery of the annular proximal frame end region and the external periphery of the annular distal frame end region each is adapted to engage an internal surface of the pulmonary artery.

5. The transcatheter pulmonary flow reduction device of claim 4, wherein the external periphery of the annular proximal frame end region and the external periphery of the annular distal frame end region is adapted to maintain the engagement with the pulmonary artery when said device frame is in the second stable expanded state.

6. The transcatheter pulmonary flow reduction device of claim 1, wherein the external peripheries of the annular proximal frame end region, the annular waist region and the annular distal frame end region define an hourglass shape in the first stable expanded state.

7. The transcatheter pulmonary flow reduction device of claim 6, wherein the external peripheries of the annular proximal frame end region, the annular waist region and the annular distal frame end region define maintain the hourglass shape in the second stable expanded state.

8. The transcatheter pulmonary flow reduction device of claim 1, wherein said device frame is configured for implantation within the pulmonary artery of the patient via a surgical procedure.

9. The transcatheter pulmonary flow reduction device of claim 1, wherein said device frame is configured for implantation and deployment within the pulmonary artery of the patient via a delivery catheter system.

10. The transcatheter pulmonary flow reduction device of claim 1, wherein said device frame comprises a self-expanding device frame.

11. The transcatheter pulmonary flow reduction device of claim 10, wherein said device frame is formed from a shape-memory alloy.

12. The transcatheter pulmonary flow reduction device of claim 1, wherein said device frame is formed from stainless steel or a cobalt-chromium alloy.

13. The transcatheter pulmonary flow reduction device of claim 1, wherein said device frame is configured for re-expansion from the first stable expanded state to the second stable expanded state via an expansion catheter system.

14. The transcatheter pulmonary flow reduction device of claim 1, wherein said device frame is configured for re-expansion from the first stable expanded state to the second stable expanded state via a second medical procedure that is subsequent to a first medical procedure during which said device frame is expanded from the implantation state to the first stable expanded state.

15. The transcatheter pulmonary flow reduction device of claim 1,
wherein the annular proximal frame end region of said device frame comprises a first annular arrangement of device frame struts, and
wherein the annular distal frame end region of said device frame comprises a second annular arrangement of device frame struts.

16. The transcatheter pulmonary flow reduction device of claim 15, wherein the first and second annular arrangements of device frame struts include at least one elongated frame strut that extends from the annular proximal frame end region to the annular distal frame end region of said device frame.

17. The transcatheter pulmonary flow reduction device of claim 15, wherein the first and second annular arrangements of device frame struts comprise a plurality of elongated frame struts that extend from the annular proximal frame end region to the annular distal frame end region of said device frame.

18. The transcatheter pulmonary flow reduction device of claim 15, wherein the first and second annular arrangements of device frame struts comprise a plurality of meandering device frame struts that defines one or more circumferential rows of frame cells disposed around the external periphery of said device frame.

19. The transcatheter pulmonary flow reduction device of claim 18, wherein the frame cells are defined between respective pairs of adjacent device frame struts.

20. The transcatheter pulmonary flow reduction device of claim 18, wherein the circumferential rows of frame cells include at least one circumferential row of growth frame cells.

21. The transcatheter pulmonary flow reduction device of claim 20, wherein the at least one circumferential row of growth frame cells is associated with the annular waist region of said device frame.

22. The transcatheter pulmonary flow reduction device of claim 21, wherein the growth frame cells associated with the annular waist region of said device frame each have a first dimension when said device frame is in the first stable expanded state and a second dimension that is greater than the first dimension when said device frame is in the second stable expanded state.

23. The transcatheter pulmonary flow reduction device of claim 1, wherein said device frame defines a predetermined pattern of frame cells.

24. The transcatheter pulmonary flow reduction device of claim 23, wherein the predetermined pattern of frame cells includes a plurality of circumferential rows of frame cells disposed around the external periphery of said device frame, each of the circumferential rows having a predetermined number of frame cells.

25. The transcatheter pulmonary flow reduction device of claim 24, wherein at least one of the circumferential rows of frame cells comprises a predetermined number of re-expandable growth frame cells.

26. The transcatheter pulmonary flow reduction device of claim 24, wherein the plurality of circumferential rows of frame cells includes at least one proximal circumferential row of frame cells being associated with the proximal frame end region of said device frame, at least one distal circumferential row of frame cells being associated with the distal frame end region of said device frame and at least one central circumferential row of frame cells being disposed between the at least one proximal circumferential row of frame cells and the at least one distal circumferential row of frame cells and being associated with the annular waist region of said device frame.

27. The transcatheter pulmonary flow reduction device of claim 26, wherein the at least one central circumferential row of frame cells comprises a predetermined number of re-expandable growth frame cells.

28. The transcatheter pulmonary flow reduction device of claim 23, wherein the predetermined pattern of frame cells includes three circumferential rows of frame cells disposed around the external periphery of said device frame, each of the circumferential rows having six frame cells.

29. The transcatheter pulmonary flow reduction device of claim 23, wherein the predetermined pattern of frame cells includes three circumferential rows of frame cells disposed around the external periphery of said device frame, each of the circumferential rows having eight frame cells.

30. The transcatheter pulmonary flow reduction device of claim 23, wherein the predetermined pattern of frame cells includes three circumferential rows of frame cells disposed around the external periphery of said device frame, each of the circumferential rows having ten frame cells.
